(19) United States
(12) Patent Application Publication
(10) Pub. No.: US 2019/0256830 A1
(43) Pub. Date: Aug. 22, 2019

(54) METHOD FOR CELL-SPECIFICALLY CONTROLLING NUCLEASE

(71) Applicant: KYOTO UNIVERSITY, Kyoto (JP)

(72) Inventors: Hirohide Saito, Kyoto (JP); Moe Hirosawa, Kyoto (JP)

(73) Assignee: KYOTO UNIVERSITY, Kyoto (JP)

(21) Appl. No.: 16/313,328

(22) PCT Filed: May 18, 2017
(86) PCT No.: PCT/JP2017/018742

§ 371 Date: Apr. 10, 2019

(30) Foreign Application Priority Data
Jun. 27, 2016 (JP) 2016-126944

(51) Int. Cl. C12N 9/22; C12N 15/11

(52) U.S. Cl. CPC C12N 9/22; C12N 2310/20; C12N 15/11

(57) ABSTRACT

A method is provided for regulating the activity of a nuclease in response to a cell-specific miRNA by using a nucleic acid sequence specifically recognized by the miRNA in conjunction with a nucleic acid sequence encoding the nuclease.

### TECHNICAL FIELD

The present invention relates to methods for cell-specifically regulating nuclease and relates to miRNA-responsive mRNA for use in the methods.

### BACKGROUND ART

The CRISPR/Cas9 System cleaves a gene of interest with a nuclease Cas9 protein (Cas9) and a single guide RNA (sgRNA) having 20 bases complementary to a target sequence incorporated therein (See, for example, Non Patent Literature 1). In recent years, there is a trend to use this CRISPR/Cas9 System for cell therapy. For example, by placing a Cas9 gene or an sgRNA under the control of a cancer cell-specific promoter, the CRISPR/Cas9 System is activated in a cancer-cell specific manner to induce cell death (See, for example, Non Patent Literature 2).

### CITATION LIST

### Non Patent Literatures

Non Patent Literature 1: Le Cong et al., Science 339, 819 (2013)
Non Patent Literature 2: Yuchen Liu et al. Yuchen Liu et al. 1466996882191_0 5, 2014

### SUMMARY OF INVENTION

### Technical Problems

However, since a plasmid has been used for the introduction of a Cas9 and an sgRNA into a cell with the CRISPR/Cas9 System, there has been a risk that they will be inserted in the genome, resulting in mutations. On the other hand, an introduction method using a promoter has had problems in that (1) the promoter has a low specificity (it is highly expressed in a cancer-specific manner, while it may be somewhat expressed in a normal cell); and (2) a cancer cell-specific promoter is generally weak in gene expression. An introduction method by using a gene circuit (AND gate) has been tried to solve these problems, but there have still been problems in that (1) it requires a search for a combination of suitable promoters; and (2) it requires the construction of a plasmid each time to change the promoter into another one.

### Solution to Problems

The present inventors have intensively performed research to solve the above-described problems, and as a result, they have solved the problems by using a nucleic acid sequence specifically recognized by an miRNA which has been developed by the present inventors in conjunction with a nucleic acid sequence encoding a nuclease. They have thus found a method for regulating the activity of a nuclease in response to a cell-specific miRNA.

Accordingly, the present invention provides the following:

[1] A method for cell-specifically regulating a nuclease, comprising a step of introducing an miRNA-responsive mRNA encoding the nuclease into cells, wherein the miRNA-responsive mRNA comprises:

(i) a nucleic acid sequence specifically recognized by the miRNA; and

(ii) a nucleic acid sequence corresponding to a coding region for the nuclease.

[2] The method according to [1], wherein the nucleic acid sequence specifically recognized by the miRNA is either an miR-302a-target sequence or an miR-21-target sequence.
[3] The method according to [1] or [2], wherein the nuclease is a Cas9 protein or a variant thereof, and the method further comprises a step of introducing into the group of cells an sgRNA comprising a guide sequence specifically recognized by a target gene for the nuclease.
[4] The method according to [3], for regulating the nuclease in a manner specific to undifferentiated cells.
[5] The method according to [4], wherein the undifferentiated cells are cells expressing miR-302a.
[6] The method according to any one of [1] to [5], wherein the miRNA-responsive mRNA comprises a nucleic acid sequence having (i) and (ii) linked in a 5′ to 3′ direction.
[7] An miRNA-responsive mRNA, comprising:

(i) a nucleic acid sequence specifically recognized by the miRNA; and

(ii) a nucleic acid sequence corresponding to a coding region for a nuclease.

[8] The miRNA-responsive mRNA according to [7], wherein the nucleic acid sequence specifically recognized by the miRNA is either an miR-302a-target sequence or an miR-21-target sequence.
[9] The miRNA-responsive mRNA according to [7] or [8], wherein the nuclease is a Cas9 protein or a variant thereof.
[10] A kit for cell-specifically regulating a nuclease, comprising:

the miRNA-responsive mRNA according to [9]; and

an sgRNA comprising a guide sequence specifically recognized by a target gene for the nuclease.

[11] A method for cell-specifically regulating a nuclease, comprising a step of introducing into cells

a) a trigger protein-responsive mRNA encoding the nuclease; and

b) an miRNA-responsive mRNA encoding the trigger protein,

wherein the protein-responsive mRNA encoding the nuclease a) comprises:
(ia) a nucleic acid sequence that specifically binds to the protein; and
(iia) a nucleic acid sequence corresponding to a coding region for the nuclease; and

wherein the miRNA-responsive mRNA encoding the trigger protein b) comprises:
(ib) a nucleic acid sequence specifically recognized by the miRNA; and
(iib) a nucleic acid sequence corresponding to a coding region for the trigger protein.
   [12] The method according to [11], wherein the trigger protein comprises an L7Ae protein or a derivative thereof, and the nucleic acid sequence that specifically binds to the protein comprises a K-turn sequence or a derivative thereof.
   [13] The method according to [11] or [12], wherein the nucleic acid sequence specifically recognized by the miRNA is either an miR-302a-target sequence or an miR-21-target sequence.
   [14] The method according to any one of [11] to [13], wherein the nuclease is a Cas9 protein or a variant thereof, and the method further comprises a step of introducing into the cells an sgRNA comprising a guide sequence specifically recognized by a target gene for the nuclease.
   [15] The method according to [14], for regulating the nuclease in a manner specific to undifferentiated cells.
   [16] The method according to [15], wherein the undifferentiated cells are cells expressing miR-302a.
   [17] The method according to any one of [11] to [16], wherein:

the a) protein-responsive mRNA encoding the trigger protein comprises a nucleic acid sequence having (ia) and (iia) linked in a 5′ to 3′ direction; and

the b) miRNA-responsive mRNA encoding the nuclease comprises a nucleic acid sequence having (ib) and (iib) linked in a 5′ to 3′ direction.

[18] A nuclease regulator comprising:

a) a trigger protein-responsive mRNA encoding a nuclease, comprising:
(ia) a nucleic acid sequence that specifically binds to the protein; and
(iia) a nucleic acid sequence corresponding to a coding region for the nuclease; and

b) an miRNA-responsive mRNA encoding the trigger protein comprising:
(ib) a nucleic acid sequence specifically recognized by the miRNA; and
(iib) a nucleic acid sequence corresponding to a coding region for the trigger protein.
   [19] The nuclease regulator according to [18], wherein the trigger protein comprises an L7Ae protein or a derivative thereof, and the nucleic acid sequence that specifically binds to the protein comprises a K-turn sequence or a derivative thereof.
   [20] The nuclease regulator according to [18] or [19], wherein in the miRNA-responsive mRNA encoding the trigger protein, the nucleic acid sequence specifically recognized by the miRNA is either an miR-302a-target sequence or an miR-21-target sequence.
   [21] The nuclease regulator according to any one of [18] to [20], wherein in the trigger protein-responsive mRNA, the nuclease is a Cas9 protein or a variant thereof.
   [22] A kit for cell-specifically regulating a nuclease, comprising:

the nuclease regulator according to any one of [18] to [21]; and

an sgRNA comprising a guide sequence specifically recognized by a target gene for the nuclease.

### Advantageous Effects of Invention

The method for regulating a nuclease according to the present invention has made it possible to regulate the activity of the nuclease in response to the expression of an miRNA representing the state of cells. In the method of the present invention, a nuclease can be introduced into cells with an mRNA and can be regulated cell-specifically. Therefore, from the viewpoint of safety and specificity, applications in clinical use are expected.

### BRIEF DESCRIPTION OF DRAWINGS

FIG. 1(a) is a conceptual illustration showing a construct of an miRNA-responsive mRNA encoding a nuclease used in the first embodiment of the present invention.

FIG. 1(b) is a conceptual illustration showing the reaction mechanism of an miRNA-responsive Cas9 mRNA in an microRNA (miRNA)-responsive CRISPR/Cas9 System.

FIG. 2(a) shows photographs of fluorescence images showing the knockout of fluorescent genes by an miRNA-responsive CRISPR/Cas9 mRNA introduced into HeLa cells.

FIG. 2(b) shows histograms showing the knockout of fluorescent genes by the miRNA-responsive CRISPR/Cas9 mRNA introduced into HeLa cells.

FIG. 2(c) is a graph obtained by defining as the Cas9 activity the GFP negative population in the histograms shown in FIG. 2(b).

FIG. 3 is a graph indicating that an miR-21-responsive Cas9 mRNA introduced into HeLa cells was translationally repressed by miR-21 endogenous to the HeLa cells, resulting in regulation of expression of the Cas9.

FIG. 4(a) is a graph showing the Cas9 activity determined by the EGFP activity assay indicating that an miR-302a-responsive Cas9 mRNA introduced into iPS cells was translationally repressed by miR-302a endogenous to the iPS cells.

FIG. 4(b) is a gel image showing the results of the T7E1 assay in which an miR-302a-responsive Cas9 mRNA was introduced into iPS cells. The gel image and quantitative values obtained therefrom indicate that the Cas9 activity is correlated with the GFP negative population.

FIG. 4(c) is a graph showing indels (Cas9 activity) determined from the gel image shown in FIG. 4(b).

FIG. 5(a) is a schematic view of the Cell killing system, and FIG. 5(b) is a graph showing the rate of dead cells when an miR-21-responsive Cas9 mRNA and an sgRNA targeting Alu1 were introduced into HeLa cells.

FIG. 6(a) is a graph showing the relative Cas9 activity determined by the EGFP activity assay in which an miR-302a-responsive Cas9 mRNA was introduced into mDA cells.

FIG. 6(b) is a gel image of the T7E1 assay in which an miR-302a-responsive Cas9 mRNA was introduced into mDA cells.

FIG. 6(c) is a graph showing the relative Cas9 activity determined based on the T7E1 assay.

FIG. 7(a) shows the results of evaluating the co-culture of iPS_GFP cells and HeLa_GFP cells by a change in the fluorescence intensity by the GFP knockout.

FIG. 7(b) is a graph showing the Cas9 activity calculated based on the results of FIG. 7(a).

FIG. 8(a) is a conceptual illustration showing two constructs of an miRNA-responsive mRNA encoding a trigger protein and a trigger protein-responsive mRNA encoding a nuclease, used in the second embodiment of the present invention.

FIG. 8(b) is a graph showing that the Cas9 activity was increased by miR-21 endogenous to HeLa cells by introducing a set of mRNAs in the second embodiment.

FIG. 8(c) is a graph showing that when using the two constructs of an miRNA-responsive mRNA encoding a trigger protein and a trigger protein-responsive mRNA encoding a nuclease, schematically shown in FIG. 8(a), the Cas9 activity was increased in response to the intracellular miRNA.

FIG. 9(a) shows the results of the expression levels of miR-21 each of which was measured with HeLa-EGFP cells, iPS-EGFP cells and mDA-EGFP cells.

FIG. 9(b) shows the results of the expression levels of miR-302a each of which was measured with HeLa-EGFP cells, iPS-EGFP cells and mDA-EGFP cells.

FIG. 10 shows the results of examining by the Simple Western (Wes) assay whether each of a control Cas9 mRNA, an miR-21-responsive Cas9 mRNA and an miR-302a-responsive Cas9 mRNA has an effect on the expression of a Cas9 protein by introducing each of them (also introducing an sgRNA together therewith) into HeLa cells.

FIG. 11(a) is a scheme of sequencing a PCR product, showing a sequence around the target sequence.

FIG. 11(b) shows that a mutation is introduced in the EGFP gene target region of the PCR product.

FIG. 12(a) shows the results of examining with HeLa-EGFP cells by qPCR the expression variation of a gene the functions of which are known to be regulated by miR-21.

FIG. 12(b) shows the results of examining with iPS-EGFP cells by qPCR the expression variation of a gene the functions of which are known to be regulated by miR-302a.

FIG. 13(a) shows the transfection efficiency of HeLa-EGFP cells with each of a BFP mRNA and an sgRNA.

FIG. 13(b) shows the transfection efficiency of iPS-EGFP cells with each of a BFP mRNA and an sgRNA.

FIG. 14(a) shows the Cas9 activity when “GG” was deleted from the 5′ end of an EGFP-targeting sgRNA.

FIG. 14(b) shows the results when an miR-302-responsive Cas9 mRNA was modified into a 4×miR-302-responsive Cas9 mRNA in order to reduce the leakage of the Cas9 activity.

FIG. 15(a) shows photographs of cells in which 4×miR-302-responsive Cas9 mRNAs were introduced.

FIG. 15(b) shows histograms showing the Cas9 activity in cells in which 4×miR-302-responsive Cas9 mRNAs were introduced.

FIG. 15(c) shows the result of quantitatively determining the Cas9 activity in cells in which 4×miR-302-responsive Cas9 mRNAs were introduced.

### DESCRIPTION OF EMBODIMENTS

The present invention will be described in detail below with reference to embodiments thereof but will not be limited thereto.

The present invention provides a method for cell-specifically regulating a nuclease. The method for cell-specifically regulating a nuclease was performed at least by using a nucleic acid sequence specifically recognized by an miRNA in conjunction with a nucleic acid sequence encoding the nuclease. For example, a nuclease can be cell-specifically regulated by using one mRNA in which a nucleic acid sequence specifically recognized by an miRNA and a nucleic acid sequence encoding the nuclease are included. Alternatively, a nuclease can be cell-specifically regulated by using a combination of an mRNA containing a nucleic acid sequence specifically recognized by an miRNA and another mRNA containing nucleic acid sequence encoding the nuclease. Hereinafter, the present invention will be described with reference to each of the embodiments.

### First Embodiment: OFF Switch Nuclease Regulation

According to one embodiment, the present invention is a method for cell-specifically regulating a nuclease, comprising a step of introducing an miRNA-responsive mRNA encoding the nuclease into a cell. In the present invention, “cell-specifically regulating a nuclease” refers to regulating the activity of a nuclease based on the expression state of an miRNA endogenous to a cell.

The cell in the present invention may be any cell without being particularly limited thereto. For example, the cell may be cells collected from a multicellular organism species or cells obtained by culturing an isolated cell. In particular, the cells are cells collected from a mammal (such as a human, a mouse, a monkey, a pig, a rat or the like), or cells isolated from a mammal or cells obtained by culturing a mammalian cell line. Examples of the somatic cells include epithelial cells that keratinizes (such as keratinized epidermal cells), mucosal epithelial cells (such as an epithelial cells in the tongue surface layer), exocrine gland epithelial cells (such as a mammary gland cells), hormone-secreting cells (such as adrenal medulla cells), cells for metabolism/storage (such as hepatocytes), luminal epithelial cells constituting the interface (such as type I pneumocytes), luminal epithelial cells of the closed tube (such as vascular endothelial cells), cells with cilia having transport capacity (such as airway epithelial cells), secreting cells of a extracellular matrix (such as fibroblasts), contractile cells (such as smooth muscle cells), blood and immune cells (such as T lymphocytes), cells related to sensation (such as rod cells), neurons of the autonomic nervous system (such as cholinergic neurons), sustentacular cells of the sensory organ and peripheral neurons (such as satellite cells), nerve cells and glial cells of the central nervous system (such as astroglial cells), pigment cells (such as retinal pigment epithelial cells), and progenitor cells (tissue progenitor cells) thereof.

The cell to be used in the present invention is not particularly limited in the degree of cell differentiation, the age of the animal from which a cell is collected, and the like, and whether undifferentiated progenitor cells (including somatic stem cells) or terminally differentiated mature cells can likewise be used as cells in the present invention. Examples of the undifferentiated progenitor cell include tissue stem cells (somatic stem cells) such as neural stem cells, hematopoietic stem cells, mesenchymal stem cells or dental pulp stem cells.

The cell to be used in the present invention may be a cell obtained by collecting a somatic cell and artificially manipulating it, such as a group of cells comprising an iPS cell prepared from the somatic cell, or a group of cells obtained by differentiating a pluripotent stem cell, exemplified by a ES cell and an iPS cell, which can comprise other differentiated cells than the desired cell. The cell to be used in the present invention is particularly preferably in a living state. In the present invention, “a cell is in a living state” refers to a cell having metabolic capacity maintained. The cell to be used in the present invention is a cell that does not lose its inherent properties even after introducing an miRNA responsive mRNA into the cell but can be used for subsequent applications while remaining in a living state, particularly maintaining its division potential.

For such a cell, it is known that the type and amount of the miRNA expressed in the cell is specific to the cell type. In the present invention, the activity of a nuclease can be regulated in response to an miRNA expressed in the cell by using an miRNA-responsive mRNA encoding the nuclease, as described in detail below. As used herein, “regulating the activity of a nuclease” refers to “decreasing or increasing the expression level of a nuclease to thereby decrease or increase the activity of the nuclease.

miRNA-Responsive mRNA

In the present invention, an miRNA-responsive mRNA encoding a nuclease is also referred to as an miRNA-responsive mRNA or an miRNA switch and means an mRNA comprising the following nucleic acid sequences (i) and (ii): (i) a nucleic acid sequence specifically recognized by an miRNA; and (ii) a nucleic acid sequence corresponding to a coding region for a nuclease.

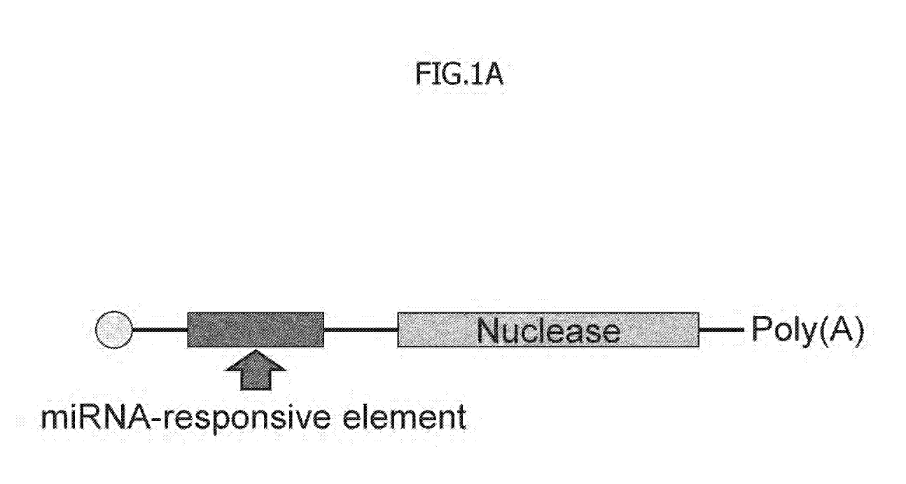

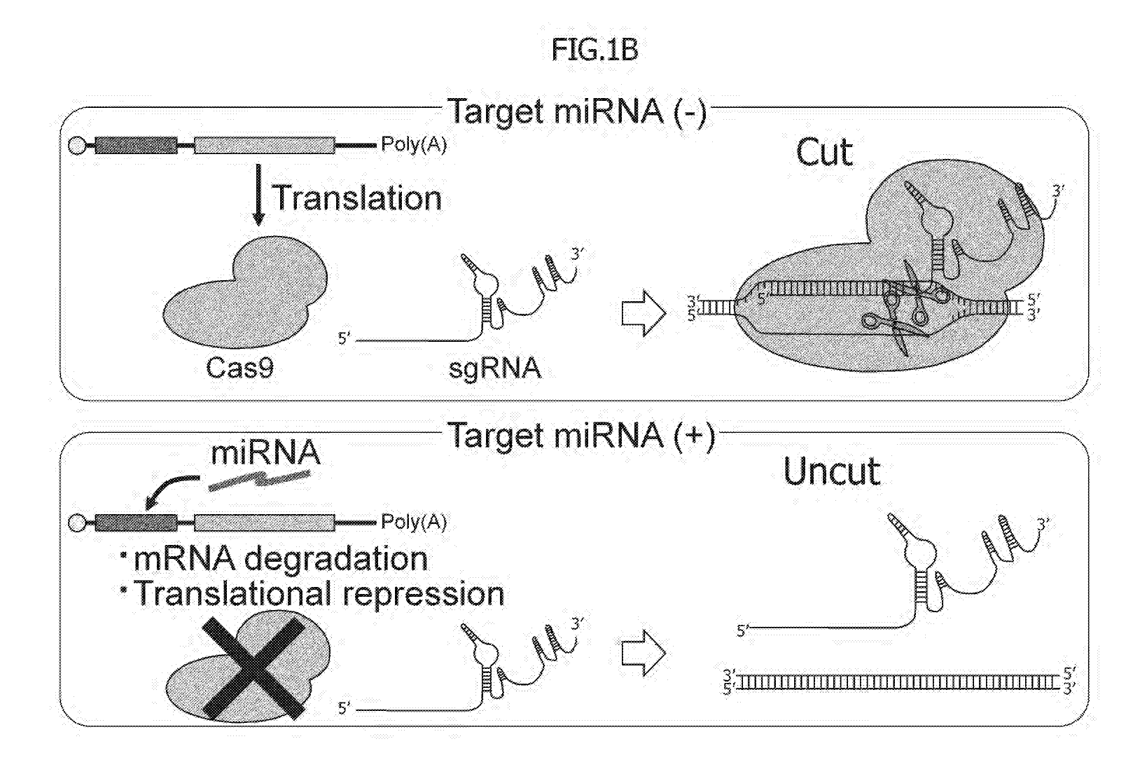

The (i) nucleic acid sequence specifically recognized by an miRNA and the (ii) nucleic acid sequence corresponding to a coding region for a nuclease are functionally linked with each other. FIG. 1(a) is an illustration schematically showing an example of an miRNA-responsive mRNA that can be used in the method according to the present invention. This miRNA-responsive mRNA has a sequence responding to the miRNA incorporated in its 5′ UTR, and has a gene encoding a nuclease inserted in its protein coding region.

In the present invention, “miRNA” is a short chain (20 to 25 bases) non-coding RNA contained in a cell that is involved in regulation of the gene expression by inhibiting the translation from an mRNA to a protein and degrading the mRNA. This miRNA functions in such a manner that the miRNA is transcribed as a single-stranded pri-miRNA capable of taking a hairpin-loop structure containing the miRNA and its complementary strand, partly cleaved into a pre-miRNA by an enzyme, referred to as Drosha, present in a nucleus, then transported outside the nucleus and further cleaved by Dicer.

As the miRNA of (i), for the purpose of regulating the activity of a nuclease in a particular cell, particularly repressing or activating the expression of the nuclease, it is possible to appropriately select an miRNA that is specifically expressed in the cell or is not specifically expressed. Examples of the miRNA that is specifically expressed include an miRNA that is expressed more highly in a particular cell by 10% or more, 20% or more, 30% or more, 40% or more, 50% or more, 60% or more, 70% or more, 80% or more, 90% or more, or more than other cells. Such an miRNA can be appropriately selected from miRNAs registered in a database (such as http://www.mirbase.org/or http://www.microrna.org/) and/or miRNAs described in the literature described in the database.

In the present invention, the nucleic acid sequence specifically recognized by an miRNA is preferably, for example, a sequence completely complementary to the miRNA. Alternatively, the nucleic acid sequence specifically recognized by an miRNA may have any mismatch (discordance) with the completely complementary sequence as long as it may be recognized by the miRNA. There may be any mismatch with the sequence completely complementary to the miRNA as long as recognition by the miRNA may normally be performed in the desired cell, and for the inherent functions in the cell in vivo, there may be a mismatch of approximately 40 to 50%. Examples of such a mismatch include, but are not particularly limited to, a mismatch of 1 base, 2 bases, 3 bases, 4 bases, 5 bases, 6 bases, 7 bases, 8 bases, 9 bases or 10 bases, or a mismatch of 1%, 5%, 10%, 20%, 30% or 40% of the overall sequence that is recognized. In addition, particularly as with the miRNA target sequence on the mRNA contained in the cell, particularly the portion other than a seed region, that is, a region on the 5′ side in the target sequence that corresponds to approximately 16 bases on the 3′ side in the miRNA may contain many mismatches, whereas the portion of the seed region may contain no mismatch or a mismatch of 1 base, 2 bases or 3 bases. Such a sequence may have any base length as long as it contains the bases to which an RISC specifically binds, and the base length is, but not particularly limited to, preferably 18 bases or more and less than 24 bases, more preferably 20 bases or more and less than 22 bases. In the present invention, the nucleic acid sequence specifically recognized by an miRNA can be appropriately determined and used by introducing an miRNA-responsive mRNA having the sequence into the desired cell and the other cells and confirming that the expression of the corresponding marker gene is repressed only in the desired cell.

As used herein, “a nucleic acid sequence corresponding to a coding region for a nuclease” of (ii) described above refers to a gene encoding a protein that is translated in a cell to function as an enzyme degrading a nucleic acid. Examples of the nucleic acid sequence corresponding to a coding region for a nuclease include, but not limited to, a gene encoding clustered regularly interspaced short palindromic repeats-associated proteins 9 (Cas9), a gene encoding a transcription activator-like effector nuclease (TALEN), a gene encoding a homing endonuclease and a gene encoding a zinc finger nuclease. For any nuclease, a variant or derivative thereof having the same function can be used. For example, the nucleic acid sequence corresponding to a coding region for a nuclease includes a gene encoding a Cas9 variant or the gene encoding a Cas9 variant-fused protein. Each of these nucleases can be also designed specifically for a target nucleic acid that is degraded by the nuclease. When the nuclease is a Cas9, an sgRNA is designed specifically for a target nucleic acid that is degraded by the nuclease. Details will be described later.

As used herein, “a nucleic acid sequence specifically recognized by an miRNA and a nucleic acid sequence corresponding to a coding region for a nuclease are functionally linked with each other” means that at least one miRNA target sequence is contained in the 5′ UTR, in the 3′ UTR of the open reading frame encoding the nuclease (including an initiation codon) and/or within the open reading frame. An miRNA-responsive mRNA preferably contains a cap structure (7-methylguanosine 5′-phosphate), an open reading frame encoding a nuclease and a poly-A tail from the 5′ end in a 5′ to 3′ direction, and contains at least one miRNA target sequence in the 5′ UTR, in the 3′ UTR, and/or in the open reading frame. The position of the miRNA target sequence in the mRNA may be in the 5′ UTR or in the 3′ UTR, or may be within the open reading frame (3′ to the initiation codon), or the miRNA target sequence may be contained in all of these regions. Therefore, the number of miRNA target sequences may be 1, 2, 3, 4, 5, 6, 7, 8 or more.

Preferably, the miRNA-responsive mRNA comprises nucleic acid sequences having (i) and (ii) linked in this order in a 5′ to 3′ direction. Any number of any types of bases may be contained between the cap structure and the miRNA target sequence as long as the base(s) do not constitute a stem structure or a three-dimensional structure. For example, the miRNA-responsive mRNA can be designed so that the number of bases between the cap structure and the miRNA target sequence is 0 to 50 bases and preferably 10 to 30 bases. Any number of any types of bases may be contained between the miRNA target sequence and the initiation codon as long as the base(s) do not constitute a stem structure or a three-dimensional structure, and the miRNA-responsive mRNA can be designed so that the number of bases between the miRNA target sequence and the initiation codon is 0 to 50 bases and preferably 10 to 30 bases.

In the present invention, an miRNA target sequence in an miRNA-responsive mRNA preferably has no AUG functioning as an initiation codon. For example, when the miRNA-responsive mRNA contains the miRNA target sequence in the 5′ UTR and AUG in the target sequence, it is preferably designed so that AUG is in frame in relation to the marker gene to be linked on the 3′ side. Alternatively, when the miRNA-responsive mRNA contains AUG in the target sequence, it is also possible to use the miRNA-responsive mRNA by converting AUG in the target sequence into GUG. Also, in order to minimize the influence of AUG in the target sequence, the location of the target sequence in the 5′ UTR can be appropriately changed. For example, the miRNA-responsive mRNA can be designed so that the number of bases between the cap structure and the AUG sequence in the target sequence is 0 to 60 bases, for example, 0 to 15 bases, 10 to 20 bases, 20 to 30 bases, 30 to 40 bases, 40 to 50 bases or 50 to 60 bases.

In the method of the present invention, one type of miRNA-responsive mRNA may be introduced into a cell, or 2, 3, 4, 5, 6, 7, 8 or more types may be introduced. When two or more types of miRNA-responsive mRNAs are introduced, for example, mRNAs that have miRNA target sites different in sequences and respond to different miRNAs can be used. By using a plurality of different miRNA-responsive mRNAs, for example, it is possible to regulate the activity of a nuclease for each of a plurality of types of cells that can be contained in a group of cells into which the miRNA-responsive mRNAs are introduced.

Introduction of miRNA-responsive mRNA In the present invention, an miRNA-responsive mRNA can be introduced into a cell in the form of an mRNA. For example, it may be introduced into a somatic cell by such a technique as lipofection and microinjection, and an RNA having 5-methylcytidine and pseudouridine (TriLink BioTechnologies, Inc.) incorporated therein may be used to repress the degradation (Warren L, (2010) Cell Stem Cell 7: 618-630). For each of uridine and cytidine, all or a part of the bases can be independently modified. When a part of the bases are modified, the modified bases can be randomly positioned at any ratio. Alternatively, the miRNA-responsive mRNA can be introduced into a cell in the form of a DNA such as a vector. It can be introduced in the same way as described above.

When introducing two or more different miRNA-responsive mRNAs, or when using an miRNA-responsive mRNA and using an mRNA as a control described below (hereinafter also referred to as a control mRNA), it is preferable to co-introduce a plurality of mRNAs into a group of cells. It is because the ratio of two or more mRNAs co-introduced in a cell is maintained in each individual cell and the ratio of the activities of proteins expressed by these mRNAs is constant in the cell population. The introduction amount varies depending on the group of cells to be introduced, the mRNA to be introduced, the introduction method and the type of introduction reagent, which can be appropriately selected by those skilled in the art to obtain the desired translation amount of a nuclease. In the present invention, a “control mRNA” refers to an mRNA having no miRNA target site. That is, the control mRNA is an RNA that is introduced into a cell and translated therein, without being affected by the expression level of any miRNA endogenous to the cell. The control mRNA can be preferably introduced into a group of cells together with an miRNA-responsive mRNA, and can function as a control for confirming and identifying the cells having the miRNA-responsive mRNA introduced therein. The introduction amount of the control mRNA can also be appropriately selected by those skilled in the art to obtain the desired translation amount.

When an miRNA specifically recognizing a target sequence is in a cell, an miRNA-responsive mRNA of interest introduced into a cell is translationally repressed to decrease the expression level of a nuclease and to thus decrease the activity of the nuclease. Therefore, the nuclease activity can be decreased specifically for the cell expressing a particular miRNA. This results in a decrease in the activity of cleaving a target gene targeted by the nuclease and thus a less effect on the function of the target gene. On the other hand, when an miRNA specifically recognizing a target sequence is not in a cell, an miRNA-responsive mRNA is not translationally repressed and a nuclease is thus expressed in the cell. As a result, in the cell, the cleavage of the target gene targeted by the nuclease proceeds to impair the function of the gene, by which the fate of the cell related to the function of the gene can be regulated. Therefore, in one example, cells that do not contain an miRNA specifically recognizing a target sequence can lead to cell death.

Next, as a more specific example, the method of the present invention when using a Cas9 protein as a nuclease will be described. According to one embodiment, the present invention comprises a step of introducing into a cell an miRNA-responsive mRNA encoding a Cas9 protein, and a step of introducing into the cell an sgRNA containing a guide sequence specifically recognized by a target sequence for the Cas9 protein. FIG. 1 (b) is an illustration schematically showing the action inside a cell when an miRNA-responsive Cas9 mRNA encoding a Cas9 protein and an sgRNA are introduced into a cell. In the absence of a target miRNA that binds to the miRNA-responsive Cas9 mRNA (Target miRNA (−)), the Cas9 protein is translated from the miRNA-responsive mRNA. Therefore, the guide strand (sgRNA) co-introduced with the miRNA-responsive mRNA forms a complex together with the Cas9 protein and genome editing, specifically the cleavage of the target DNA is performed. In the presence of the target miRNA (Target miRNA (+)), the target miRNA binds to the 5 ‘UTR of the miRNA-responsive Cas9 mRNA, resulting in translation repression and degradation of an mRNA switch, and the translation of the Cas9 protein from the miRNA-responsive Cas9 mRNA is repressed. Therefore, since the Cas9/sgRNA complex is not formed, and genome editing, specifically the cleavage of the target DNA is not performed.

The miRNA-responsive mRNA encoding a Cas9 protein can be designed as described above. The miRNA-responsive mRNA encoding a Cas9 protein is hereinafter also referred to as an miRNA-responsive Cas9 mRNA. Examples of the miRNA include miR-302a and miR-21. The miR-302a is known as an miRNA endogenous to a pluripotent stem cell such as an iPS cell and an ES cell. Therefore, by introducing an miR-302a-responsive mRNA encoding the Cas9 protein into a group of cells comprising pluripotent stem cells such as iPS cells and differentiated cells, it is possible to repress the expression of the Cas9 protein specifically for the pluripotent stem cell and keep the target gene function of an sgRNA without being impaired, whereas it is possible in the differentiated cell to allow the Cas9 protein to function and cleave the target gene. That is, the activity of the Cas9 protein can be regulated depending on the degree of cell differentiation or the state of cell initialization (reprogramming). On the other hand, miR-21 is known to be specifically expressed in a HeLa cell. These sequences are shown in Table 1 below. An example of the miR target sequences specifically recognized by each of miR-302a and miR-21 is shown in Table 2.

TABLE 1

| | | Sequence |
|---|---|---|
| RNA name | Sequence (5′ → 3′) | ID No. |
| miR-302a | ACUUAAACGUGGAUGUACUUGCU | 1 |
| miR-21 | UAGCUUAUCAGACUGAUGUUGA | 2 |

TABLE 2

| | | Sequence |
|---|---|---|
| RNA name | Sequence (5′ → 3′) | ID No. |
| Tg miR-302a | AGCAAGUACAUCCACGUUUAAGU | 3 |
| Tg miR-21 | UCAACAUCAGUCUGAUAAGCUA | 4 |

Specific examples of the miR-302a-responsive Cas9 mRNA that has an miR-302a target sequence and responds to miR-302a endogenous to a cell to repress the expression of a Cas9 protein, and the miR-21-responsive Cas9 mRNA that has an miR-21 target sequence and responds to miR-21 endogenous to a cell to repress the expression of the Cas9 protein include, but is not limited to, sequences shown in Table 3 below. In the sequences in Table 3, each of the underlined parts and the double-underlined parts represents an miRNA target sequence, AUG represents an initiation codon, and each of the dotted-underlined parts represents a 3 ‘UTR, respectively.

TABLE 3A

| RNA | | Sequence |
|---|---|---|
| name | Sequence(5′ -> 3′) | ID No. |
| miR- | GGUUCCUUAAUCGCGGAUCCAGCAAGUACAUCCACGUUUAAGUAGAUCCACCG | 5 |
| 302a | 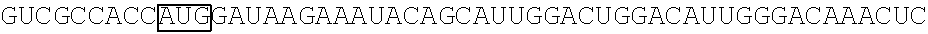 | |
| respon- | CGUGGGAUGGGCCGUGAUUACAGACGAAUACAAAGUGCCUUCAAAGAAGUUCA | |
| sive | AGGUGCUGGGCAACACCGAUAGACACAGCAUCAAGAAAAAUCUGAUUGGAGCC | |
| Cas | CUGCUGUUCGACUCCGGCGAGACAGCUGAAGCAACUCGGCUGAAAAGAACUGC | |
| 9 mRNA | UCGGAGAAGGUAUACCCGCCGAAAGAAUAGGAUCUGCUACCUGCAGGAGAUUU | |
| | UCAGCAACGAAAUGGCCAAGGUGGACGAUAGUUUCUUUCACCGCCUGGAGGAA | |
| | UCAUUCCUGGUCGAGGAAGAUAAGAAACACGAGCGGCAUCCCAUCUUUGGCAA | |
| | CAUUGUGGACGAGGUCGCUUAUCACGAAAAGUACCCUACCAUCUAUCAUCUGA | |
| | GGAAGAAACUGGUGGACUCCACAGAUAAAGCAGACCUGCGCCUGAUCUAUCUG | |
| | GCCCUGGCUCACAUGAUUAAGUUCCGGGGCCAUUUUCUGAUCGAGGGGGAUCU | |
| | GAACCCAGACAAUUCUGAUGUGGACAAGCUGUUCAUCCAGCUGGUCCAGACAU | |
| | ACAAUCAGCUGUUUGAGGAAAACCCCAUUAAUGCAUCUGGCGUGGACGCAAAA | |
| | GCCAUCCUGAGUGCCAGACUGUCUAAGAGUCGGAGACUGGAGAACCUGAUCGC | |
| | UCAGCUGCCAGGGGAAAAGAAAAACGGCCUGUUUGGGAAUCUGAUUGCACUGU | |
| | CACUGGGACUGACUCCCAACUUCAAGAGCAAUUUUGAUCUGGCCGAGGACGCU | |
| | AAACUGCAGCUGUCCAAGGACACCUAUGACGAUGACCUGGAUAACCUGCUGGC | |
| | UCAGAUCGGGGAUCAGUACGCAGACCUGUUCCUGGCCGCUAAGAAUCUGUCUG | |
| | ACGCCAUCCUGCUGAGUGAUAUUCUGCGCGUGAACACCGAGAUUACAAAAGCC | |
| | CCCCUGUCAGCUAGCAUGAUCAAGAGAUAUGACGAGCACCAUCAGGAUCUGAC | |
| | CCUGCUGAAGGCUCUGGUGAGGCAGCAGCUGCCUGAGAAGUACAAGGAAAUCU | |
| | UCUUUGAUCAGUCUAAGAACGGAUACGCCGGCUAUAUUGACGGCGGGGCUAGU | |
| | CAGGAGGAGUUCUACAAGUUUAUCAAACCCAUUCUGGAGAAGAUGGAUGGCAC | |
| | AGAGGAACUGCUGGUGAAACUGAAUCGGGAAGACCUGCUGAGGAAGCAGCGCA | |
| | CUUUUGAUAACGGAAGCAUCCCUCACCAGAUUCAUCUGGGAGAGCUGCACGCA | |
| | AUCCUGAGGCGCCAGGAAGACUUCUACCCAUUUCUGAAGGAUAACAGGGAGAA | |
| | GAUCGAAAAAAUUCUGACAUUCCGCAUCCCCUACUAUGUGGGCCCUCUGGCAA | |
| | GAGGCAACAGCCGGUUUGCCUGGAUGACUCGCAAAUCUGAGGAAACAAUCACU | |
| | CCCUGGAACUUCGAGGAAGUGGUCGAUAAGGGCGCUUCCGCACAGUCUUUCAU | |
| | UGAGCGGAUGACAAACUUCGACAAGAACCUGCCAAACGAAAAAGUGCUGCCCA | |
| | AGCACUCUCUGCUGUACGAGUAUUUCACAGUCUAUAACGAACUGACUAAGGUG | |
| | AAAUACGUCACCGAGGGGAUGAGAAAGCCUGCCUUCCUGAGUGGAGAACAGAA | |
| | GAAAGCUAUCGUGGACCUGCUGUUUAAAACCAAUAGGAAGGUGACAGUCAAGC | |
| | AGCUGAAAGAGGACUAUUUCAAGAAAAUUGAAUGUUUCGAUUCUGUGGAGAUC | |
| | AGUGGCGUCGAAGACAGGUUUAACGCCUCCCUGGGGACCUACCACGAUCUGCU | |
| | GAAGAUCAUUAAGGAUAAAGACUUCCUGGACAACGAGGAAAAUGAGGAUAUCC | |
| | UGGAAGACAUUGUGCUGACCCUGACACUGUUUGAGGAUAGGGAAAUGAUCGAG | |
| | GAACGCCUGAAGACCUAUGCCCAUCUGUUCGAUGACAAAGUGAUGAAACAGCU | |
| | GAAGCGACGGAGAUACACAGGAUGGGGCCGACUGUCUCGGAAGCUGAUCAAUG | |
| | GGAUUCGCGACAAACAGAGUGGAAAGACCAUCCUGGACUUUCUGAAAUCAGAU | |
| | GGCUUCGCCAACCGGAACUUCAUGCAGCUGAUUCACGAUGACAGCCUGACAUU | |
| | CAAAGAGGAUAUCCAGAAGGCACAGGUGUCCGGGCAGGGAGACUCUCUGCACG | |
| | AGCAUAUCGCAAACCUGGCCGGCAGCCCUGCCAUCAAGAAAGGGAUUCUGGAG | |
| | ACCGUGAAGGUGGUGGACGAGCUGGUGAAAGUCAUGGGAAGACAUAAGCCAGA | |
| | AAACAUCGUGAUUGAGAUGGCCAGGGAAAAUCAGACCACACAGAAAGGCCAGA | |
| | AGAACUCAAGGGAGCGCAUGAAAAGAAUCGAGGAAGGAAUUAAGGAACUGGGC | |
| | AGCCAGAUCCUGAAAGAGCACCCCGUGGAAAACACACAGCUGCAGAAUGAGAA | |
| | GCUGUAUCUGUACUAUCUGCAGAAUGGACGCGAUAUGUACGUGGACCAGGAGC | |
| | UGGAUAUUAACCGACUGUCCGAUUACGACGUGGAUCAUAUCGUCCCACAGUCA | |
| | UUCCUGAAAGAUGACAGCAUUGACAAUAAGGUGCUGACCCGCUCUGACAAAAA | |
| | CCGAGGCAAGAGUGAUAAUGUCCCCUCAGAGGAAGUGGUCAAGAAAAUGAAGA | |
| | ACUACUGGAGGCAGCUGCUGAAUGCCAAACUGAUCACACAGCGAAAGUUUGAU | |

TABLE 3B

| | | |
|---|---|---|
| | AACCUGACUAAAGCUGAGCGGGGAGGCCUGAGUGAACUGGACAAAGCAGGCUU | |
| | CAUUAAGCGACAGCUGGUGGAGACACGGCAGAUCACAAAGCACGUCGCCCAGA | |
| | UUCUGGAUUCAAGAAUGAACACUAAGUACGAUGAGAAUGACAAACUGAUCAGA | |
| | GAAGUGAAGGUCAUUACCCUGAAGUCAAAACUGGUGAGCGACUUUCGGAAAGA | |
| | UUUCCAGUUUUAUAAGGUCAGAGAGAUCAACAACUACCACCAUGCUCAUGACG | |
| | CAUACCUGAACGCAGUGGUCGGCACAGCCCUGAUUAAGAAAUACCCUAAACUG | |
| | GAGUCCGAGUUCGUGUACGGGGACUAUAAGGUGUACGAUGUCAGAAAAAUGAU | |
| | CGCCAAGUCUGAGCAGGAAAUUGGCAAAGCCACUGCUAAGUAUUUCUUUUACA | |
| | GUAACAUCAUGAAUUUCUUUAAGACUGAGAUCACCCUGGCAAAUGGGGAAAUC | |
| | CGAAAGCGGCCACUGAUUGAGACUAACGGCGAGACAGGAGAAAUCGUGUGGGA | |
| | CAAAGGAAGAGAUUUUGCUACCGUGAGGAAGGUCCUGAGCAUGCCCCAAGUGA | |
| | AUAUUGUCAAGAAAACAGAGGUGGAGACUGGGGGAUUCAGUAAGGAAUCAAUU | |
| | CUGCCUAAACGCAACUCCGAUAAGCUGAUCGCCCGAAAGAAAGACUGGGACCC | |
| | CAAGAAGUAUGGCGGGUUCGACUCCCCAACUGUGGCUUACUCUGUCCUGGUGG | |
| | UCGCAAAGGUGGAGAAGGGAAAAAGCAAGAAACUGAAAUCCGUCAAGGAACUG | |
| | CUGGGCAUCACCAUUAUGGAGCGCAGCUCCUUCGAAAAGAAUCCUAUCGAUUU | |
| | UCUGGAGGCCAAAGGCUAUAAGGAAGUGAAGAAAGACCUGAUCAUCAAGCUGC | |
| | CAAAGUACUCACUGUUUGAGCUGGAAAACGGGAGAAAGAGGAUGCUGGCAAGC | |
| | GCCGGGGAGCUGCAGAAAGGAAAUGAACUGGCCCUGCCCUCCAAGUACGUGAA | |
| | CUUCCUGUAUCUGGCUAGCCACUACGAGAAGCUGAAAGGGUCCCCUGAGGAUA | |
| | ACGAACAGAAACAGCUGUUUGUGGAGCAGCACAAGCAUUAUCUGGACGAGAUC | |
| | AUUGAACAGAUUAGCGAGUUCUCCAAAAGAGUGAUCCUGGCUGACGCAAAUCU | |
| | GGAUAAGGUCCUGAGCGCAUACAACAAACACCGGGAUAAGCCAAUCAGAGAGC | |
| | AGGCCGAAAAUAUCAUUCAUCUGUUCACUCUGACCAACCUGGGAGCCCCCGCA | |
| | GCCUUCAAGUAUUUUGACACUACCAUCGAUCGCAAACGAUACACAAGCACUAA | |
| | GGAGGUGCUGGACGCUACCCUGAUUCAUCAGAGCAUUACUGGCCUGUAUGAAA | |
| | CAAGGAUUGACCUGUCUCAGCUGGGCGGCGACUCCGGAGCUGACCCCAAGAAG | |
| | 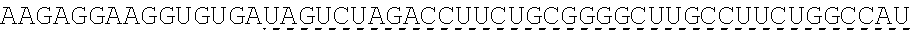 | |
| | 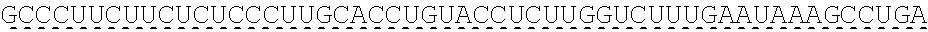 | |
| | 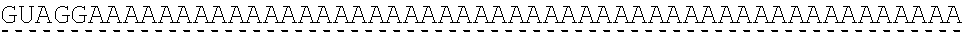 | |
| | 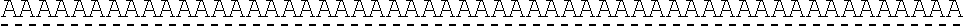 | |
| | 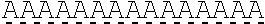 | |
| miR-21 | GGUUCCUUAAUCGCGGAUCCUCAACAUCAGUCUGAUAAGCUAAGAUCACACCG | 6 |
| respon- | 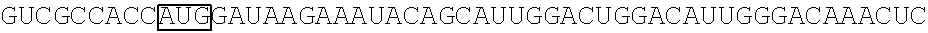 | |
| sive | CGUGGGAUGGGCCGUGAUUACAGACGAAUACAAAGUGCCUUCAAAGAAGUUCA | |
| Cas | AGGUGCUGGGCAACACCGAUAGACACAGCAUCAAGAAAAAUCUGAUUGGAGCC | |
| 9 | CUGCUGUUCGACUCCGGCGAGACAGCUGAAGCAACUCGGCUGAAAAGAACUGC | |
| mRNA | UCGGAGAAGGUAUACCCGCCGAAAGAAUAGGAUCUGCUACCUGCAGGAGAUUU | |
| | UCAGCAACGAAAUGGCCAAGGUGGACGAUAGUUUCUUUCACCGCCUGGAGGAA | |
| | UCAUUCCUGGUCGAGGAAGAUAAGAAACACGAGCGGCAUCCCAUCUUUGGCAA | |
| | CAUUGUGGACGAGGUCGCUUAUCACGAAAAGUACCCUACCAUCUAUCAUCUGA | |
| | GGAAGAAACUGGUGGACUCCACAGAUAAAGCAGACCUGCGCCUGAUCUAUCUG | |
| | GCCCUGGCUCACAUGAUUAAGUUCCGGGGCCAUUUUCUGAUCGAGGGGGAUCU | |
| | GAACCCAGACAAUUCUGAUGUGGACAAGCUGUUCAUCCAGCUGGUCCAGACAU | |
| | ACAAUCAGCUGUUUGAGGAAAACCCCAUUAAUGCAUCUGGCGUGGACGCAAAA | |
| | GCCAUCCUGAGUGCCAGACUGUCUAAGAGUCGGAGACUGGAGAACCUGAUCGC | |
| | UCAGCUGCCAGGGGAAAAGAAAAACGGCCUGUUUGGGAAUCUGAUUGCACUGU | |
| | CACUGGGACUGACUCCCAACUUCAAGAGCAAUUUUGAUCUGGCCGAGGACGCU | |
| | AAACUGCAGCUGUCCAAGGACACCUAUGACGAUGACCUGGAUAACCUGCUGGC | |
| | UCAGAUCGGGGAUCAGUACGCAGACCUGUUCCUGGCCGCUAAGAAUCUGUCUG | |
| | ACGCCAUCCUGCUGAGUGAUAUUCUGCGCGUGAACACCGAGAUUACAAAAGCC | |
| | CCCCUGUCAGCUAGCAUGAUCAAGAGAUAUGACGAGCACCAUCAGGAUCUGAC | |
| | CCUGCUGAAGGCUCUGGUGAGGCAGCAGCUGCCUGAGAAGUACAAGGAAAUCU | |
| | UCUUUGAUCAGUCUAAGAACGGAUACGCCGGCUAUAUUGACGGCGGGGCUAGU | |
| | CAGGAGGAGUUCUACAAGUUUAUCAAACCCAUUCUGGAGAAGAUGGAUGGCAC | |
| | AGAGGAACUGCUGGUGAAACUGAAUCGGGAAGACCUGCUGAGGAAGCAGCGCA | |
| | CUUUUGAUAACGGAAGCAUCCCUCACCAGAUUCAUCUGGGAGAGCUGCACGCA | |
| | AUCCUGAGGCGCCAGGAAGACUUCUACCCAUUUCUGAAGGAUAACAGGGAGAA | |
| | GAUCGAAAAAAUUCUGACAUUCCGCAUCCCCUACUAUGUGGGCCCUCUGGCAA | |

TABLE 3C
GAGGCAACAGCCGGUUUGCCUGGAUGACUCGCAAAUCUGAGGAAACAAU
CACUCCCUGGAACUUCGAGGAAGUGGUCGAUAAGGGCGCUUCCGCACAG
UCUUUCAUUGAGCGGAUGACAAACUUCGACAAGAACCUGCCAAACGAAA
AAGUGCUGCCCAAGCACUCUCUGCUGUACGAGUAUUUCACAGUCUAUAA
CGAACUGACUAAGGUGAAAUACGUCACCGAGGGGAUGAGAAAGCCUGCC
UUCCUGAGUGGAGAACAGAAGAAAGCUAUCGUGGACCUGCUGUUUAAAA
CCAAUAGGAAGGUGACAGUCAAGCAGCUGAAAGAGGACUAUUUCAAGAA
AAUUGAAUGUUUCGAUUCUGUGGAGAUCAGUGGCGUCGAAGACAGGUUU
AACGCCUCCCUGGGGACCUACCACGAUCUGCUGAAGAUCAUUAAGGAUA
AAGACUUCCUGGACAACGAGGAAAAUGAGGAUAUCCUGGAAGACAUUGU
GCUGACCCUGACACUGUUUGAGGAUAGGGAAAUGAUCGAGGAACGCCUG
AAGACCUAUGCCCAUCUGUUCGAUGACAAAGUGAUGAAACAGCUGAAGC
GACGGAGAUACACAGGAUGGGGCCGACUGUCUCGGAAGCUGAUCAAUGG
GAUUCGCGACAAACAGAGUGGAAAGACCAUCCUGGACUUUCUGAAAUCA
GAUGGCUUCGCCAACCGGAACUUCAUGCAGCUGAUUCACGAUGACAGCC
UGACAUUCAAAGAGGAUAUCCAGAAGGCACAGGUGUCCGGGCAGGGAGA
CUCUCUGCACGAGCAUAUCGCAAACCUGGCCGGCAGCCCUGCCAUCAAG
AAAGGGAUUCUGCAGACCGUGAAGGUGGUGGACGAGCUGGUGAAAGUCA
UGGGAAGACAUAAGCCAGAAAACAUCGUGAUUGAGAUGGCCAGGGAAAA
UCAGACCACACAGAAAGGCCAGAAGAACUCAAGGGAGCGCAUGAAAAGA
AUCGAGGAAGGAAUUAAGGAACUGGGCAGCCAGAUCCUGAAAGAGCACC
CCGUGGAAAACACACAGCUGCAGAAUGAGAAGCUGUAUCUGUACUAUCU
GCAGAAUGGACGCGAUAUGUACGUGGACCAGGAGCUGGAUAUUAACCGA
CUGUCCGAUUACGACGUGGAUCAUAUCGUCCCACAGUCAUUCCUGAAAG
AUGACAGCAUUGACAAUAAGGUGCUGACCCGCUCUGACAAAAACCGAGG
CAAGAGUGAUAAUGUCCCCUCAGAGGAAGUGGUCAAGAAAAUGAAGAAC
UACUGGAGGCAGCUGCUGAAUGCCAAACUGAUCACACAGCGAAAGUUUG
AUAACCUGACUAAAGCUGAGCGGGGAGGCCUGAGUGAACUGGACAAAGC
AGGCUUCAUUAAGCGACAGCUGGUGGAGACACGGCAGAUCACAAAGCAC
GUCGCCCAGAUUCUGGAUUCAAGAAUGAACACUAAGUACGAUGAGAAUG
ACAAACUGAUCAGAGAAGUGAAGGUCAUUACCCUGAAGUCAAAACUGGU
GAGCGACUUUCGGAAAGAUUUCCAGUUUUAUAAGGUCAGAGAGAUCAAC
AACUACCACCAUGCUCAUGACGCAUACCUGAACGCAGUGGUCGGCACAG
CCCUGAUUAAGAAAUACCCUAAACUGGAGUCCGAGUUCGUGUACGGGGA
CUAUAAGGUGUACGAUGUCAGAAAAAUGAUCGCCAAGUCUGAGCAGGAA
AUUGGCAAAGCCACUGCUAAGUAUUUCUUUUACAGUAACAUCAUGAAUU
UCUUUAAGACUGAGAUCACCCUGGCAAAUGGGGAAAUCCGAAAGCGGCC
ACUGAUUGAGACUAACGGCGAGACAGGAGAAAUCGUGUGGGACAAAGGA
AGAGAUUUUGCUACCGUGAGGAAGGUCCUGAGCAUGCCCCAAGUGAAUA
UUGUCAAGAAAACAGAGGUGCAGACUGGGGGAUUCAGUAAGGAAUCAAU
UCUGCCUAAACGCAACUCCGAUAAGCUGAUCGCCCGAAAGAAAGACUGG
GACCCCAAGAAGUAUGGCGGGUUCGACUCCCCAACUGUGGCUUACUCUG
UCCUGGUGGUCGCAAAGGUGGAGAAGGGAAAAAGCAAGAAACUGAAAUC
CGUCAAGGAACUGCUGGGCAUCACCAUUAUGGAGCGCAGCUCCUUCGAA
AAGAAUCCUAUCGAUUUUCUGGAGGCCAAAGGCUAUAAGGAAGUGAAGA
AAGACCUGAUCAUCAAGCUGCCAAAGUACUCACUGUUUGAGCUGGAAAA
CGGGAGAAAGAGGAUGCUGGCAAGCGCCGGGGAGCUGCAGAAAGGAAAU
GAACUGGCCCUGCCCUCCAAGUACGUGAACUUCCUGUAUCUGGCUAGCC
ACUACGAGAAGCUGAAAGGGUCCCCUGAGGAUAACGAACAGAAACAGCU
GUUUGUGGAGCAGCACAAGCAUUAUCUGGACGAGAUCAUUGAACAGAUU
AGCGAGUUCUCCAAAAGAGUGAUCCUGGCUGACGCAAAUCUGGAUAAGG
UCCUGAGCGCAUACAACAAACACCGGGAUAAGCCAAUCAGAGAGCAGGC
CGAAAAUAUCAUUCAUCUGUUCACUCUGACCAACCUGGGAGCCCCCGCA
GCCUUCAAGUAUUUUGACACUACCAUCGAUCGCAAACGAUACACAAGCA
CUAAGGAGGUGCUGGACGCUACCCUGAUUCAUCAGAGCAUUACUGGCCU
GUAUGAAACAAGGAUUGACCUGUCUCAGCUGGGCGGCGACUCCGGAGCU

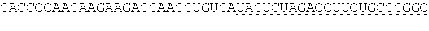

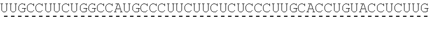

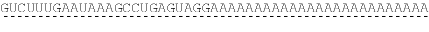

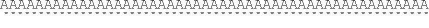

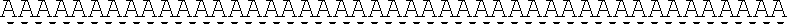

TABLE 3D

| | | |
|---|---|---|
| 4 x miR- | GGUUCCUUAAUCGCGGAUCCAGCAAGUACAUCCACGUUUAAGUAGCAAGUACA | 7 |
| 30 | UCCACGUUUAAGUAGCAAGUACAUCCACGUUUAAGUAGCAAGUACAUCCACGU | |
| 2a | 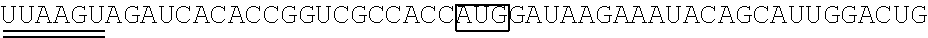 | |
| respon- | GACAUUGGGACAAACUCCGUGGGAUGGGCCGUGAUUACAGACGAAUACAAAGU | |
| sive | GCCUUCAAAGAAGUUCAAGGUGCUGGGCAACACCGAUAGACACAGCAUCAAGA | |
| Cas 9 | AAAAUCUGAUUGGAGCCCUGCUGUUCGACUCCGGCGAGACAGCUGAAGCAACU | |
| mRNA | CGGCUGAAAAGAACUGCUCGGAGAAGGUAUACCCGCCGAAAGAAUAGGAUCUG | |
| | CUACCUGCAGGAGAUUUUCAGCAACGAAAUGGCCAAGGUGGACGAUAGUUUCU | |
| | UUCACCGCCUGGAGGAAUCAUUCCUGGUCGAGGAAGAUAAGAAACACGAGCGG | |
| | CAUCCCAUCUUUGGCAACAUUGUGGACGAGGUCGCUUAUCACGAAAAGUACCC | |
| | UACCAUCUAUCAUCUGAGGAAGAAACUGGUGGACUCCACAGAUAAAGCAGACC | |
| | UGCGCCUGAUCUAUCUGGCCCUGGCUCACAUGAUUAAGUUCCGGGGCCAUUUU | |
| | CUGAUCGAGGGGGAUCUGAACCCAGACAAUUCUGAUGUGGACAAGCUGUUCAU | |
| | CCAGCUGGUCCAGACAUACAAUCAGCUGUUUGAGGAAAACCCCAUUAAUGCAU | |
| | CUGGCGUGGACGCAAAAGCCAUCCUGAGUGCCAGACUGUCUAAGAGUCGGAGA | |
| | CUGGAGAACCUGAUCGCUCAGCUGCCAGGGGAAAAGAAAAACGGCCUGUUUGG | |
| | GAAUCUGAUUGCACUGUCACUGGGACUGACUCCCAACUUCAAGAGCAAUUUUG | |
| | AUCUGGCCGAGGACGCUAAACUGCAGCUGUCCAAGGACACCUAUGACGAUGAC | |
| | CUGGAUAACCUGCUGGCUCAGAUCGGGGAUCAGUACGCAGACCUGUUCCUGGC | |
| | CGCUAAGAAUCUGUCUGACGCCAUCCUGCUGAGUGAUAUUCUGCGCGUGAACA | |
| | CCGAGAUUACAAAAGCCCCCCUGUCAGCUAGCAUGAUCAAGAGAUAUGACGAG | |
| | CACCAUCAGGAUCUGACCCUGCUGAAGGCUCUGGUGAGGCAGCAGCUGCCUGA | |
| | GAAGUACAAGGAAAUCUUCUUUGAUCAGUCUAAGAACGGAUACGCCGGCUAUA | |
| | UUGACGGCGGGGCUAGUCAGGAGGAGUUCUACAAGUUUAUCAAACCCAUUCUG | |
| | GAGAAGAUGGAUGGCACAGAGGAACUGCUGGUGAAACUGAAUCGGGAAGACCU | |
| | GCUGAGGAAGCAGCGCACUUUUGAUAACGGAAGCAUCCCUCACCAGAUUCAUC | |
| | UGGGAGAGCUGCACGCAAUCCUGAGGCGCCAGGAAGACUUCUACCCAUUUCUG | |
| | AAGGAUAACAGGGAGAAGAUCGAAAAAAUUCUGACAUUCCGCAUCCCCUACUA | |
| | UGUGGGCCCUCUGGCAAGAGGCAACAGCCGGUUUGCCUGGAUGACUCGCAAAU | |
| | CUGAGGAAACAAUCACUCCCUGGAACUUCGAGGAAGUGGUCGAUAAGGGCGCU | |
| | UCCGCACAGUCUUUCAUUGAGCGGAUGACAAACUUCGACAAGAACCUGCCAAA | |
| | CGAAAAAGUGCUGCCCAAGCACUCUCUGCUGUACGAGUAUUUCACAGUCUAUA | |
| | ACGAACUGACUAAGGUGAAAUACGUCACCGAGGGGAUGAGAAAGCCUGCCUUC | |
| | CUGAGUGGAGAACAGAAGAAAGCUAUCGUGGACCUGCUGUUUAAAACCAAUAG | |
| | GAAGGUGACAGUCAAGCAGCUGAAAGAGGACUAUUUCAAGAAAAUUGAAUGUU | |
| | UCGAUUCUGUGGAGAUCAGUGGCGUCGAAGACAGGUUUAACGCCUCCCUGGG | |
| | GACCUACCACGAUCUGCUGAAGAUCAUUAAGGAUAAAGACUUCCUGGACAACG | |
| | AGGAAAAUGAGGAUAUCCUGGAAGACAUUGUGCUGACCCUGACACUGUUUGAG | |
| | GAUAGGGAAAUGAUCGAGGAACGCCUGAAGACCUAUGCCCAUCUGUUCGAUGA | |
| | CAAAGUGAUGAAACAGCUGAAGCGACGGAGAUACACAGGAUGGGGCCGACUGU | |
| | CUCGGAAGCUGAUCAAUGGGAUUCGCGACAAACAGAGUGGAAAGACCAUCCUG | |
| | GACUUUCUGAAAUCAGAUGGCUUCGCCAACCGGAACUUCAUGCAGCUGAUUCA | |
| | CGAUGACAGCCUGACAUUCAAAGAGGAUAUCCAGAAGGCACAGGUGUCCGGGC | |
| | AGGGAGACUCUCUGCACGAGCAUAUCGCAAACCUGGCCGGCAGCCCUGCCAUC | |
| | AAGAAAGGGAUUCUGCAGACCGUGAAGGUGGUGGACGAGCUGGUGAAAGUCAU | |
| | GGGAAGACAUAAGCCAGAAAACAUCGUGAUUGAGAUGGCCAGGGAAAAUCAGA | |
| | CCACACAGAAAGGCCAGAAGAACUCAAGGGAGCGCAUGAAAAGAAUCGAGGAA | |
| | GGAAUUAAGGAACUGGGCAGCCAGAUCCUGAAAGAGCACCCCGUGGAAAACAC | |
| | ACAGCUGCAGAAUGAGAAGCUGUAUCUGUACUAUCUGCAGAAUGGACGCGAUA | |
| | UGUACGUGGACCAGGAGCUGGAUAUUAACCGACUGUCCGAUUACGACGUGGAU | |
| | CAUAUCGUCCCACAGUCAUUCCUGAAAGAUGACAGCAUUGACAAUAAGGUGCU | |
| | GACCCGCUCUGACAAAAACCGAGGCAAGAGUGAUAAUGUCCCCUCAGAGGAAG | |
| | UGGUCAAGAAAAUGAAGAACUACUGGAGGCAGCUGCUGAAUGCCAAACUGAUC | |
| | ACACAGCGAAAGUUUGAUAACCUGACUAAAGCUGAGCGGGGAGGCCUGAGUGA | |
| | ACUGGACAAAGCAGGCUUCAUUAAGCGACAGCUGGUGGAGACACGGCAGAUCA | |
| | CAAAGCACGUCGCCCAGAUUCUGGAUUCAAGAAUGAACACUAAGUACGAUGAG | |
| | AAUGACAAACUGAUCAGAGAAGUGAAGGUCAUUACCCUGAAGUCAAAACUGGU | |
| | GAGCGACUUUCGGAAAGAUUUCCAGUUUUAUAAGGUCAGAGAGAUCAACAACU | |

TABLE 3E
ACCACCAUGCUCAUGACGCAUACCUGAACGCAGUGGUCGGCACAGCCCU
GAUUAAGAAAUACCCUAAACUGGAGUCCGAGUUCGUGUACGGGGACUAU
AAGGUGUACGAUGUCAGAAAAAUGAUCGCCAAGUCUGAGCAGGAAAUUG
GCAAAGCCACUGCUAAGUAUUUCUUUUACAGUAACAUCAUGAAUUUCUU
UAAGACUGAGAUCACCCUGGCAAAUGGGGAAAUCCGAAAGCGGCCACUG
AUUGAGACUAACGGCGAGACAGGAGAAAUCGUGUGGGACAAAGGAAGAG
AUUUUGCUACCGUGAGGAAGGUCCUGAGCAUGCCCCAAGUGAAUAUUGU
CAAGAAAACAGAGGUGCAGACUGGGGGAUUCAGUAAGGAAUCAAUUCUG
CCUAAACGCAACUCCGAUAAGCUGAUCGCCCGAAAGAAAGACUGGGACC
CCAAGAAGUAUGGCGGGUUCGACUCCCCAACUGUGGCUUACUCUGUCCU
GGUGGUCGCAAAGGUGGAGAAGGGAAAAAGCAAGAAACUGAAAUCCGUC
AAGGAACUGCUGGGCAUCACCAUUAUGGAGCGCAGCUCCUUCGAAAAGA
AUCCUAUCGAUUUUCUGGAGGCCAAAGGCUAUAAGGAAGUGAAGAAAGA
CCUGAUCAUCAAGCUGCCAAAGUACUCACUGUUUGAGCUGGAAAACGGG
AGAAAGAGGAUGCUGGCAAGCGCCGGGGAGCUGCAGAAAGGAAAUGAAC
UGGCCCUGCCCUCCAAGUACGUGAACUUCCUGUAUCUGGCUAGCCACUA
CGAGAAGCUGAAAGGGUCCCCUGAGGAUAACGAACAGAAACAGCUGUUU
GUGGAGCAGCACAAGCAUUAUCUGGACGAGAUCAUUGAACAGAUUAGCG
AGUUCUCCAAAAGAGUGAUCCUGGCUGACGCAAAUCUGGAUAAGGUCCU
GAGCGCAUACAACAAACACCGGGAUAAGCCAAUCAGAGAGCAGGCCGAA
AAUAUCAUUCAUCUGUUCACUCUGACCAACCUGGGAGCCCCCGCAGCCU
UCAAGUAUUUUGACACUACCAUCGAUCGCAAACGAUACACAAGCACUAA
GGAGGUGCUGGACGCUACCCUGAUUCAUCAGAGCAUUACUGGCCUGUAU
GAAACAAGGAUUGACCUGUCUCAGCUGGGCGGCGACUCCGGAGCUGACC

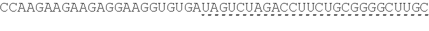

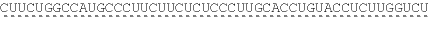

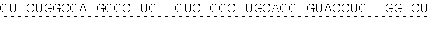

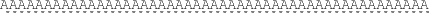

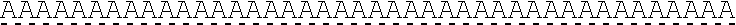

An sgRNA can be designed so as to have, a nucleotide sequence of approximately 20 bases, for example, approximately 18 to 22 bases that specifically recognizes a Cas9 target gene, incorporated in the vicinity of the 5’ end. The nucleotide sequence that specifically recognizes the Cas9 target gene is preferably a sequence completely complementary to the target gene. Alternatively, the nucleotide sequence that specifically recognizes the Cas9 target gene may have any mismatch (discordance) with the completely complementary sequence as long as the target gene may be recognized. The mismatch with the sequence completely complementary to the target gene may be similar to that defined for the miRNA and its target sequence. Also, there may be a sequence of approximately 1 to 5 bases 5′ to the nucleotide sequence specifically recognizing the Cas9 target gene. Furthermore, the sequence on the 3′ side in the nucleotide sequence specifically recognizing the target gene is not limited to particular sequences shown in Table 4 below, and may be a sequence of an sgRNA that is known to function in the CRISPR/Cas9 System, as an example of which a sequence having the stem-loop 2 of Tetraloop modified at the 3′ end is known. However, it is not particularly limited as long as it can cleave the target gene together with the Cas9.

For example, the sgRNAs shown in Table 4 below can be used in the present invention, but the sgRNA that can be used in the method of the present invention is not limited thereto. In the sequences of Table 4, each of the underlined parts represents a sequence complementary to the Cas9 target gene. In the table, SEQ ID NO: 8 is an sgRNA targeting DMD (Duchenne muscular dystrophy) gene, SEQ ID NO: 9 is an sgRNA targeting an Alu1 repeated sequence, and SEQ ID NOs: 10 and 11 are sgRNAs targeting an EGFP gene.

TABLE 4

| | | Sequence |
|---|---|---|
| RNA name | Sequence (5′ → 3′) | ID No. |
| sgRNA_DMD | GGUAUCUUACAGGAACUCCGUUUUAG | 8 |
| | AGCUAGAAAUAGCAAGUUAAAAUAAG | |
| | GCUAGUCCGUUAUCAACUUGAAAAAG | |
| | UGGCACCGAGUCGGUGCUUU | |
| sgRNA_Alu1 | GGGCCUGUAAUCCCAGCACUUUGUUU | 9 |
| | UAGAGCUAGAAAUAGCAAGUUAAAAU | |
| | AAGGCUAGUCCGUUAUCAACUUGAAA | |
| | AAGUGGCACCGAGUCGGUGCUUU | |
| sgRNA_EGFP | GGGGGCACGGGCAGCUUGCCGGGUUU | 10 |
| | UAGAGCUAGAAAUAGCAAGUUAAAAU | |
| | AAGGCUAGUCCGUUAUCAACUUGAAA | |
| | AAGUGGCACCGAGUCGGUGCUUU | |
| sgRNA_EGFP(2) | GGGCACGGGCAGCUUGCCGGGUUUUA | 11 |
| | GAGCUAGAAAUAGCAAGUUAAAAUAA | |
| | GGCUAGUCCGUUAUCAACUUGAAAAA | |
| | GUGGCACCGAGUCGGUGCUUU | |

Examples of the target sequence that can be used to cause cell death by Cas9, for example, cell-specifically include, but is not limited to, the following Alu1 repeated sequence as well as a repeated sequence such as Telomere or (AC)n.

The step of introducing into a cell the miRNA-responsive Cas9 mRNA and the sgRNA designed as described above can be performed by the method described above. Each introduction amount can be appropriately determined by those skilled in the art so that the desired Cas9 will exert its function, and is not limited.

A kit can be prepared by combining a desired miRNA-responsive Cas9 mRNA and an sgRNA that specifically recognizes a Cas9 target gene sequence. By using this, for example, the Cas9 activity can be repressed specifically for a cell expressing a particular miRNA, and can be maintained only in a cell that do not express the particular miRNA. As an example, the Cas9 activity can repressed specifically for an undifferentiated cell expressing miR-302a, and can be maintained in a differentiated cell that hardly expresses miR-302a. By combining it with the existing ON switch, genome editing can be also performed specifically for a cell expressing a particular miRNA.

### Second Embodiment: ON Switch Nuclease Regulation

According to the second embodiment, the present invention is a method for cell-specifically regulating a nuclease, comprising a step of introducing into a cell a) a trigger protein-responsive mRNA encoding the nuclease; and b) an miRNA-responsive mRNA encoding the trigger protein. In the present invention, “cell-specifically regulating a nuclease” refers to regulating the activity of a nuclease based on the expression state of an miRNA endogenous to a cell.

In this embodiment, the definition of cells is the same as in the first embodiment, and the description thereof will thus be omitted herein. In this embodiment, the activity of a nuclease can be regulated in response to an miRNA expressed in a cell by using a) a trigger protein-responsive mRNA encoding the nuclease; and b) an miRNA-responsive mRNA encoding the trigger protein, as described in detail below. In this embodiment, “regulating the activity of a nuclease” refers to “increasing the expression level of a nuclease to thereby increase the activity of the nuclease”.

a) Trigger Protein-Responsive mRNA Encoding Nuclease

In this embodiment, the trigger protein-responsive mRNA encoding a nuclease means an mRNA comprising the following nucleic acid sequences (ia) and (iia): (ia) a nucleic acid sequence that specifically binds to the trigger protein; and (iia) a nucleic acid sequence corresponding to a coding region for the nuclease.

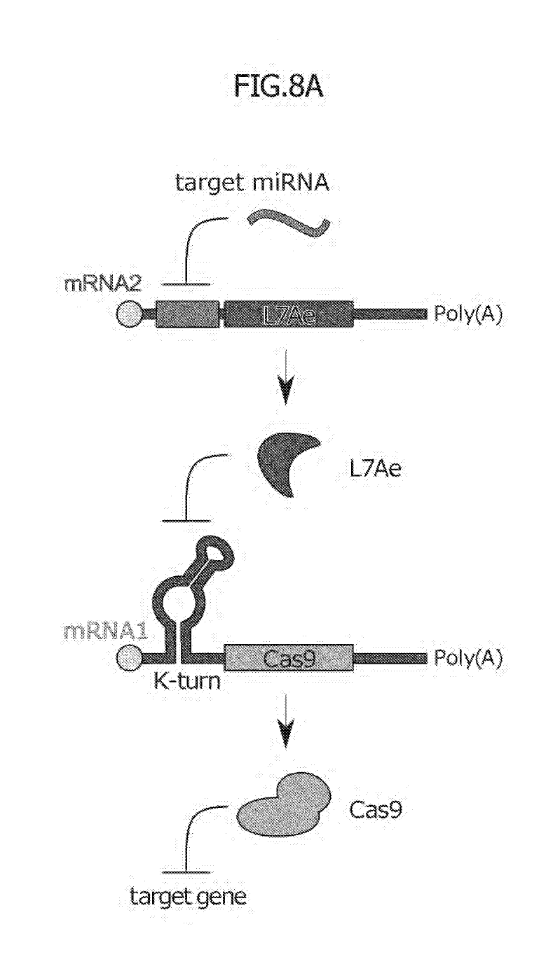

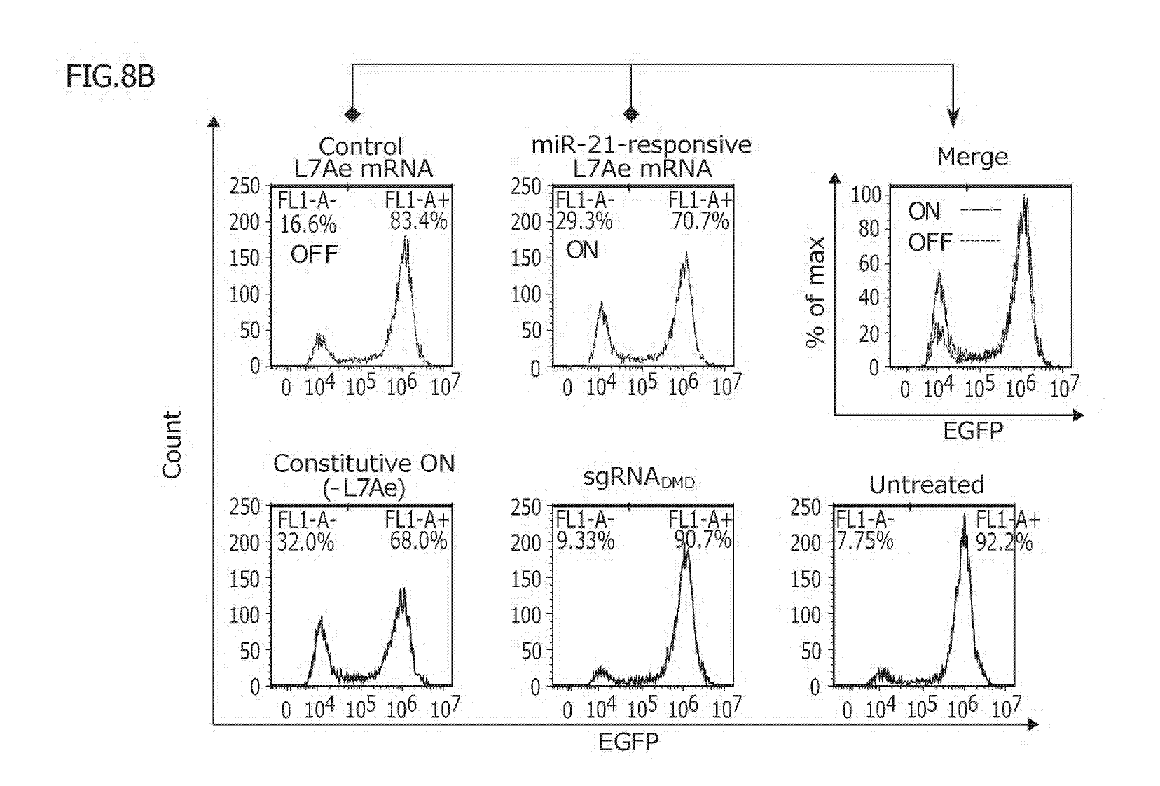

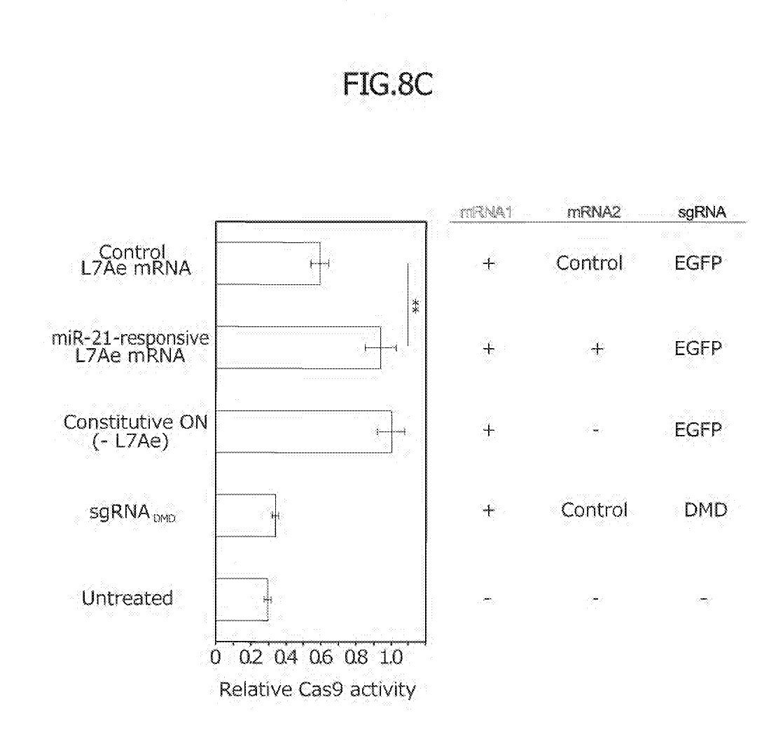

The (ia) nucleic acid sequence that specifically binds to the trigger protein and the (iia) nucleic acid sequence corresponding to a coding region for the nuclease are functionally linked with each other. FIG. 8 (a) schematically shows mRNA1, that is, an example of the trigger protein-responsive mRNA which can be used in the method according to the present invention. This trigger protein-responsive mRNA has a sequence responding to the trigger protein incorporated in its 5′ UTR, and has a gene encoding the nuclease inserted in its protein coding region.

The (ia) nucleic acid sequence that specifically binds to a trigger protein is an RNA comprising a sequence that forms an RNA-protein binding motif. As used herein, an “RNA comprising a sequence that forms an RNA-protein binding motif” refers to an RNA part contained in the RNA-protein binding motif in a natural or known RNA-protein complex, or an RNA part contained in an artificial RNA-protein binding motif obtained by an in vitro selection method. Therefore, the trigger protein comprises a protein part contained in an RNA-protein binding motif in a natural or known RNA-protein complex.

The sequence that forms a natural RNA-Protein binding motif is usually composed of approximately 5 to 30 bases, and it is known to form a specific bond with a protein having a particular amino acid sequence noncovalently, that is, via hydrogen bonding. The sequence that forms such a natural RNA-protein binding motif can be obtained by appropriately selecting the motif causing a desired structural change, from Tables 5 and 6 below, and the database available on the website: http://gibk26.bse.kyutech.acjp/j ouhou/image/dna-protein/RNA/RNA.html. The RNA-protein binding motif preferably used in this embodiment is a motif that has been already subjected to structural analysis by X-ray crystallography or structural analysis by NMR, or a motif the three-dimensional structure of which can be estimated from the three-dimensional structure of the homologous protein which has been subjected to structural analysis. In addition, it is preferably a motif in which the protein specifically recognizes the secondary structure and the nucleotide sequence of the RNA.

TABLE 5

| Name of RNA | Name of protein | Kd | Publication |
|---|---|---|---|
| 5S RNA (Xenopus laevis | 5R1 | 0.64 ± 0.10 nM | Nat Struct Biol. 1998 July; 5(7):543-6 |
| oocyte) | | | |
| 5S RNA (Xenopus laevis | 5R2 | 0.35 ± 0.03 nM | Nat Struct Biol. 1998 July; 5(7):543-6 |
| oocyte) | | | |
| dsRNA | B2 | 1.4 ± 0.13 nM | Nat Struct Mol Biol. 2005 November; |
| | | | 12(11):952-7 |
| RNA splicing motif with | Fox-1 | 0.49 nM at 150 | EMBO J. 2006 Jan. 11; 25(1):163-73. |
| UGCAUGU element | | mM salt | |
| TGE | GLD-1 | 9.2 ± 2 nM | J Mol Biol. 2005 Feb. 11; 346(1):91-104. |
| sodB | Hfq | 1.8 nM | EMBO J. 2004 Jan. 28; 23(2):396-405. |
| RyhB (siRNA) | Hfq | 1500 nM | Ann Rev Microbiol. 2004; 58:303-28 |
| mRNA | HuD | 0.7 ± 0.02 nM | Nat Struct Biol. 2001 February; 8(2):141-5 |
| S domain of 7S RNA | human SRP19 | | RNA. 2005 July; 11(7):1043-50. Epub 2005 |
| | | | May 31 |
| Large subunit of SRP RNA | human SRP19 | 2 nM | Nat Struct Biol. 2001 June; 8(6):515-20 |
| 23S rRNA | L1 | | Nat Struct Biol. 2003 February; 10(2):104-8 |
| 23S rRNA | L11 | | Nat Struct Biol. 2000 October; 7(10):834-7 |
| 5S rRNA | L18 | | Biochem J. 2002 May 1; 363(Pt 3):553-61 |
| 23S rRNA | L20 | 13 ± 2 nM | J Biol Chem. 2003 Sep. 19; 278(38):36522-30. |
| Own mRNA site1 | L20 | 88 ± 23 nM | J Biol Chem. 2003 Sep. 19; 278(38):36522-30. |
| Own mRNA site2 | L20 | 63 ± 23 nM | Mol Microbiol. 2005 June; 56(6):1441-56 |
| 23S rRNA | L23 | | J Biomol NMR. 2003 June; 26(2):131-7 |
| 5S rRNA | L25 | | EMBO J. 1999 Nov. 15; 18(22):6508-21 |
| Own mRNA | L30 | | Nat Struct Biol. 1999 December; 6(12):1081-3. |
| mRNA | LicT | | EMBO J. 2002 Apr. 15; 21(8):1987-97 |
| Own mRNA | MS2 coat | 39 ± 5 nM | FEBS J. 2006 April; 273(7):1463-75 |
| Stem-loop RNA motif | Nova-2 | | Cell. 2000 Feb. 4; 100(3):323-32 |
| SL2 | Nucleocapsid | 110 ± 50 nM | J Mol Biol. 2000 Aug. 11; 301(2):491-511 |
| Pre-rRNA | Nucleolin | | EMBO J. 2000 Dec. 15; 19(24):6870-81 |
| | p19 | 0.17 ± 0.02 nM | Cell. 2003 Dec. 26; 115(7):799-811 |
| Box C/D | L7Ae | 0.9 ± 0.2 nM | RNA. 2005 August; 11(8):1192-200. |

TABLE 6

| Name of RNA | Name of protein | Kd | | Publication |
|---|---|---|---|---|
| siRNA with the characteristic | PAZ(PiWi Argonaut and | | | Nat Struct Biol. 2003 December; 10(12):1026-32. |
| two-base 3' overhangs | Zwille) | | | |
| dsRNA | Rnase III | | | Cell. 2006 Jan. 27; 124(2):355-66 |
| HIV-1 RRE (IIB) | RR1-38 | 3-8 | nM | Nat Struct Biol. 1998 July; 5(7):543-6 |
| Own mRNA | S15 | 5 | nM | EMBO J. 2003 Apr. 15; 22(8):1898-908 |
| 16S rRNA | S15 | 6 | nM | Nat Struct Biol. 2000 April; 7(4):273-277. |
| Own mRNA | S15 | 43 | nM | EMBO J. 2003 Apr. 15; 22(8):1898-908 |
| 16S rRNA | S4 | 6.5 μM in 4° C., | | J Biol Chem. 1979 Mar 25; 254(6):1775-7 |
| | | 1.7 nM in 42° C. | | |
| 16S rRNA | S4 | 18 | μM | J Biol Chem. 1979 Mar. 25; 254(6):1775-7 |
| 16S rRNA | S8 | 26 ± 7 nM | | J Mol Biol. 2001 Aug. 10; 311(2):311-24 |
| mRNA | S8 | 200 | nM | RNA. 2004 June; 10(6):954-64 |
| mRNA | SacY | 1400 | nM | EMBO J. 1997 Aug. 15; 16(16)5019-29 |
| SnRNA | Sm | | | Cold Spring Harb Symp Quant Biol. 2006; 71:313-20. |
| tmRNA | SmpB | 21 ± 7 nM | | J Biochem (Tokyo). 2005 December; 138(6):729-39 |
| TD3 of tmRNA | SmpB | 650 | nM | J Biochem (Tokyo). 2005 December; 138(6):729-39 |
| U1 snRNA | snRNP U1A | 0.032 ± 0.007 nM | | Nat Struct Biol. 2000 October; 7(10):834-7 |
| | | (salt dependence) | | |
| S domain of 7S RNA | SRP54 | 500 | nM | RNA. 2005 July; 11(7):1043-50. |
| TAR | Tat | 200-800 | nM | Nucleic Acids Res. 1996 Oct. 15; 24(20):3974-81 |
| BIV TAR | Tat | 1.3 nM or 8 nM or 60 nM | | Mol Cell. 2000 November; 6(5):1067-76 |
| | | (Changed depending on | | |
| | | difference in Mg) | | |
| tRNA^{Thr} | ThrRS | 500 | nM | Nat Struct Biol. 2002 May; 9(5):343-7 |
| thrS mRNA operator | ThrRS | 10 | nM | Trends Genet. 2003 March; 19(3):155-61 |
| Single stranded mRNA | TIS11d | | | Nat Struct Mol Biol. 2004 March; 11(3):257-64. |
| PSTVd | Virp1 | 500 | nM | Nucleic Acids Res. 2003 Oct. 1; 31(19):5534-43 |
| RNA hairpin; Smaug | Vts1p | 30 | nM | Nat Struct Mol Biol. 2006 February; 13(2):177-8. |
| recognition element (SRE) | | | | |
| λ BoxB | λ N | 90 | nM | Cell. 1998 Apr. 17; 93(2):289-99 |

An RNA containing the sequence that forms an artificial RNA-protein binding motif is an RNA part contained in an RNA-protein binding motif in an artificially designed RNA-protein complex. The nucleotide sequence of such an RNA is usually contained of approximately 10 to 80 bases, and it is designed so as to form a specific bond with a particular amino acid sequence of a particular protein noncovalently, that is, via hydrogen bonding. Examples of the RNA containing the sequence that forms such an artificial RNA-protein binding motif include an RNA aptamer that specifically binds to a particular protein. The RNA aptamer that specifically binds to a desired target protein can be obtained, for example, by an evolutionary engineering technique known as an in vitro selection method or a SELEX method. The trigger protein herein is a protein to which the RNA aptamer binds. For example, the RNA sequences listed in Table 7 below are known and these can also be used as sequences that form the RNA-protein binding motif of the present invention.

TABLE 7

| Name of | Name of | | |
|---|---|---|---|
| RNA | protein | Kd | Publication |
| Rev aptamer 5 | Rev | 190 nM | RNA. 2005 December; |
| | | | 11(12):1848-57 |
| Aptamer | p50 | 5.4 ± | Proc Natl Acad Sci U.S.A. 2003 |
| | | 2.2 nM | Aug. 5; 100(16):9268-73. |
| BMV Gag | BMV Gag | 20 nM | RNA. 2005 December; |
| aptamer | | | 11(12):1848-57 |
| BMV Gag | CCMV Gag | 260 nM | RNA. 2005 December; |
| aptamer | | | 11(12):1848-57 |
| CCMV Gag | CCMV Gag | 280 nM | RNA. 2005 December; |
| aptamer | | | 11(12):1848-57 |
| CCMV Gag | BMV Gag | 480 nM | RNA. 2005 December; |
| aptamer | | | 11(12):1848-57 |

In this embodiment, the sequence that forms an RNA-protein binding motif has preferably a dissociation constant Kd for the corresponding trigger protein of approximately 0.1 nM to approximately 1 μM.

In addition to the sequences themselves that form these RNA-protein binding motifs, the sequence of the present invention also includes variants of such sequences. As used herein, the variant refers to a variant having a dissociation constant Kd higher by 10%, 20%, 30%, 40% or 50% or more, or Kd not more than 10%, 20%, 30%, 40% or 50% for the protein that specifically binds to the sequence that forms an RNA-protein binding motif. Such a variant can be appropriately selected and used as long as it can form an RNA-protein complex. The nucleotide sequence of such a variant may also be such a nucleotide sequence that can hybridize under stringent conditions with a nucleic acid (complementary strand) having a sequence complementary to the sequence (normal strand) that forms the RNA-protein binding motif. The stringent conditions can be determined based on the melting temperature (Tm) of the nucleic acid to be bound, as taught by Berger and Kimmel (1987, “Guide to Molecular Cloning Techniques”, Methods in Enzymology, Vol. 152, Academic Press, San Diego Calif.). For example, the washing conditions after hybridization usually can include conditions of approximately “1×SSC, 0.1% SDS, 37° C.”. The complementary strand is preferably a strand that remains hybridized with the normal strand of interest even when washed under such conditions. Examples of the washing conditions under which the hybridized state between the normal strand and the complementary strand thereto is maintained after washing include, but are not particularly limited to, approximately “0.5×SSC, 0.1% SDS, 42° C.” as more stringent hybridization conditions, and “0.1×SSC, 0.1% SDS, 65° C.” as still more stringent hybridization conditions. Specifically, the nucleotide sequence of such a variant contains a nucleotide sequence having a sequence identity of at least 90%, preferably at least 95%, 96%, 97%, 98% or 99% with the RNA sequence contained in the RNA-protein binding motif described above. Such a variant can retain constant binding with a protein that specifically binds to a sequence that forms the RNA-protein binding motif, and can contribute to the formation of an RNA-protein complex.

Specific examples of the sequence that forms an RNA-protein binding motif according to this embodiment include boxC motif (5′-GGCGUGAUGAGC-3′) (SEQ ID NO: 40), kink-loop (SEQ ID NO: 41) and kink-loop 2 (SEQ ID NO: 42) shown in Table 8 below, each of which is a sequence to which L7Ae (Moore T et al., Structure VOL. 12, pp. 807-818 (2004)) binds.

TABLE 8

| | | Sequence |
|---|---|---|
| RNA name | Sequence (5′ → 3′) | ID No. |
| boxC motif | GGCGUGAUGA GC | 40 |
| kink-loop | AGAUCCGGGU GUGAACGGUG | 41 |
| | AUCACCCGAG AUCC | |
| kink-loop2 | AGAUCCGGAC GUACGUGUGA | 42 |
| | ACGGUGAUCA CGUACGCCGA | |
| | GAUCC | |

Other specific examples include MS2 stem loop motif which is a sequence to which an MS2 coat protein specifically binds (22:Keryer-Bibens C, Barreau C, Osborne HB (2008) Tethering of proteins to RNAs by bacteriophage proteins. Biol Cell 100:125-138), and Fr 15 which is a sequence to which a Bacillus ribosomal protein S15 binds (24:Batey RT, Williamson JR (1996) Interaction of the Bacillus stearothermophilus ribosomal protein S15 with 16S rRNA: I. Defining the minimal RNA site. J Mol Biol 261:536-549).

Further specific examples include a sequence to be bound by threonyl-tRNA synthetase, an enzyme that is involved in aminoacylation and is known to have feedback inhibition activity of inhibiting translation by binding its own mRNA (Cell (Cambridge, Mass.) v. 97, pp. 371-381 (1999)): 5′-GGCGUAUGUGAUCUUUCGUGUGGGUCACCACUGCGCC-3′ (SEQ ID NO: 43) and variants thereof. Still further examples include a nucleotide sequence that forms an RNA-protein binding motif derived from the Bcl-2 family CED-9 which is an endogenous protein specific to a cancer cell, R9-2: 5′-GGGUGCUUCGAGCGUAGGAAGAAAGCCGGGGGCUGCAGAUAAUGUAUAGC-3′ (SEQ ID NO: 44) and variants thereof; and a nucleotide sequence derived from an aptamer of an RNA sequence that binds to NF-kappaB and variants thereof.

Next, as used herein, the “nucleic acid sequence corresponding to a coding region for a nuclease” of (iia) refers to a gene encoding a protein that is translated in a cell to function as an enzyme degrading a nucleic acid, and has the same meaning as that described in the first embodiment, and the description thereof will thus be omitted herein.

In the present invention, “a nucleic acid sequence that specifically binds to a trigger protein and a nucleic acid sequence corresponding to a coding region for a nuclease are functionally linked with each other” means that at least one nucleic acid sequence that specifically binds to the trigger protein is contained in the 5′ UTR, in the 3′ UTR of the open reading frame encoding the nuclease (including an initiation codon), and/or within the open reading frame. A trigger protein-responsive mRNA encoding the nuclease preferably comprises a cap structure (7-methylguanosine 5′-phosphate), an open reading frame encoding the nuclease and a poly-A tail from the 5′ end in a 5′ to 3′ direction, and contains at least one nucleic acid sequence that specifically binds to the trigger protein in the 5′ UTR, in the 3′ UTR, and/or within the open reading frame. The position in the mRNA of the nucleic acid sequence that specifically binds to the trigger protein may be in the 5′ UTR or in the 3′ UTR, or may be within the open reading frame (3′ to the initiation codon), or the nucleic acid sequence that specifically binds to the trigger protein may be contained in all of these regions. Therefore, the number of nucleic acid sequence that specifically binds to the trigger protein may be 1, 2, 3, 4, 5, 6, 7, 8 or more.

Preferably, the trigger protein-responsive mRNA encoding the nuclease comprises nucleic acid sequences (ia) and (iia) linked in this order in the 5′ to 3′ direction. Any number of any type of base may be contained between the cap structure and the nucleic acid sequence that specifically binds to the trigger protein as long as the one or more bases do not constitute a stem structure or a three-dimensional structure. For example, the trigger protein-responsive mRNA can be designed so that the number of base(s) between the cap structure and the nucleic acid sequence that specifically binds to the trigger protein is 0 to 50 bases and preferably 10 to 30 bases. Any number of any types of bases may be contained between the nucleic acid sequence that specifically binds to the trigger protein and the initiation codon as long as the base(s) do not constitute a stem structure or a three-dimensional structure. The trigger protein-responsive mRNA can be designed so that the number of bases between the nucleic acid sequence that specifically binds to the trigger protein and the initiation codon is 0 to 50 bases and preferably 10 to 30 bases.

In the present invention, the nucleic acid sequence that specifically binds to the trigger protein in the trigger protein-responsive mRNA preferably has no AUG functioning as an initiation codon. For example, when the trigger protein-responsive mRNA contains in the 5′ UTR the nucleic acid sequence that specifically binds to the trigger protein and contains AUG in the nucleic acid sequence, it is preferably designed so that AUG is in frame in relation to the marker gene to be linked on 3′ side. Alternatively, when the nucleic acid sequence that specifically binds to the trigger protein comprises AUG, it is also possible to use the trigger protein-responsive mRNA by converting, into GUG, AUG in the nucleic acid sequence that specifically binds to the trigger protein. Also, in order to minimize the influence of AUG in the nucleic acid sequence that specifically binds to the trigger protein, the location of the nucleic acid sequence that specifically binds to the trigger protein in the 5′ UTR can be appropriately changed. For example, the trigger protein-responsive mRNA can be designed so that the number of bases between the cap structure and the AUG sequence in the nucleic acid sequence that specifically binds to the trigger protein is 0 to 60 bases, for example, 0 to 15 bases, 10 to 20 bases, 20 to 30 bases, 30 to 40 bases, 40 to 50 bases or 50 to 60 bases.

b) miRNA-Responsive mRNA Encoding Trigger Protein

In the this embodiment, an miRNA-responsive mRNA encoding a trigger protein is also referred to as an miRNA-responsive mRNA or an miRNA switch and means an mRNA comprising the following nucleic acid sequences (ib) and (iib): (ib) a nucleic acid sequence specifically recognized by the miRNA; and (iib) a nucleic acid sequence corresponding to a coding region for the trigger protein.

The (ib) nucleic acid sequence specifically recognized by the miRNA and the (iib) nucleic acid sequence corresponding to a coding region for the trigger protein are functionally linked with each other. FIG. 8 (a) schematically shows mRNA2, that is, an example of the miRNA-responsive mRNA which can be used in the method according to this embodiment. This miRNA-responsive mRNA has a sequence responding to the miRNA incorporated in its 5′ UTR, and has a gene encoding a trigger protein inserted in its protein coding region.

The definition of “an miRNA” in the b) miRNA-responsive mRNA encoding a trigger protein, and the definition of “a nucleic acid sequence specifically recognized by an miRNA” are the same as that described in the first embodiment, and description thereof will thus be omitted herein. Also, in this embodiment, (ib) a nucleic acid sequence specifically recognized by an miRNA is also referred to as an miRNA target sequence.

The (iib) nucleic acid sequence corresponding to a coding region for a trigger protein is a coding sequence for a trigger protein. The trigger protein is determined in relation to the (ia) nucleic acid sequence that specifically binds to the trigger protein, and can be designed by selecting a combination in which the RNA sequence of (ia) and the (iib) trigger protein are specifically bound. For example, when the (ia) nucleic acid sequence that specifically binds to the trigger protein is boxC motif (SEQ ID NO: 40), kink-loop (SEQ ID NO: 41) and kink-loop 2 (SEQ ID NO: 42), the trigger protein is L7Ae (Moore T et al., Structure Vol. 12, pp. 807-818 (2004)). Other corresponding trigger proteins can be used for the (ia) nucleic acid sequence that specifically binds to the trigger protein exemplified above.

In the present invention, “(ib) a nucleic acid sequence specifically recognized by an miRNA (miRNA target sequence)” and “(iib) a nucleic acid sequence corresponding to a coding region for a trigger protein” are functionally linked with each other” means that at least one miRNA target sequence is contained in the 5′ UTR, in the 3′ UTR of the open reading frame encoding the trigger protein (including an initiation codon), and/or within the open reading frame. An miRNA-responsive mRNA encoding the trigger protein preferably comprises a cap structure (7-methylguanosine 5′-phosphate), an open reading frame encoding the trigger protein and a poly-A tail from the 5′ end in the 5′ to 3′ direction, and comprises at least one miRNA target sequence in the 5′ UTR, in the 3′ UTR, and/or within the open reading frame. The position of the miRNA target sequence in the mRNA may be in the 5′ UTR or in the 3′ UTR, or may be within the open reading frame (3′ to the initiation codon), or the miRNA target sequence may be contained in all of these regions. Therefore, the number of miRNA target sequences may be 1, 2, 3, 4, 5, 6, 7, 8 or more.

Preferably, the miRNA-responsive mRNA encoding the trigger protein comprises nucleic acid sequences (ib) and (iib) linked in this order in a 5′ to 3′ direction. The designing of the nucleic acid sequences (ib) and (iib), the number of bases between the nucleic acid sequence and the cap structure, the number and location of nucleic acid sequence (ib) and the same sequence as that of the initiation codon may be the same as that in the first embodiment or those in the designing of the trigger protein-responsive mRNA encoding a nuclease a).

A trigger protein-responsive mRNA encoding a nuclease a) and an miRNA-responsive mRNA encoding the trigger protein b) can be constructed by any conventional general engineering technique as long as it can be designed as described above. These two types of mRNAs function as a set of two. For introduction of cells, it is also possible to design two or more sets of mRNAs which are different in the miRNA target sequence, the trigger protein and the nuclease from each other. The set of mRNAs a) and b) is preferably co-introduced into a cell. At this time, a control mRNA can also be co-introduced as the same manner as in the first embodiment.

Next, regulation of a nuclease by such a set of mRNAs a) and b) will be described. When the miRNA that specifically recognizes the target sequence is present in a cell, the miRNA-responsive mRNA introduced into the cell is translationally repressed, resulting in a decrease in the expression level of the trigger protein. In the presence of the trigger protein, it binds to the trigger protein-responsive mRNA, which is translationally repressed, but when the expression level of the trigger protein decreases, the translation amount of the trigger protein-responsive mRNA conversely increases, which can promote the translation of the nuclease and thereby enhance the nuclease activity. As a result, the cleavage activity of a target gene which is a target of the nuclease is also enhanced, and the target gene is cleaved, resulting in the loss of its function. On the other hand, when the miRNA that specifically recognizes the target sequence is absent in a cell, the miRNA-responsive mRNA is not translationally repressed and the trigger protein is thus expressed in the cell. Then, the trigger protein translationally represses the trigger protein-responsive mRNA, resulting in a decrease in the expression level of the nuclease. As a result, the probability that the nuclease will cleave the target gene is decreased and the function of the target gene is maintained.

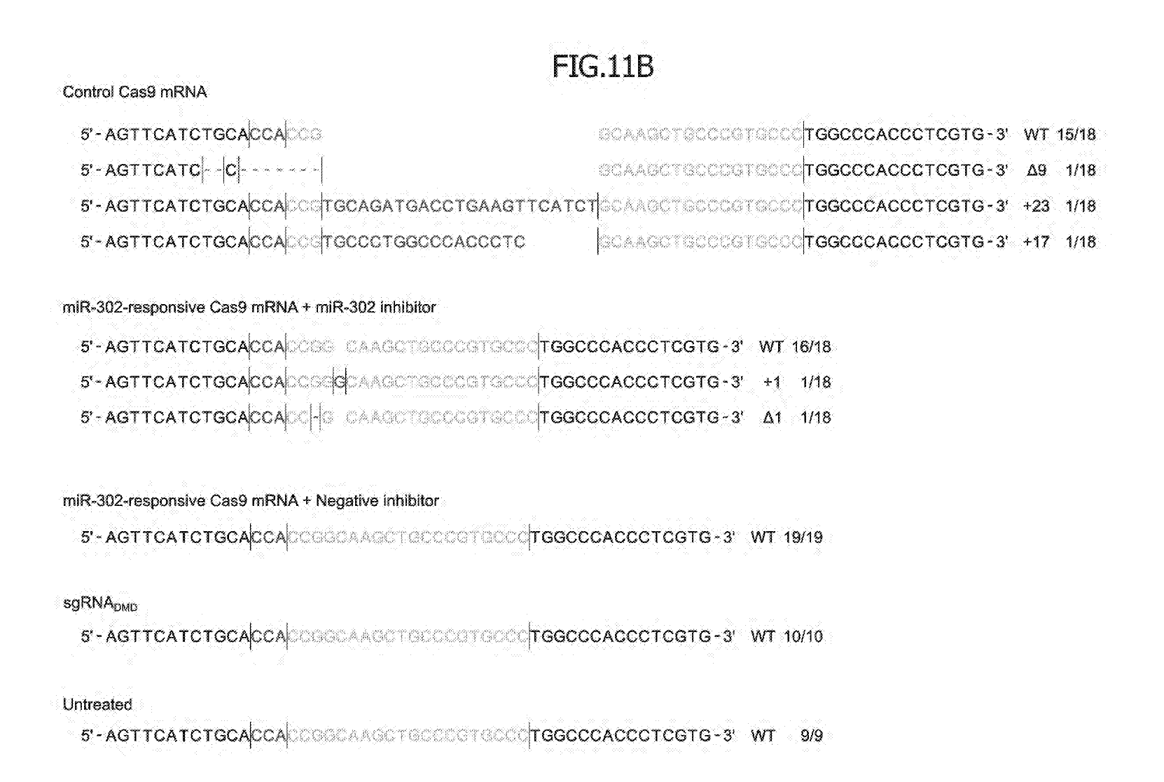

Next, as a more specific example, the method of the present invention when using a Cas9 protein as a nuclease will be described. According to one embodiment, the present invention comprises a step of co-introducing into a cell a trigger protein-responsive mRNA encoding a Cas9 protein; an miRNA-responsive mRNA encoding the trigger protein; and an sgRNA comprising a guide sequence specifically recognized by a target sequence for the Cas9 protein. FIG. 11(a) is an illustration schematically showing the action when introducing into a cell L7Ae-responsive mRNA encoding a Cas9 protein and an miRNA-responsive mRNA encoding L7Ae. In the presence of a given miRNA, the nuclease activity is enhanced by the mechanism shown in the preceding paragraph, and genome editing of the sgRNA target gene and specifically the cleavage activity of the sgRNA target DNA can be enhanced.

### EXAMPLES

Hereinafter, the present invention will be described in more detail by way of the following examples, but will not be limited by the examples.

Construction of Template DNA for IVT (In Vitro Transcription)

The 5′ UTR template (containing no miRNA target sequence) and the 3′ UTR template were PCR amplified using the corresponding primers and KOD-Plus-Neo (KOD-401, Toyobo Co., Ltd.) in the following cycles (94° C. for 2 min followed by 13 cycles of 98° C. for 10 sec and 68° C. for 10 sec and storage at 4° C.). The gene encoding a Cas9 protein was PCR amplified from the template plasmid (pHL-EFla-SphcCas94C-A) using the corresponding primer and KOD-Plus-Neo (KOD-401, Toyobo Co., Ltd.) in the following cycles (94° C. for 2 min followed by 20 cycles of 98° C. for 10 sec and 68° C. for 140 sec and storage at 4° C.).

A full DNA template as a template for IVT was constructed by PCR using the PCR products constructed above and the primers each of which corresponded to each of PCR products, respectively (using an oligo DNA instead of the 5′ UTR when inserting an miRNA target sequence). The template for a control Cas9 mRNA was constructed by PCR under the conditions of 94° C. for 2 min followed by 20 cycles of 98° C. for 10 sec and 68° C. for 140 sec and storage at 4° C.; and the template for a miRNA-responsive Cas9 mRNA was constructed by PCR under the conditions of 94° C. for 2 min followed by 20 cycles of 98° C. for 10 sec, 60° C. for 30 sec and 68° C. for 140 sec and storage at 4° C. The template for an sgRNA was constructed using two primers by PCR under the conditions of 98° C. for 30 sec followed by 20 cycles of 98° C. for 10 sec, 57° C. for 30 sec and 68° C. for 6 sec, followed by reaction at 72° C. for 10 min and then storage at 4° C.

All of the PCR products were purified by a MinElute PCR purification kit (QIAGEN), provided that those constructed using plasmids in the PCR reaction were treated with a restriction enzyme, Dpn I before purification. The sequences of the corresponding primers and oligonucleotides are shown in Tables 9A and Table 9B.

TABLE 9A

| Primer/ | | |
|---|---|---|
| Oligo DNA | | Sequence |
| name | Sequence (5′ → 3′) | ID No. |
| TAP_T7_G3C | CAGTGAATTGTAATACGACTCACTATA | 12 |
| fwd primer | GGGC | |
| IVT_5prime_ | CAGTGAATTGTAATACGACTCACTATA | 13 |
| UTR primer | GGGCGAATTAAGAGAGAAAAGAAGAGT | |
| | AAGAAGAAATATAAGACACCGGTCGCC | |
| | ACCATG | |
| Rev5UTR | CATGGTGGCGACCGGTGTCTTATATTT | 14 |
| primer | CTTCTTACTC | |
| SphcCas | CACCGGTCGCCACCATGGATAAGAAAT | 15 |
| 9 ORF fwd | ACAGCATTGGAC | |
| primer | | |
| SphcCas | GCCCCGCAGAAGGTCTAGACTATCACA | 16 |
| 9 ORF rev | CCTTCCTCTTCTTCTTGG | |
| primer | | |
| IVT_3prime_ | TCTAGACCTTCTGCGGGGCTTGCCTTC | 17 |
| UTR primer | TGGCCATGCCCTTCTTCTCTCCCTTGC | |
| | ACCTGTACCTCTTGGTCTTTGAATAAA | |
| | GCCTGAGTAGG | |
| Fwd3UTR | TCTAGACCTTCTGCGGGGC | 18 |
| primer | | |
| Rev3UTR | TTTTTTTTTTTTTTTTTTTTCCTACTC | 19 |
| 2T20 | AGGCTTTATTCAAAGACCAAG | |
| GCT7pro_ | GCTAATACGACTCACTATAGGTTCCTT | 20 |
| 5UTR2 | AATCGCGGATCC | |
| 5UTRtemp_ | CGACTCACTATAGGTTCCGCGATCGCG | 21 |
| T302a-5p | GATCCAGCAAGTACATCCACGTTTAAG | |
| | TAGATCCACCGGTCGCCACCATG | |
| 5UTRtemp_ | CGACTCACTATAGGTTCCGCGATCGCG | 22 |
| T21-5P | GATCCTCAACATCAGTCTGATAAGCTA | |
| | AGATCACACCGGTCGCCACCATG | |
| 5UTRtemp_ | CGACTCACTATAGGTTCCGCGATCGCG | 23 |
| 4x302a-5p | GATCCagcaagtacatccacgtttaag | |
| | tagcaagtacatccacgtttaagtagc | |
| | aagtacatccacgtttaagtagcaagt | |
| | acatccacgtttaagtAGATCACACCG | |
| | GTCGCCACCATG | |
| 3UTR120A | TTTTTTTTTTTTTTTTTTTTTTTTTTT | 24 |
| | TTTTTTTTTTTTTTTTTTTTTTTTTTT | |
| | TTTTTTTTTTTTTTTTTTTTTTTTTTT | |
| | TTTTTTTTTTTTTTTTTTTTTTTTTTT | |
| | TTTTTTTTTTTTCCTACTCAGGCTTTA | |
| | TTCA | |
| T7-sgRNA | GAAATTAATACGACTCACTATAGGTAT | 25 |
| fwd primer | CTTACAGGAACTCCGTTTTAGAGCTAG | |
| (DMD) | AAATAGCAAG | |
| T7-sgRNA | GAAATTAATACGACTCACTATAGGGGG | 26 |
| fwd primer | CACGGGCAGCTTGCCGGGTTTTAGAGC | |
| (EGFP) | TAGAAATAGCAAG | |
| T8-sgRNA | GAAATTAATACGACTCACTATAGGGCA | 27 |
| fwd primer | CGGGCAGCTTGCCGGGTTTTAGAGCTA | |
| (EGFP ΔGG) | GAAATAGCAAG | |
| T7-sgRNA | GAAATTAATACGACTCACTATAGGGCC | 28 |
| fwd primer | TGTAATCCCAGCACTTTGTTTTAGAGC | |
| (Alu1) | TAGAAATAGCAAG | |
| sgRNA + 85 | AAAGCACCGACTCGGTGCCACTTTTTC | 29 |
| rev primer | AAGTTGATAACGGACTAGCCTTATTTT | |
| | AACTTGCTATTTCTAGCTCTAAAAC | |

TABLE 9B

| | | |
|---|---|---|
| T7E1_EGFP | CAGCCATTGCCTTTTATGG | 30 |
| Fwd_L | | |
| T7E1_EGFP | GTGTTCTGCTGGTAGTGGTCGGCGAG | 31 |
| Rev_Univ.2 | | |
| T7E1_EGFP | TGAGCAAGGGCGAGGAGCTGTTCAC | 32 |
| Fwd_ORF | | |

Construction and Purification of Cas9 mRNA and sgRNA

A Cas9 mRNA was constructed with a MegaScript kit (Ambion, Inc.). At this time, modified bases, pseudouridine-5′-triphosphate and 5-methylcytidine-5′-triphosphate (TriLink Bio Technologies, Inc.) were added instead of UTP and CTP, respectively to suppress the immune reaction. GTP was diluted 5-fold with Anti Reverse Cap Analog (TriLink Bio Technologies, Inc.). An sgRNA was constructed using natural bases (ATP, GTP, CTP and UTP) with an MEGAshortscript kit (Ambion, Inc.). Each of the reaction mixtures was incubated at 37° C. for 6 hours followed by addition of TURBO DNase (Ambion, Inc.) thereto and further incubation at 37° C. for 30 minutes. The resulting mRNA was purified with a FavorPrep Blood/Cultured Cells total RNA extraction column (Favorgen Biotech Corp.) and incubated at 37° C. for 30 minutes using Antarctic phosphatase (New England Biolabs). Thereafter, further purification was performed with an RNeasy MinElute Cleanup Kit (QIAGEN). The sgRNA was purified, and further excised and purified with urea-PAGE (10%). The sequences of the coding region and the 5′ UTR and the 3′ UTR of the Cas9 mRNA are shown in Table 10A and Table 10B below.

TABLE 10A

| RNA | | Sequence |
|---|---|---|
| name | Sequence (5′ → 3′) | ID No. |
| Cas9 | AUGGAUAAGAAAUACAGCAUUGGACUGGACAUU | 33 |
| coding | GGGACAAACUCCGUGGGAUGGGCCGUGAUUACA | |
| region | GACGAAUACAAAGUGCCUUCAAAGAAGUUCAAG | |
| | GUGCUGGGCAACACCGAUAGACACAGCAUCAAG | |
| | AAAAAUCUGAUUGGAGCCCUGCUGUUCGACUCC | |
| | GGCGAGACAGCUGAAGCAACUCGGCUGAAAAGA | |
| | ACUGCUCGGAGAAGGUAUACCCGCCGAAAGAAU | |
| | AGGAUCUGCUACCUGCAGGAGAUUUUCAGCAAC | |
| | GAAAUGGCCAAGGUGGACGAUAGUUUCUUUCAC | |
| | CGCCUGGAGGAAUCAUUCCUGGUCGAGGAAGAU | |
| | AAGAAACACGAGCGGCAUCCCAUCUUUGGCAAC | |
| | AUUGUGGACGAGGUCGCUUAUCACGAAAAGUAC | |
| | CCUACCAUCUAUCAUCUGAGGAAGAAACUGGUG | |
| | GACUCCACAGAUAAAGCAGACCUGCGCCUGAUC | |
| | UAUCUGGCCCUGGCUCACAUGAUUAAGUUCCGG | |
| | GGCCAUUUUCUGAUCGAGGGGGAUCUGAACCCA | |
| | GACAAUUCUGAUGUGGACAAGCUGUUCAUCCAG | |
| | CUGGUCCAGACAUACAAUCAGCUGUUUGAGGAA | |
| | AACCCCAUUAAUGCAUCUGGCGUGGACGCAAAA | |
| | GCCAUCCUGAGUGCCAGACUGUCUAAGAGUCGG | |
| | AGACUGGAGAACCUGAUCGCUCAGCUGCCAGGG | |
| | GAAAAGAAAAACGGCCUGUUUGGGAAUCUGAUU | |
| | GCACUGUCACUGGGACUGACUCCCAACUUCAAG | |
| | AGCAAUUUUGAUCUGGCCGAGGACGCUAAACUG | |
| | CAGCUGUCCAAGGACACCUAUGACGAUGACCUG | |
| | GAUAACCUGCUGGCUCAGAUCGGGGAUCAGUAC | |
| | GCAGACCUGUUCCUGGCCGCUAAGAAUCUGUCU | |
| | GACGCCAUCCUGCUGAGUGAUAUUCUGCGCGUG | |
| | AACACCGAGAUUACAAAAGCCCCCCUGUCAGCU | |
| | AGCAUGAUCAAGAGAUAUGACGAGCACCAUCAG | |
| | GAUCUGACCCUGCUGAAGGCUCUGGUGAGGCAG | |
| | CAGCUGCCUGAGAAGUACAAGGAAAUCUUCUUU | |
| | GAUCAGUCUAAGAACGGAUACGCCGGCUAUAUU | |
| | GACGGCGGGGCUAGUCAGGAGGAGUUCUACAAG | |
| | UUUAUCAAACCCAUUCUGGAGAAGAUGGAUGGC | |
| | ACAGAGGAACUGCUGGUGAAACUGAAUCGGGAA | |
| | GACCUGCUGAGGAAGCAGCGCACUUUUGAUAAC | |
| | GGAAGCAUCCCUCACCAGAUUCAUCUGGGAGAG | |
| | CUGCACGCAAUCCUGAGGCGCCAGGAAGACUUC | |
| | UACCCAUUUCUGAAGGAUAACAGGGAGAAGAUC | |
| | GAAAAAAUUCUGACAUUCCGCAUCCCCUACUAU | |
| | GUGGGCCCUCUGGCAAGAGGCAACAGCCGGUUU | |
| | GCCUGGAUGACUCGCAAAUCUGAGGAAACAAUC | |
| | ACUCCCUGGAACUUCGAGGAAGUGGUCGAUAAG | |
| | GGCGCUUCCGCACAGUCUUUCAUUGAGCGGAUG | |
| | ACAAACUUCGACAAGAACCUGCCAAACGAAAAA | |
| | GUGCUGCCCAAGCACUCUCUGCUGUACGAGUAU | |
| | UUCACAGUCUAUAACGAACUGACUAAGGUGAAA | |
| | UACGUCACCGAGGGGAUGAGAAAGCCUGCCUUC | |
| | CUGAGUGGAGAACAGAAGAAAGCUAUCGUGGAC | |
| | CUGCUGUUUAAAACCAAUAGGAAGGUGACAGUC | |
| | AAGCAGCUGAAAGAGGACUAUUUCAAGAAAAUU | |
| | GAAUGUUUCGAUUCUGUGGAGAUCAGUGGCGUC | |
| | GAAGACAGGUUUAACGCCUCCCUGGGGACCUAC | |
| | CACGAUCUGCUGAAGAUCAUUAAGGAUAAAGAC | |
| | UUCCUGGACAACGAGGAAAAUGAGGAUAUCCUG | |
| | GAAGACAUUGUGCUGACCCUGACACUGUUUGAG | |
| | GAUAGGGAAAUGAUCGAGGAACGCCUGAAGACC | |
| | UAUGCCCAUCUGUUCGAUGACAAAGUGAUGAAA | |
| | CAGCUGAAGCGACGGAGAUACACAGGAUGGGGC | |
| | CGACUGUCUCGGAAGCUGAUCAAUGGGAUUCGC | |
| | GACAAACAGAGUGGAAAGACCAUCCUGGACUUU | |
| | CUGAAAUCAGAUGGCUUCGCCAACCGGAACUUC | |
| | AUGCAGCUGAUUCACGAUGACAGCCUGACAUUC | |
| | AAAGAGGAUAUCCAGAAGGCACAGGUGUCCGGG | |
| | CAGGGAGACUCUCUGCACGAGCAUAUCGCAAAC | |
| | CUGGCCGGCAGCCCUGCCAUCAAGAAAGGGAUU | |
| | CUGCAGACCGUGAAGGUGGUGGACGAGCUGGUG | |
| | AAAGUCAUGGGAAGACAUAAGCCAGAAAACAUC | |
| | GUGAUUGAGAUGGCCAGGGAAAAUCAGACCACA | |
| | CAGAAAGGCCAGAAGAACUCAAGGGAGCGCAUG | |
| | AAAAGAAUCGAGGAAGGAAUUAAGGAACUGGGC | |
| | AGCCAGAUCCUGAAAGAGCACCCCGUGGAAAAC | |
| | ACACAGCUGCAGAAUGAGAAGCUGUAUCUGUAC | |
| | UAUCUGCAGAAUGGACGCGAUAUGUACGUGGAC | |
| | CAGGAGCUGGAUAUUAACCGACUGUCCGAUUAC | |
| | GACGUGGAUCAUAUCGUCCCACAGUCAUUCCUG | |
| | AAAGAUGACAGCAUUGACAAUAAGGUGCUGACC | |
| | CGCUCUGACAAAAACCGAGGCAAGAGUGAUAAU | |
| | GUCCCCUCAGAGGAAGUGGUCAAGAAAAUGAAG | |
| | AACUACUGGAGGCAGCUGCUGAAUGCCAAACUG | |
| | AUCACACAGCGAAAGUUUGAUAACCUGACUAAA | |
| | GCUGAGCGGGGAGGCCUGAGUGAACUGGACAAA | |
| | GCAGGCUUCAUUAAGCGACAGCUGGUGGAGACA | |
| | CGGCAGAUCACAAAGCACGUCGCCCAGAUUCUG | |
| | GAUUCAAGAAUGAACACUAAGUACGAUGAGAAU | |
| | GACAAACUGAUCAGAGAAGUGAA | |

TABLE 10B

| | | |
|---|---|---|
| | GGUCAUUACCCUGAAGUCAAAACUGGUGAG | |
| | CGACUUUCGGAAAGAUUUCCAGUUUUAUAA | |
| | GGUCAGAGAGAUCAACAACUACCACCAUGC | |
| | UCAUGACGCAUACCUGAACGCAGUGGUCGG | |
| | CACAGCCCUGAUUAAGAAAUACCCUAAACU | |
| | GGAGUCCGAGUUCGUGUACGGGGACUAUAA | |
| | GGUGUACGAUGUCAGAAAAAUGAUCGCCAA | |
| | GUCUGAGCAGGAAAUUGGCAAAGCCACUGC | |
| | UAAGUAUUUCUUUUACAGUAACAUCAUGAA | |
| | UUUCUUUAAGACUGAGAUCACCCUGGCAAA | |
| | UGGGGAAAUCCGAAAGCGGCCACUGAUUGA | |
| | GACUAACGGCGAGACAGGAGAAAUCGUGUG | |
| | GGACAAAGGAAGAGAUUUUGCUACCGUGAG | |
| | GAAGGUCCUGAGCAUGCCCCAAGUGAAUAU | |
| | UGUCAAGAAAACAGAGGUGCAGACUGGGGG | |
| | AUUCAGUAAGGAAUCAAUUCUGCCUAAACG | |
| | CAACUCCGAUAAGCUGAUCGCCCGAAAGAA | |
| | AGACUGGGACCCCAAGAAGUAUGGCGGGUU | |
| | CGACUCCCCAACUGUGGCUUACUCUGUCCU | |
| | GGUGGUCGCAAAGGUGGAGAAGGGAAAAAG | |
| | CAAGAAACUGAAAUCCGUCAAGGAACUGCU | |
| | GGGCAUCACCAUUAUGGAGCGCAGCUCCUU | |
| | CGAAAAGAAUCCUAUCGAUUUUCUGGAGGC | |
| | CAAAGGCUAUAAGGAAGUGAAGAAAGACCU | |
| | GAUCAUCAAGCUGCCAAAGUACUCACUGUU | |
| | UGAGCUGGAAAACGGGAGAAAGAGGAUGCU | |
| | GGCAAGCGCCGGGGAGCUGCAGAAAGGAAA | |
| | UGAACUGGCCCUGCCCUCCAAGUACGUGAA | |
| | CUUCCUGUAUCUGGCUAGCCACUACGAGAA | |
| | GCUGAAAGGGUCCCCUGAGGAUAACGAACA | |
| | GAAACAGCUGUUUGUGGAGCAGCACAAGCA | |
| | UUAUCUGGACGAGAUCAUUGAACAGAUUAG | |
| | CGAGUUCUCCAAAAGAGUGAUCCUGGCUGA | |
| | CGCAAAUCUGGAUAAGGUCCUGAGCGCAUA | |
| | CAACAAACACCGGGAUAAGCCAAUCAGAGA | |
| | GCAGGCCGAAAAUAUCAUUCAUCUGUUCAC | |
| | UCUGACCAACCUGGGAGCCCCCGCAGCCUU | |
| | CAAGUAUUUUGACACUACCAUCGAUCGCAA | |
| | ACGAUACACAAGCACUAAGGAGGUGCUGGA | |
| | CGCUACCCUGAUUCAUCAGAGCAUUACUGG | |
| | CCUGUAUGAAACAAGGAUUGACCUGUCUCA | |
| | GCUGGGCGGCGACUCCGGAGCUGACCCCAA | |
| | GAAGAAGAGGAAGGUGUGA | |
| 3′UTR | UAGUCUAGACCUUCUGCGGGGCUUGCCUUC | 34 |
| | UGGCCAUGCCCUUCUUCUCUCCCUUGCACC | |
| | UGUACCUCUUGGUCUUUGAAUAAAGCCUGA | |
| | GUAGGAAAAAAAAAAAAAAAAAAAAAAAAA | |
| | AAAAAAAAAAAAAAAAAAAAAAAAAAAAAA | |
| | AAAAAAAAAAAAAAAAAAAAAAAAAAAAAA | |
| | AAAAAAAAAAAAAAAAAAAAAAAAAAAAAA | |
| | AAAAA | |
| 5′UTR_ | GGGCGAAUUAAGAGAGAAAAGAAGAGUAAG | 35 |
| Control | AAGAAAUAUAAGACACCGGUCGCCACC | |
| Cas9 mRNA | | |

Cultured Cells

iPS_GFP cells (AAVS1-CAG::GFP iPS cells) were provided by Woltjen Lab (CiRA, Kyoto University, Japan). The iPS_GFP cells were cultured using StemFit (Ajinomoto Co., Inc.) in a plate coated with laminin {laminin-511 E8 (iMatrix-511, Nippi, Incorporated)}. HeLa_GFP cells were cultured in a medium having DMEM High Glucose (Nacalai Tesque, Inc.) supplemented with FBS (Japan Bio System, final concentration: 10%) and hygromycin B (50 mg/mL). Normal HeLa cells were cultured in a medium having DMEM High Glucose (Nacalai Tesque, Inc.) supplemented with FBS (Japan Bio System, final concentration: 10%). All cells were cultured under the conditions of 37° C. and 5% CO₂.

Differentiation Induction (Differentiation Induction of iPS_GFP to mDA_GFP)

iPS_GFP cells were reseeded (5×10⁶ cells/well) using a differentiation induction medium in a laminin-coated 6-well plate on Day 0, and cultured according to a modified protocol of Morizana et al., Neural Development: Methods and Protocols, Methods in Molecular Biology, vol. 1018, DOI 10.1007/978-1-62703-444-9_2. Thereafter, the medium was replaced daily. The cells were used for each experiment after 14 days. The composition of each of the differentiation induction mediums is shown in Table 11 below.

TABLE 11

| Day0 | 10 mL | 40 mL |
|---|---|---|
| 8GMK (mL) | 10 | 40 |
| LDN (μL) | 1 | 4 |
| A-83-01 (μL) | 1 | 4 |
| Y27632 (μL) | 20 | 80 |
| Day1-2 | 10 mL | 40 mL |
| 8GMK (mL) | 10 | 40 |
| LDN (μL) | 1 | 4 |
| A-83-01 (μL) | 1 | 4 |
| Purmorph (μL) | 2 | 8 |
| FGF8 (μL) | 10 | 40 |
| Day3-6 | 10 mL | 40 mL |
| 8GMK (mL) | 10 | 40 |
| LDN (μL) | 1 | 4 |
| A-83-01 (μL) | 1 | 4 |
| Purmorph (μL) | 2 | 8 |
| FGF8 (μL) | 10 | 40 |
| CHIR (μL) | 10 | 40 |
| Day7-11 | 10 mL | 40 mL |
| 8GMK (mL) | 10 | 40 |
| LDN (μL) | 1 | 4 |
| CHIR (μL) | 10 | 40 |
| Day12- | 10 mL | 40 mL |
| NB B27 (mL) | 10 | 40 |
| GDNF (μL) | 10 | 40 |
| BDNF | 2 | 8 |
| AA | 10 | 40 |
| dbcAMP | 10 | 40 |
| | Stock conc. | |
| LDN | 1 | mM |
| A-83-01 | 5 | mM |
| Purmorph | 10 | mM |
| FGF8 | 100 | μg/mL |
| CHIR | 3 | mM |
| BDNF | 25 | μg/mL |
| GDNF | 10 | μg/mL |
| AA | 200 | mM |
| dbcAMP | 400 | mM |
| Y27632 | 5 | mM |

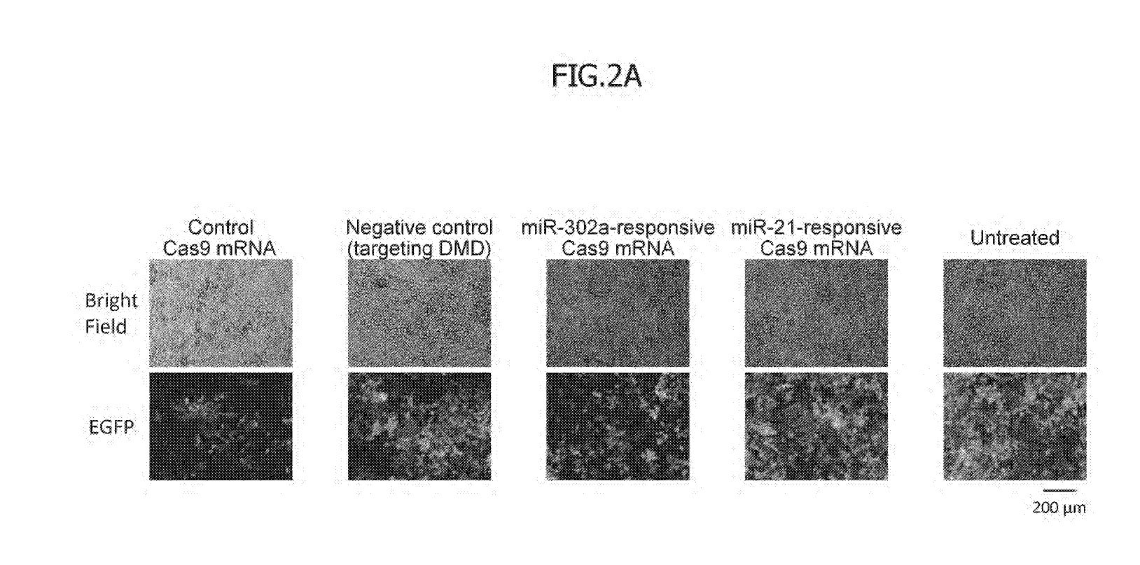

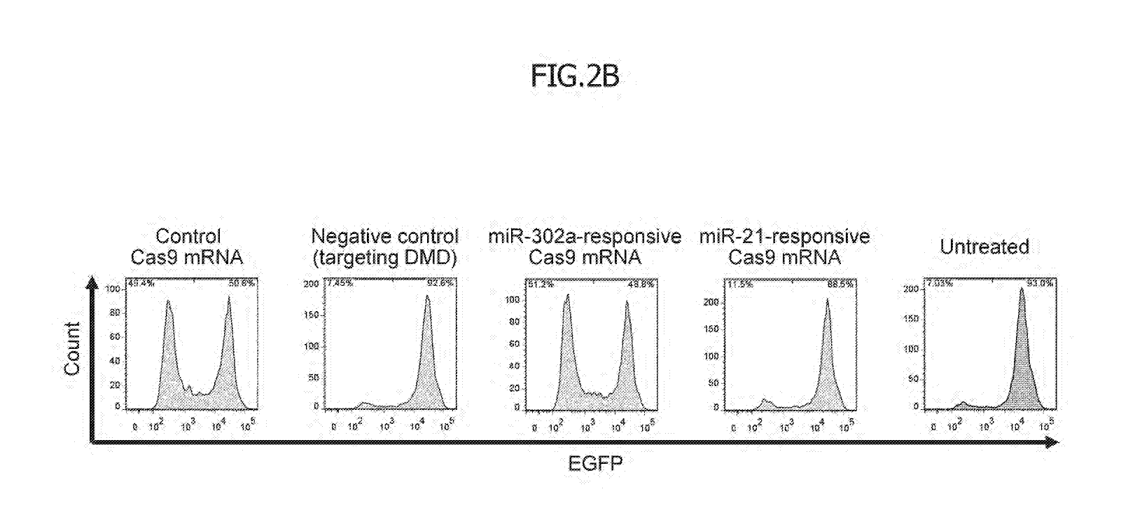

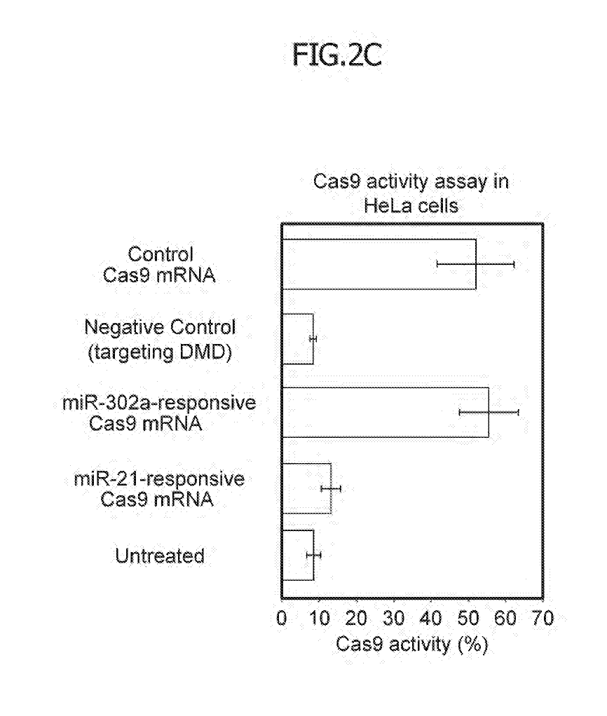

Transfection Each of normal HeLa cells and HeLa_GFP cells were seeded in a 24-well plate. Each of iPS_GFP cells and mDA_GFP cells were seeded in a laminin-coated 24-well plate (cell number: 5×10⁴ cells/well). Each transfection was performed using a Stemfect RNA transfection kit (Stemgent) according to the protocol (See the respective experimental sections for the transgene amounts). The medium was replaced 4 hours after transfection (except for mDA_GFP cells). Cell killing was analyzed 48 hours after transfection, and the T7E1 assay, EGFP activity assay and co-culture were analyzed 72 hours after transfection. Prior to each analysis, cells were photographed by a IX 81 microscope (Olympus Corporation) (FIG. 2).

T7E1 Assay

A Cas9 mRNA in the amount of 100 ng and an sgRNA in the amount of 300 ng (iPS_GFP) or 100 ng (mDA_GFP) were used. The transfected cells were washed with PBS and then treated with 200 μL Accumax (Funakoshi Co., Ltd.) for 10 minutes under the conditions of 37° C. and 5% CO₂. The cells were collected in a 1.5 mL tube and centrifuged (at 1000 rpm at room temperature for 5 minutes). After discarding a supernatant, precipitated cells were washed with PBS and centrifuged under the same conditions as above. 500 mL of proteinase K (×100; final concentration: 1×) was added to a lysis buffer (1 M Tris-HCl (pH 7.6) [final concentration: 0.05 M], 0.5 M EDTA [final concentration: 0.02 M], 5 M NaCl [0.1 M], 10% SDS [final concentration: 1%], D2W), with which the cells were treated at 55° C. for 3 hours or more. Thereafter, genomic DNAs were extracted using PCI. Each of the target sequences was amplified from each of the extracted genomic DNAs by Nested PCR. The first PCR was performed under the conditions of 94° C. for 2 min followed by 20 cycles of 98° C. for 10 sec, 60° C. for 30 sec and 68° C. for 30 sec, followed by reaction at 72° C. for 3 min and then storage at 4° C. The second PCR was performed under the conditions of 94° C. for 2 min followed by 35 cycles 98° C. for 10 sec, 60° C. for 30 sec and 68° C. for 15 sec, followed by reaction at 72° C. for 3 min and then storage at 4° C. PCR products were purified by a MinElute PCR purification kit (QIAGEN). After purification, the PCR products were subjected to denaturation and reassociation under the conditions of 95° C. for 5 min followed by cooling at 2° C./sec from 95° C. to 85° C. and at 0.1° C./sec from 85° C. to 25° C. and then storage at 4° C. After the reaction, they were treated with a restriction enzyme, T7 Endonuclease I (at 37° C. for 15 min). After 15 minutes, 0.5 M EDTA was added thereto to terminate the reaction, 5% polyacrylamide gel electrophoresis was performed, staining with a SYBR GREEN mixture (I+II=1:1) was performed and photographs were taken. Primers used in PCR are shown in Table 9.

Indels (Cas9 activity) were calculated by the following formula:

Indels=100×(1−sqrt(1−(b+c)/(a+b+c)))
wherein:
a represents the band strength of the PCR product not cleaved with the restriction enzyme; and
b and c represent the band strength of the PCR product cleaved with the restriction enzyme.

Cas9 Activity Assay

A Cas9 mRNA in the amount of 100 ng and an sgRNA in the amount of 300 ng (iPS_GFP or HeLa_GFP) or 100 ng (mDA_GFP) and an miRNA inhibitor (mirVana) or a negative control in the amount of 5 pmol (the miRNA inhibitor and negative control optionally used) were used. Cells were washed with PBS. Thereafter, HeLa_GFP cells were treated with 100 μL of 0.25% trypsin-EDTA under the conditions of 37° C. and 5% CO₂ for 5 minutes, and 100 μL of a medium was then added thereto. iPS_GFP cells and mDA_GFP cells were treated with 200 μL of Accumax (Funakoshi Co., Ltd.) under the conditions of 37° C. and 5% CO₂ for 10 minutes. The cells were collected in a 1.5 mL tube, respectively and treated with a CYTOX Red dead-cell stain (Thermo Fisher Scientific, Inc.) (light-shielded and left at room temperature for 15 minutes). They were measured with Aria-II (BD), Accuri (BD) and LSR (BD). The percentage of EGFP negative cells (%) was defined as the Cas9 activity (%).

Cell Killing System

A Cas9 mRNA in the amount of 10 ng and an sgRNA in the amount of 300 ng were used. Before washing with PBS, each of the mediums was collected in a 1.5 mL tube. After washing with PBS, normal HeLa cells were treated with 100 μL of 0.25% trypsin-EDTA under the conditions of 37° C. and 5% CO₂ for 5 minutes. Thereafter, 100 μL of a medium was added thereto and the cells were collected in the 1.5 mL tube and centrifuged (at 1000 rpm at room temperature for 5 minutes). After discarding a supernatant, precipitated cells were washed with PBS and centrifuged under the same conditions as above. After discarding a supernatant, the cells were stained with 53 μL of a mixture of staining reagents {annexin V, Alexa Fluor 488 conjugate (Life Technologies), Annexin-binding buffer 5× (Life Technologies), SYTOX Red dead-cell stain}. The stained cells were measured with Accuri (BD).

Co-Culture

A Cas9 mRNA in the amount of 50 ng and an sgRNA in the amount of 150 ng were used. Cells were collected in a 1.5 mL tube in the same manner as in the Cas9 activity assay in which iPS_GFP cells and HeLa_GFP cells were seeded at a ratio of 3 to 2, and then centrifuged (at 1000 rpm at room temperature for 5 minutes). After removing the supernatant, the cells were stained with Alexa Fluor® 647 Mouse anti-Human TRA-1-60 Antigen according to the protocol (using the antibody in twice the amount in the protocol), and measured with LSR.

Results

The behavior of the miRNA-responsive CRISPR/Cas9 System in HeLa cells is shown in FIG. 2. The activity of an endogenous miR-21 in HeLa cells is known to be high. FIG. 2 (a) shows fluorescence microscopic images of HeLa cells into which an miRNA-responsive mRNA was introduced. The fluorescence of GFP is weaker for the control that does not respond to the miRNA and the miRNA-302a-responsive Cas9 mRNA than those for “untreated” and the negative control for the target sequence. The negative control used was one targeting a DMD gene. On the other hand, there was no change in fluorescence in miRNA-21-responsive Cas9 mRNA. FIG. 2(a) shows the results of one experiment as a representative of three experiments. FIG. 2(b) clearly shows that the above-described fluorescence images are consistent with the quantitative results of fluorescence intensities by Aria-II analysis. FIG. 2(b) also shows the results of one experiment as a representative of three experiments. FIG. 2(c) is a graph obtained by defining as the Cas9 activity the GFP negative population in FIG. 2(b). Hereinafter, the Cas9 activity is that which was calculated by this method unless otherwise specified. From the above, it was found that among the various miRNA-responsive mRNA and the control mRNA introduced into HeLa cells, the only miR-21-responsive Cas9 mRNA was low in Cas9 activity, indicating the possibility that the miR-21-responsive Cas9 mRNA may be regulated by the miR-21.

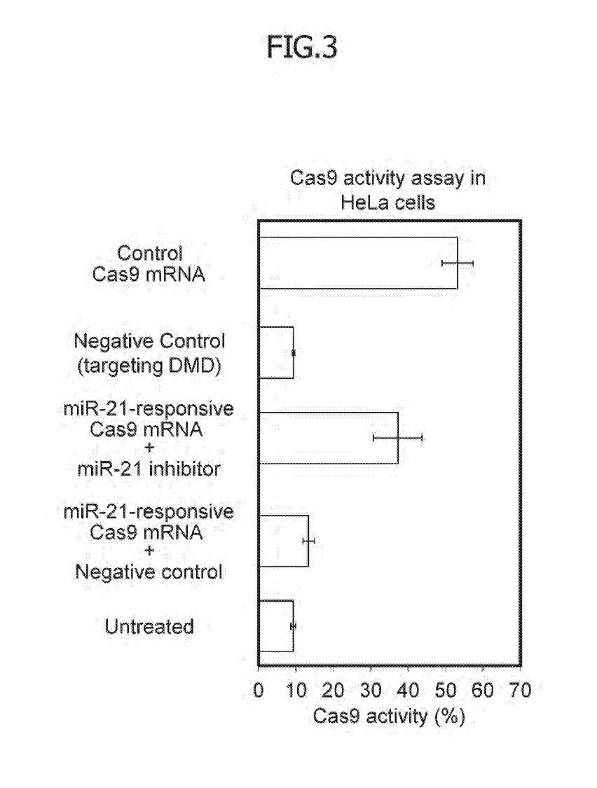

By using an miRNA-21 inhibitor, it was verified using HeLa cells whether an miRNA-21-responsive Cas9 mRNA was regulated by the endogenous miR-21. The results are shown in FIG. 3. It was clearly shown that the Cas9 activity was low in the negative control for the miRNA inhibitor (hereinafter referred to as “Negative control” in the figures), whereas the Cas9 activity was increased (rescued) when the miR-21 inhibitor was used. That is, it indicates that the miRNA-21-responsive Cas9 mRNA was regulated by the endogenous miR-21.

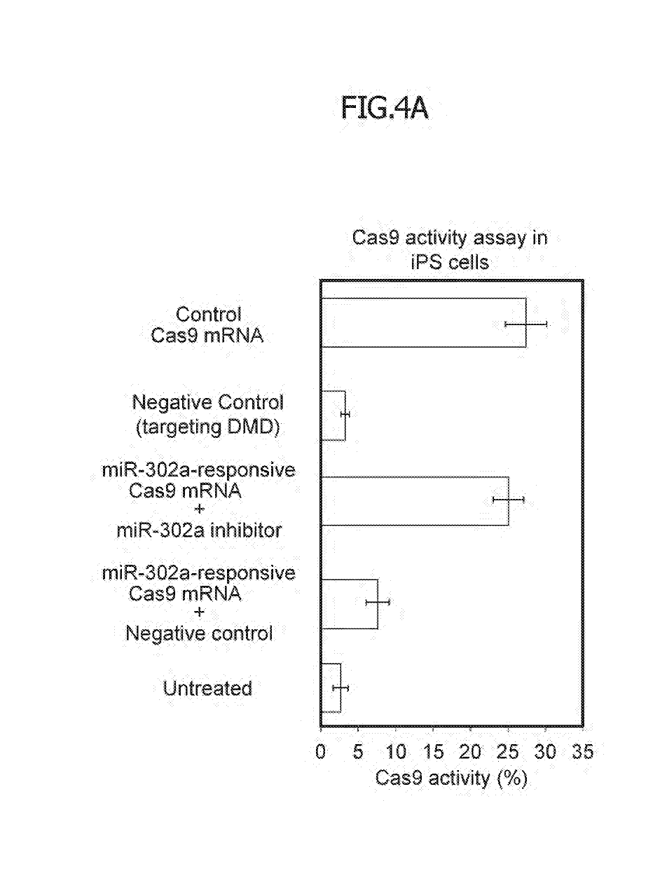

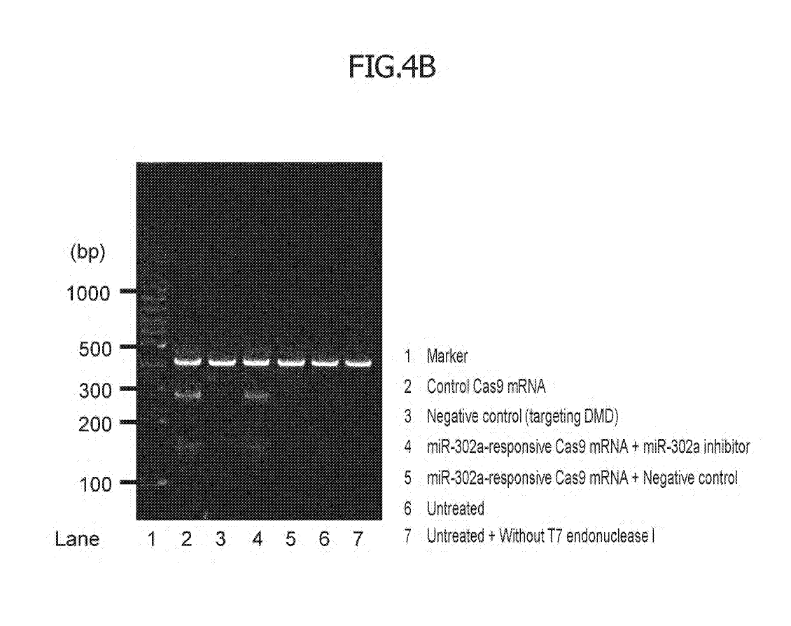

By using an miRNA-302a inhibitor, it was verified using iPS cells whether the miRNA-302a-responsive Cas9 mRNA was regulated by an endogenous miR-302a. The activity of miR-302a in iPS cells is known to be high. The results are shown in FIG. 4. The Cas9 activity was low in the negative control, whereas the Cas9 activity was rescued when the miR-302a inhibitor was used. That is, it indicates that the miRNA-302a-responsive Cas9 mRNA was regulated by the endogenous miR-302a. FIG. 4(a) shows the results of the evaluation by the Cas9 activity assay, and FIG. 4(b) shows the gel image showing the results of the evaluation by the T7E1 assay. The gel image is shown for one experiment as a representative of three experiments. FIG. 4(c) shows the indels (defined as Cas9 activities) calculated from the results of the T7E1 assay. The formula for calculation is as described in detail in the section of the T7E1 assay.

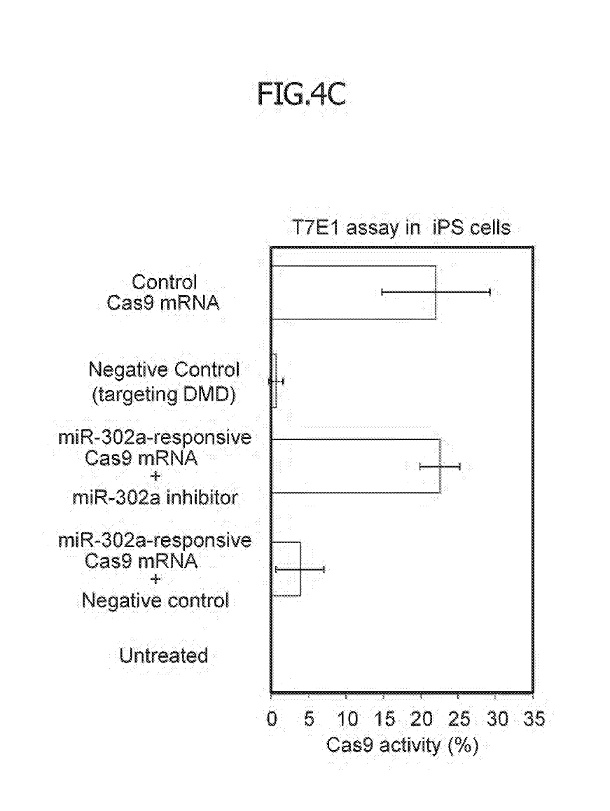

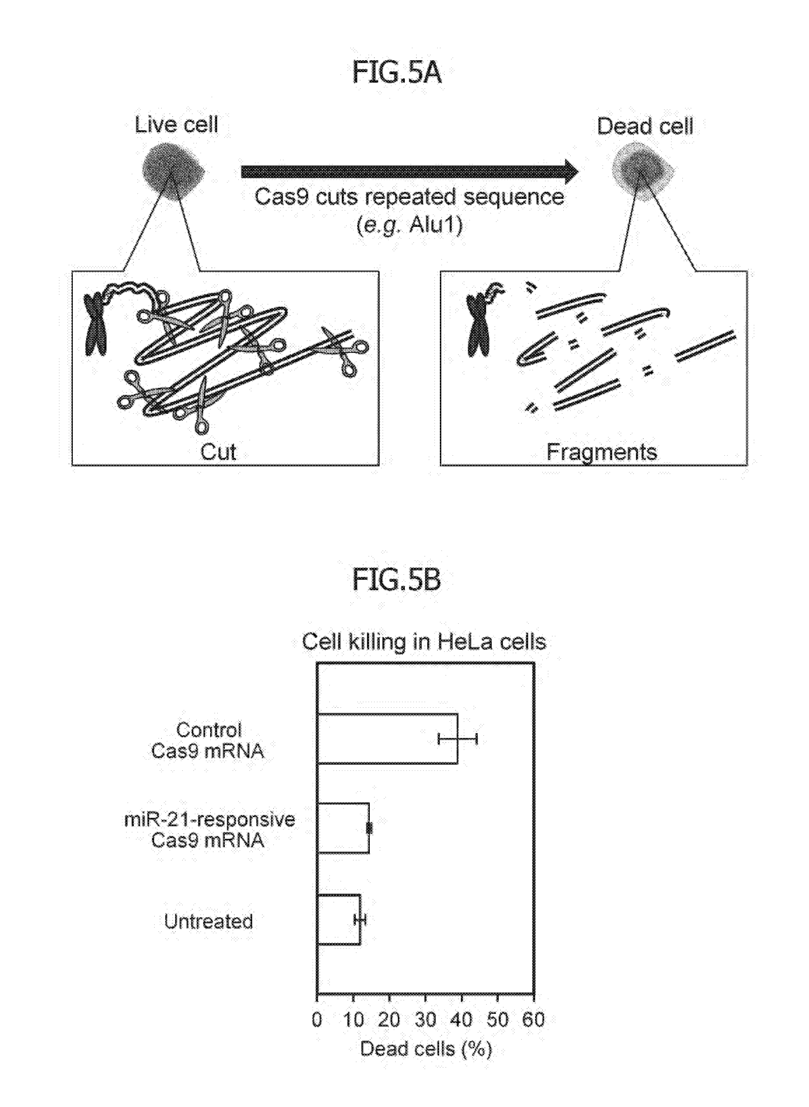

FIG. 5 is an illustration schematically showing a scheme for causing cell death with an miRNA-responsive CRISPR/Cas9 System of the present invention. FIG. 5 (a) schematically shows that Cas9 fragments a genome by making the sequence of an sgRNA a repeated sequence on the genome. The fragmentation of the genome causes cell death. In this experiment, cell death regulation was examined by using an miR-21-responsive Cas9 mRNA and targeting Alu1. The experiment was performed according to the method described in the section of Cell killing system, and it was thereby verified in HeLa cells whether it was possible to regulate cell death by using the miR-21-responsive Cas9 mRNA. The results are shown in FIG. 5(b). The miR-21-responsive Cas9 mRNA was designed to translationally repress Cas9 mRNA and decrease the Cas9 activity in cells specifically expressing miR-21. FIG. 5(b) shows that cell death was induced using the control Cas9 mRNA (the Cas9 activity is high). On the other hand, the miR-21-responsive Cas9 mRNA (the Cas9 activity is low) exhibited the rate of dead cells as low as “Untreated”. These results show that cell death was not induced by the miR-21-responsive Cas9 mRNA. This demonstrated that cell death is regulatable by using the miRNA-responsive CRISPR/Cas9 System.

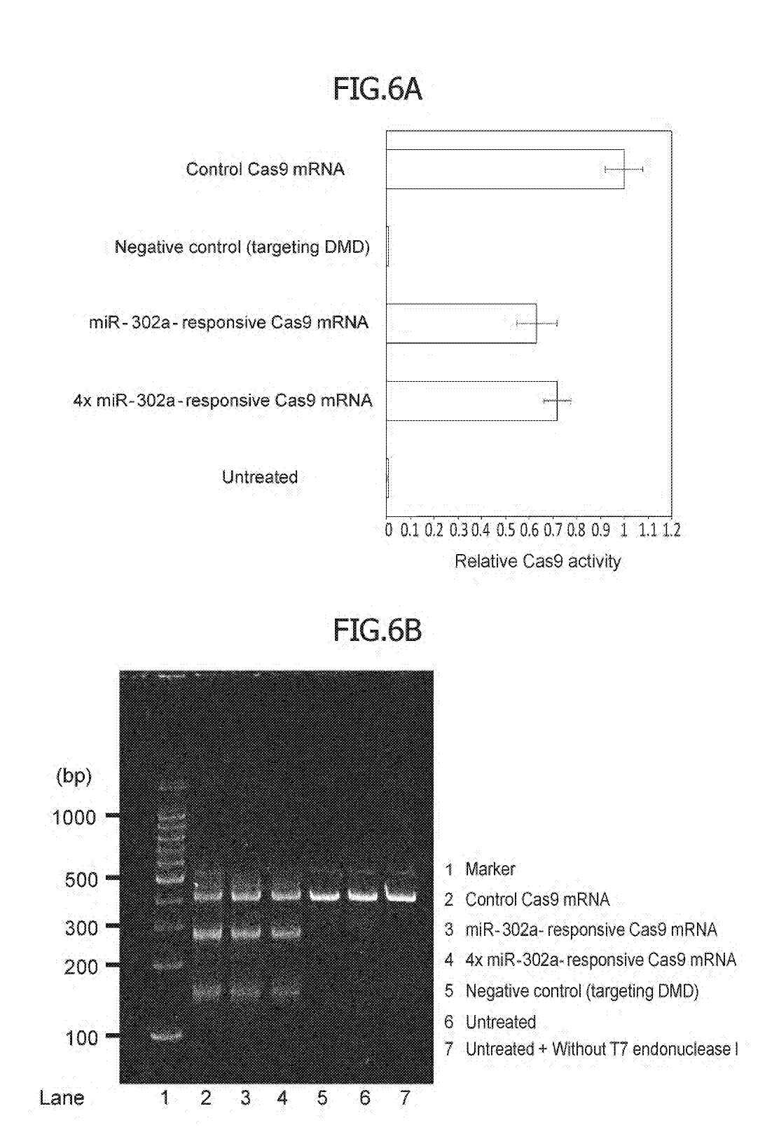

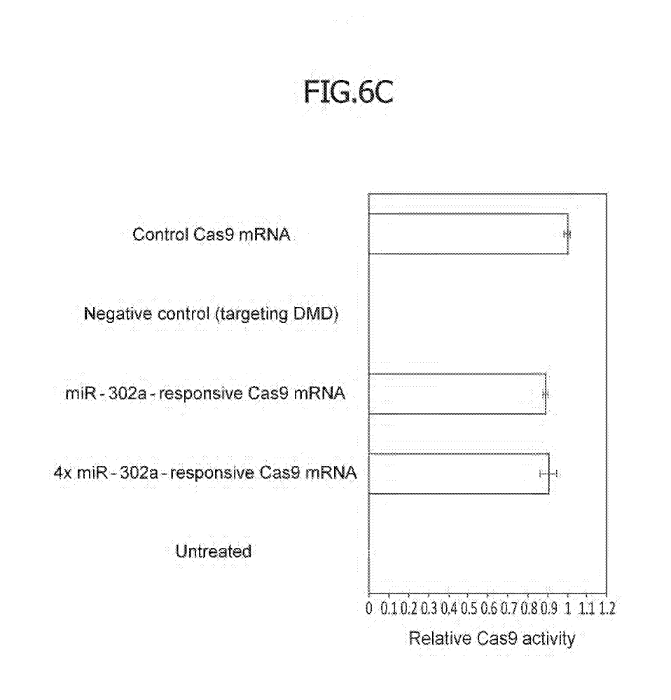

FIG. 6 shows the results of experiments in which an miR-302a-responsive Cas9 mRNA was introduced into mDA cells. An miRNA-responsive mRNA (control, 302a-responsive or 4×miR-302a-responsive) and an sgRNA (targeting GFP, or targeting DMD as a negative control) were prepared. The 4×miR-302a-responsive Cas9 mRNA has four miR-302a-5p target sequences inserted into its 5′ UTR. The mDA cells (midbrain dopaminergic neurons) were obtained by subjecting iPS cells to differentiation induction according to the method described above. Co-transfection of mDA cells was performed for 100 ng of each of the miRNA-responsive mRNA and the sgRNA, and the change in fluorescence intensity resulting from GFP knockout was evaluated according to the Cas9 activity assay. The results are shown in FIG. 6. FIG. 6(a) is a graph showing the relative Cas9 activities represented by defining the Cas9 activity as Cas9 mRNA=1. Each experiment was performed for one sample per experiment, and the experiment was performed three times independently (n=3). Each of the error bars represents a mean±a standard deviation.

Co-transfection of mDA cells was performed for 100 ng of each of the miRNA-responsive mRNA and the sgRNA prepared in the same manner as that in FIG. 6(a), and the evaluation was performed according to the T7E1 assay. FIG. 6(b) shows a representative gel image, and FIG. 6(c) shows the relative Cas9 activities represented by defining the Cas9 activity according to the T7E1 assay as Cas9 mRNA=1. Each experiment was performed for one sample per experiment, and the experiment was performed three times independently (n=3). Each of the error bars represents a mean±a standard deviation.

The results in FIG. 6 clearly show that the miRNA-responsive CRISPR/Cas9 System according to the present invention regulated the Cas9 activity in response to the cell state, i.e., miRNA. It is predicted that because differentiation of the iPS cells (in present case, mDA cells) decreased the activity of the endogenous miR-302a-5p, the activity of the miR-302a-responsive Cas9 mRNA was low in the iPS cells but high in the mDA cells (the activity is almost the same as that of the control Cas9 mRNA). Actually, when the relative Cas9 activity was calculated, the activity of miR-302a-responsive Cas9 mRNA was recovered, and was close to that of the control Cas9 mRNA.

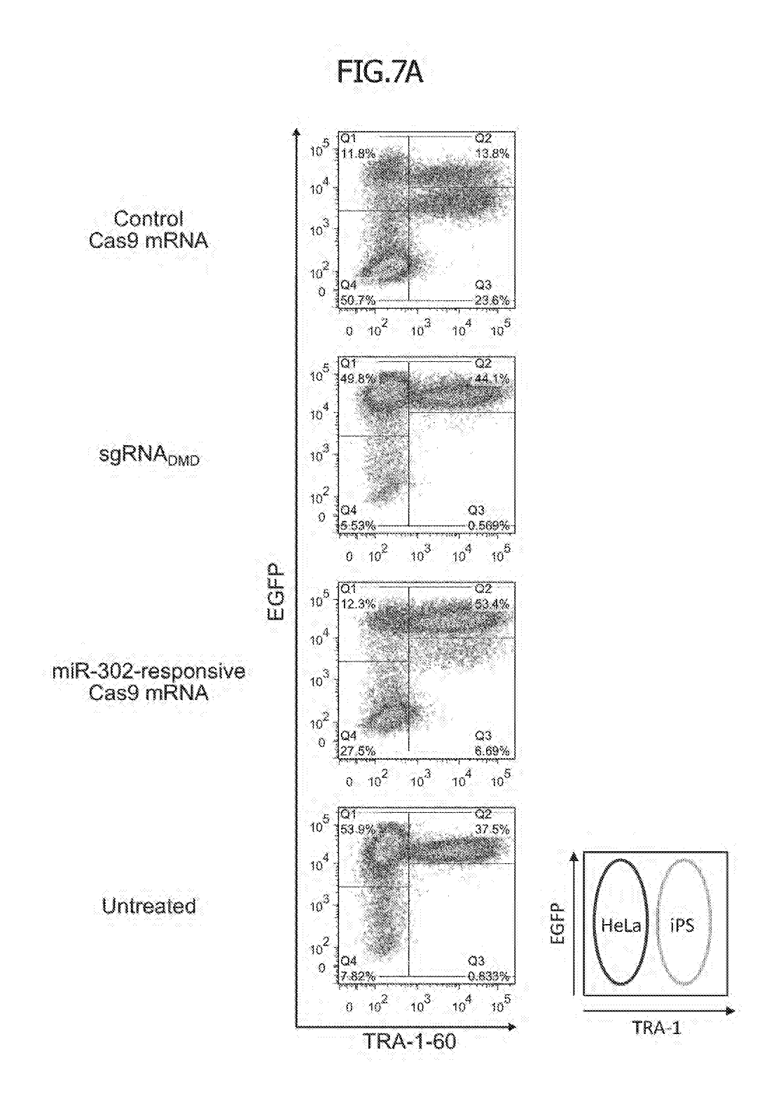

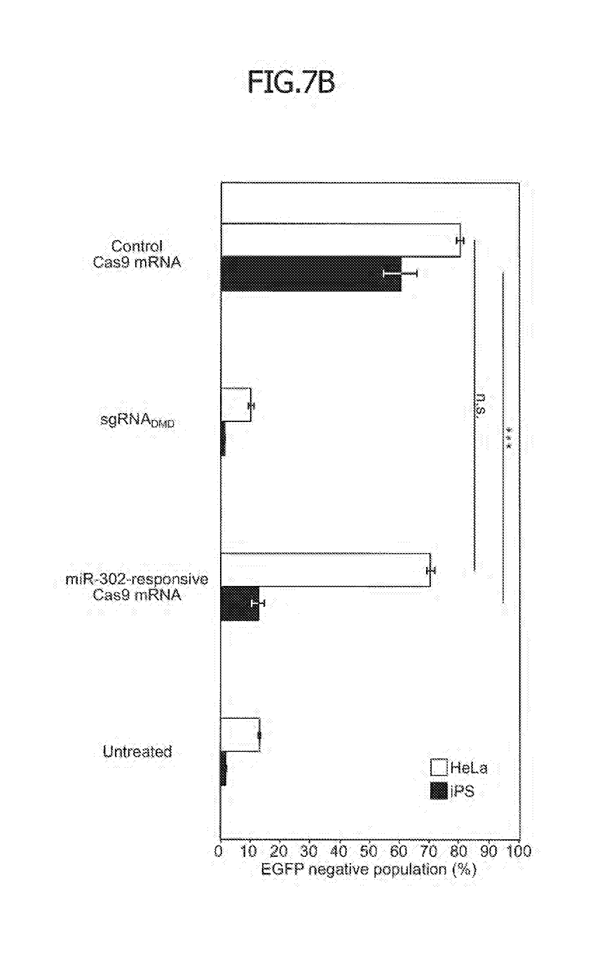

FIG. 7 shows the result of co-culture. In this experiment, each of iPS_GFP cells and HeLa_GFP cells were seeded in a 24-well plate at 5×10⁵ cells/well 17 to 24 hours before transfection. At this time, the cells were seeded at a ratio of iPS:HeLa=3:2 (cell number). The cells were transfected with 50 ng of an mRNA and 150 ng of an sgRNA using a Stemfect transfection reagent according to the protocol. The mRNAs used were a control Cas9 mRNA and a 4×miR-302a-responsive Cas9 mRNA. The sgRNAs used were an sgRNA targeting a GFP gene and an sgRNA targeting a DMD gene as a negative control. Three days after transfection, the cells were stained with Alexa Fluor® 647 Mouse anti-Human TRA-1-60 Antigen and then measured by LSR. The evaluation results of the change in fluorescence intensity by GFP knockout are shown in FIG. 7(a). Each experiment was performed for one sample per experiment and the experiment was performed three times independently (n=3). Representative dot plots are shown in the Figure. In each panel, the longitudinal axis represents the fluorescence intensity of GFP and the horizontal axis represents the fluorescence intensity of TRA-1-60. The distribution of cell populations that could be separated by TRA-1 is shown at the bottom right.

FIG. 7(b) is a graph showing the Cas9 activity calculated based on the results of FIG. 7(a) by the following formula. Each of the error bars represents a mean±a standard deviation.

Cas9 activity (%)=Q4/(Q1+Q4)×100: HeLa

Cas9 activity (%)=Q3/(Q2+Q3)×100: iPS

The results of FIG. 7 suggested that genome editing of a target cell population alone could be performed with a different cell population. Thus, this experiment succeeded in causing GFP knockout in the HeLa cells while preventing GFP knockout in the iPS cells, by utilizing the fact that the expression (activity) of miR-302a is high in the iPS cells.

Transfection

Each of normal HeLa cells and HeLa_GFP cells were seeded in a 24-well plate. Each of iPS_GFP cells and mDA_GFP cells were seeded in a laminin-coated 24-well plate (cell number: 5×10⁴ cells/well). Each transfection was performed using a Stemfect RNA transfection kit (Stemgent) according to the protocol (See the respective experimental sections for the transgene amounts). The medium was replaced 4 hours after transfection (except for mDA_GFP cells). Evaluation of the gene expression level, evaluation of the Cas9 protein expression level and evaluation of the transfection efficiency were performed 24 hours after transfection. Cell killing was analyzed 48 hours after transfection, and the T7E1 assay, EGFP activity assay, co-culture and On-system were analyzed 72 hours after transfection. Prior to each analysis, cells were photographed by a IX 81 microscope (Olympus Corporation).

On-System

An mRNA was designed as outlined in FIG. 11A. The miRNA target site used was a sequence complementary to miR-21. The mRNA was constructed by using a L7Ae protein as a trigger protein the expression of which was repressed in response to the miRNA and using a kink turn motif as a sequence that specifically binds to the L7Ae protein, in the same manner as the method described above. The sequence of the constructed mRNA (Kt-Cas9 mRNA) is shown in Table 12A and Table 12B below wherein the L7Ae binding motif is double-underlined and the Cas9 coding region is shown in italic. The sequences of mRNAs (L7Ae mRNA, miR-21-responsive L7Ae and Tag BFP mRNA) are shown in Table 13 below wherein the miR-21-5p target sequence is double-underlined and the L7Ae or Tag BFP coding region is shown in italic.

TABLE 12A

| RNA | | Sequence |
|---|---|---|
| name | Sequence (5′ → 3′) | ID No. |
| Kt-Cas9 | GGAUCCGUGAUCGGAAACGUGAGAUC | 36 |
| mRNA | CACCUCAGAUCCGCUAGGACACCCGC | |
| | AGAUCGAGAAGAAGGCGAAUUAAGAG | |
| | AGAAAAGAAGAGUAAGAAGAAAUAUA | |
| | AGACACCGGUCGCCACCAUGGAUAAG | |
| | AAAUACAGCAUUGGACUGGACAUUGG | |
| | GACAAACUCCGUGGGAUGGGCCGUGA | |
| | UUACAGACGAAUACAAAGUGCCUUCA | |
| | AAGAAGUUCAAGGUGCUGGGCAACAC | |
| | CGAUAGACACAGCAUCAAGAAAAAUC | |
| | UGAUUGGAGCCCUGCUGUUCGACUCC | |
| | GGCGAGACAGCuGAAGCAACUCGGCU | |
| | GAAAAGAACUGCUCGGAGAAGGUAUA | |
| | CCCGCCGAAAGAAUAGGAUCUGCUAC | |
| | CUGCAGGAGAUUUUCAGCAACGAAAU | |
| | GGCCAAGGUGGACGAUAGUUUCUUUC | |
| | ACCGCCUGGAGGAAUCAUUCCUGGUC | |
| | GAGGAAGAUAAGAAACACGAGCGGCA | |
| | UCCCAUCUUUGGCAACAUUGUGGACG | |
| | AGGUCGCUUAUCACGAAAAGUACCCU | |
| | ACCAUCUAUCAUCUGAGGAAGAAACU | |
| | GGUGGACUCCACAGAUAAAGCAGACC | |
| | UGCGCCUGAUCUAUCUGGCCCUGGCU | |
| | CACAUGAUUAAGUUCCGGGGCCAUUU | |
| | UCUGAUCGAGGGGGAUCUGAACCCAG | |
| | ACAAUUCUGAUGUGGACAAGCUGUUC | |
| | AUCCAGCUGGUCCAGACAUACAAUCA | |
| | GCUGUUUGAGGAAAACCCCAUUAAUG | |
| | CAUCUGGCGUGGACGCAAAAGCCAUC | |
| | CUGAGUGCCAGACUGUCUAAGAGUCG | |
| | GAGACUGGAGAACCUGAUCGCUCAGC | |
| | UGCCAGGGGAAAAGAAAAACGGCCUG | |
| | UUUGGGAAUCUGAUUGCACUGUCACU | |
| | GGGACUGACUCCCAACUUCAAGAGCA | |
| | AUUUUGAUCUGGCCGAGGACGCUAAA | |
| | CUGCAGCUGUCCAAGGACACCUAUGA | |
| | CGAUGACCUGGAUAACCUGCUGGCUC | |
| | AGAUCGGGGAUCAGUACGCAGACCUG | |
| | UUCCUGGCCGCUAAGAAUCUGUCUGA | |
| | CGCCAUCCUGCUGAGUGAUAUUCUGC | |
| | GCGUGAACACCGAGAUUACAAAAGCC | |
| | CCCCUGUCAGCUAGCAUGAUCAAGAG | |
| | AUAUGACGAGCACCAUCAGGAUCUGA | |
| | CCCUGCUGAAGGCUCUGGUGAGGCAG | |
| | CAGCUGCCUGAGAAGUACAAGGAAAU | |
| | CUUCUUUGAUCAGUCUAAGAACGGAU | |
| | ACGCCGGCUAUAUUGACGGCGGGGCU | |
| | AGUCAGGAGGAGUUCUACAAGUUUAU | |
| | CAAACCCAUUCUGGAGAAGAUGGAUG | |
| | GCACAGAGGAACUGCUGGUGAAACUG | |
| | AAUCGGGAAGACCUGCUGAGGAAGCA | |
| | GCGCACUUUUGAUAACGGAAGCAUCC | |
| | CUCACCAGAUUCAUCUGGGAGAGCUG | |
| | CACGCAAUCCUGAGGCGCCAGGAAGA | |
| | CUUCUACCCAUUUCUGAAGGAUAACA | |
| | GGGAGAAGAUCGAAAAAAUUCUGACA | |
| | UUCCGCAUCCCCUACUAUGUGGGCCC | |
| | UCUGGCAAGAGGCAACAGCCGGUUUG | |
| | CCUGGAUGACUCGCAAAUCUGAGGAA | |
| | ACAAUCACUCCCUGGAACUUCGAGGA | |
| | AGUGGUCGAUAAGGGCGCUUCCGCAC | |
| | AGUCUUUCAUUGAGCGGAUGACAAAC | |
| | UUCGACAAGAACCUGCCAAACGAAAA | |
| | AGUGCUGCCCAAGCACUCUCUGCUGU | |
| | ACGAGUAUUUCACAGUCUAUAACGAA | |
| | CUGACUAAGGUGAAAUACGUCACCGA | |
| | GGGGAUGAGAAAGCCUGCCUUCCUGA | |
| | GUGGAGAACAGAAGAAAGCUAUCGUG | |
| | GACCUGCUGUUUAAAACCAAUAGGAA | |
| | GGUGACAGUCAAGCAGCUGAAAGAGG | |
| | ACUAUUUCAAGAAAAUUGAAUGUUUC | |
| | GAUUCUGUGGAGAUCAGUGGCGUCGA | |
| | AGACAGGUUUAACGCCUCCCUGGGGA | |
| | CCUACCACGAUCUGCUGAAGAUCAUU | |
| | AAGGAUAAAGACUUCCUGGACAACGA | |
| | GGAAAAUGAGGAUAUCCUGGAAGACA | |
| | UUGUGCUGACCCUGACACUGUUUGAG | |
| | GAUAGGGAAAUGAUCGAGGAACGCCU | |
| | GAAGACCUAUGCCCAUCUGUUCGAUG | |
| | ACAAAGUGAUGAAACAGCUGAAGCGA | |
| | CGGAGAUACACAGGAUGGGGCCGACU | |
| | GUCUCGGAAGCUGAUCAAUGGGAUUC | |
| | GCGACAAACAGAGUGGAAAGACCAUC | |
| | CUGGACUUUCUGAAAUCAGAUGGCUU | |
| | CGCCAACCGGAACUUCAUGCAGCUGA | |
| | UUCACGAUGACAGCCUGACAUUCAAA | |
| | GAGGAUAUCCAGAAGGCACAGGUGUC | |
| | CGGGCAGGGAGACUCUCUGCACGAGC | |
| | AUAUCGCAAACCUGGCCGGCAGCCCU | |
| | GCCAUCAAGAAAGGGAUUCUGCAGAC | |
| | CGUGAAGGUGGUGGACGAGCUGGUGA | |
| | AAGUCAUGGGAAGACAUAAGCCAGAA | |
| | AACAUCGUGAUUGAGAUGGCCAGGGA | |
| | AAAUCAGACCACACAGAAAGGCCAGA | |
| | AGAACUCAAGGGAGCGCAUGAAAAGA | |
| | AUCGAGGAAGGAAUUAAGGAACUGGG | |
| | CAGCCAGAUCCUGAAAGAGCACCCCG | |
| | UGGAAAACACACAGCUGCAGAAUGAG | |
| | AAGCUGUAUCUGUACUAUCUGCAGAA | |
| | UGGACGCGAUAUGUACGUGGACCAGG | |
| | AGCUGGAUAUUAACCGACUGUCCGAU | |
| | UACGACGUGGAUCAUAUCGUCCCACA | |
| | GUCAUUCCUGAAAGAUGACAGCAUUG | |
| | ACAAUAAGGUGCUGACCCGCUCUGAC | |
| | AAAAACCGAGGCAAGAGUGAUAAUGU | |
| | CCCCUCAGAGGAAGUGGUCAAGAAAA | |
| | UGAAGAACUACUGGAGGCAGCUGCUG | |
| | AAUGCCAAACUGAUCACACAGCGAAA | |
| | GUUUGAUAACCUGACUAAAGCUGAGC | |
| | GGGGAGGCCUGAGUGAACUGGACAAA | |
| | GCAGGCUUCAUUAAGCGACAGCUGGU | |
| | GGAGACACGGCAGAUCACAAAGCACG | |

TABLE 12B
UCGCCCAGAUUCUGGAUUCAAGAAUGAACACUAAGUACGAUGAGAAUGA
CAAACUGAUCAGAGAAGUGAAGGUCAUUACCCUGAAGUCAAAACUGGUG
AGCGACUUUCGGAAAGAUUUCCAGUUUUAUAAGGUCAGAGAGAUCAACA
ACUACCACCAUGCUCAUGACGCAUACCUGAACGCAGUGGUCGGCACAGC
CCUGAUUAAGAAAUACCCUAAACUGGAGUCCGAGUUCGUGUACGGGGAC
UAUAAGGUGUACGAUGUCAGAAAAAUGAUCGCCAAGUCUGAGCAGGAAA
UUGGCAAAGCCACUGCUAAGUAUUUCUUUUACAGUAACAUCAUGAAUUU
CUUUAAGACUGAGAUCACCCUGGCAAAUGGGGAAAUCCGAAAGCGGCCA
CUGAUUGAGACUAACGGCGAGACAGGAGAAAUCGUGUGGGACAAAGGAA
GAGAUUUUCCUACCGUGAGGAAGGUCCUGAGCAUGCCCCAAGUGAAUAU
UGUCAAGAAAACAGAGGUGCAGACUGGGGGAUUCAGUAAGGAAUCAAUU
CUGCCUAAACGCAACUCCGAUAAGCUGAUCGCCCGAAAGAAAGACUGGG
ACCCCAAGAAGUAUGGCGGGUUCGACUCCCCAACUGUGGCUUACUCUGU
CCUGGUGGUCGCAAAGGUGGAGAAGGGAAAAAGCAAGAAACUGAAAUCC
GUCAAGGAACUGCUGGGCAUCACCAUUAUGGAGCGCAGCUCCUUCGAAA
AGAAUCCUAUCGAUUUUCUGGAGGCCAAAGGCUAUAAGGAAGUGAAGAA
AGACCUGAUCAUCAAGCUGCCAAAGUACUCACUGUUUGAGCUGGAAAAC
GGGAGAAAGAGGAUGCUGGCAAGCGCCGGGGAGCUGCAGAAAGGAAAUG
AACUGGCCCUGCCCUCCAAGUACGUGAACUUCCUGUAUCUGGCUAGCCA
CUACGAGAAGCUGAAAGGGUCCCCUGAGGAUAACGAACAGAAACAGCUG
UUUGUGGAGCAGCACAAGCAUUAUCUGGACGAGAUCAUUGAACAGAUUA
GCGAGUUCUCCAAAAGAGUGAUCCUGGCUGACGCAAAUCUGGAUAAGGU
CCUGAGCGCAUACAACAAACACCGGGAUAAGCCAAUCAGAGAGCAGGCC
GAAAAUAUCAUUCAUCUGUUCACUCUGACCAACCUGGGAGCCCCCGCAG
CCUUCAAGUAUUUUGACACUACCAUCGAUCGCAAACGAUACACAAGCAC
UAAGGAGGUGCUGGACGCUACCCUGAUUCAUCAGAGCAUUACUGGCCUG
UAUGAAACAAGGAUUGACCUGUCUCAGCUGGGCGGCGACUCCGGAGCUG
ACCCCAAGAAGAAGAGGAAGGUGUGAUAGUCUAGACCUUCUGCGGGGCU
UGCCUUCUGGCCAUGCCCUUCUUCUCUCCCUUGCACCUGUACCUCUUGG
UCUUUGAAUAAAGCCUGAGUAGGAAAAAAAAAAAAAAAAAAAAAAAAAA
AAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA

TABLE 13

| RNA | | Sequence |
|---|---|---|
| name | Sequence (5′ → 3′) | ID No. |
| L7Ae | GGGCGAAUUAAGAGAGAAAAGAAGAGUAAG | 37 |
| mRNA | AAGAAAUAUAAGACACCGGUCGCCACCAUG | |
| | UACGUGAGAUUUGAGGUUCCUGAGGACAUG | |
| | CAGAACGAAGCUCUGAGUCUGCUGGAGAAG | |
| | GUUAGGGAGAGCGGUAAGGUAAAGAAAGGU | |
| | ACCAACGAGACGACAAAGGCUGUGGAGAGG | |
| | GGACUGGCAAAGCUCGUUUACAUCGCAGAG | |
| | GAUGUUGACCCGCCUGAGAUCGUUGCUCAU | |
| | CUGCCCCUCCUCUGCGAGGAGAAGAAUGUG | |
| | CCGUACAUUUACGUUAAAAGCAAGAACGAC | |
| | CUUGGAAGGGCUGUGGGCAUUGAGGUGCCA | |
| | UGCGCUUCGGCAGCGAUAAUCAACGAGGGA | |
| | GAGCUGAGAAAGGAGCUUGGAAGCCUUGUG | |
| | GAGAAGAUUAAAGGCCUUCAGAAGAGAUCU | |
| | CAUAUGCAUCUCGAGUGAUAGUCUAGACCU | |
| | UCUGCGGGGCUUGCCUUCUGGCCAUGCCCU | |
| | UCUUCUCUCCCUUGCACCUGUACCUCUUGG | |
| | UCUUUGAAUAAAGCCUGAGUAGGAAAAAAA | |
| | AAAAAAAAAAAAAAAAAAAAAAAAAAAAAA | |
| | AAAAAAAAAAAAAAAAAAAAAAAAAAAAAA | |
| | AAAAAAAAAAAAAAAAAAAAAAAAAAAAAA | |
| | AAAAAAAAAAAAAAAAAAAAAAA | |
| miR-21- | GGUUCCUUAAUCGCGGAUCCUCAACAUCAG | 38 |
| responsive | UCUGAUAAGCUAAGAUCACACCGGUCGCCA | |
| L7Ae | CCAUGUACGUGAGAUUUGAGGUUCCUGAGG | |
| | ACAUGCAGAACGAAGCUCUGAGUCUGCUGG | |
| | AGAAGGUUAGGGAGAGCGGUAAGGUAAAGA | |
| | AAGGUACCAACGAGACGACAAAGGCUGUGG | |
| | AGAGGGGACUGGCAAAGCUCGUUUACAUCG | |
| | CAGAGGAUGUUGACCCGCCUGAGAUCGUUG | |
| | CUCAUCUGCCCCUCCUCUGCGAGGAGAAGA | |
| | AUGUGCCGUACAUUUACGUUAAAAGCAAGA | |
| | ACGACCUUGGAAGGGCUGUGGGCAUUGAGG | |
| | UGCCAUGCGCUUCGGCAGCGAUAAUCAACG | |
| | AGGGAGAGCUGAGAAAGGAGCUUGGAAGCC | |
| | UUGUGGAGAAGAUUAAAGGCCUUCAGAAGA | |
| | GAUCUCAUAUGCAUCUCGAGUGAUAGUCUA | |
| | GACCUUCUGCGGGGCUUGCCUUCUGGCCAU | |
| | GCCCUUCUUCUCUCCCUUGCACCUGUACCU | |
| | CUUGGUCUUUGAAUAAAGCCUGAGUAGGAA | |
| | AAAAAAAAAAAAAAAAAAAAAAAAAAAAAA | |
| | AAAAAAAAAAAAAAAAAAAAAAAAAAAAAA | |
| | AAAAAAAAAAAAAAAAAAAAAAAAAAAAAA | |
| | AAAAAAAAAAAAAAAAAAAAAAAAAAAA | |
| Tag BFP | GGGCGAAUUAAGAGAGAAAAGAAGAGUAAG | 39 |
| mRNA | AAGAAAUAUAAGACACCGGUCGCCACCAUG | |
| | GGAUCCAGCGAGCUGAUUAAGGAGAACAUG | |
| | CACAUGAAGCUGUACAUGGAGGGCACCGUG | |
| | GACAACCAUCACUUCAAGUGCACAUCCGAG | |
| | GGCGAAGGCAAGCCCUACGAGGGCACCCAG | |
| | ACCAUGAGAAUCAAGGUGGUCGAGGGCGGC | |
| | CCUCUCCCCUUCGCCUUCGACAUCCUGGCU | |
| | ACUAGCUUCCUCUACGGCAGCAAGACCUUC | |
| | AUCAACCACACCCAGGGCAUCCCCGACUUC | |
| | UUCAAGCAGUCCUUCCCUGAGGGCUUCACA | |
| | UGGGAGAGAGUCACCACAUACGAAGACGGG | |
| | GGCGUGCUGACCGCUACCCAGGACACCAGC | |
| | CUCCAGGACGGCUGCCUCAUCUACAACGUC | |
| | AAGAUCAGAGGGGUGAACUUCACAUCCAAC | |
| | GGCCCUGUGAUGCAGAAGAAAACACUCGGC | |
| | UGGGAGGCCUUCACCGAGACGCUGUACCCC | |
| | GCUGACGGCGGCCUGGAAGGCAGAAACGAC | |
| | AUGGCCCUGAAGCUCGUGGGCGGGAGCCAU | |
| | CUGAUCGCAAACAUCAAGACCACAUAUAGA | |
| | UCCAAGAAACCCGCUAAGAACCUCAAGAUG | |
| | CCUGGCGUCUACUAUGUGGACUACAGACUG | |
| | GAAAGAAUCAAGGAGGCCAACAACGAGACC | |
| | UACGUCGAGCAGCACGAGGUGGCAGUGGCC | |
| | AGAUACUGCGACCUCCCUAGCAAACUGGGG | |
| | CACAGAUCUCAUAUGCAUCUCGAGUGAUAG | |
| | UCUAGACCUUCUGCGGGGCUUGCCUUCUGG | |
| | CCAUGCCCUUCUUCUCUCCCUUGCACCUGU | |
| | ACCUCUUGGUCUUUGAAUAAAGCCUGAGUA | |
| | GGAAAAAAAAAAAAAAAAAAAAAAAAAAAA | |
| | AAAAAAAAAAAAAAAAAAAAAAAAAAAAAA | |
| | AAAAAAAAAAAAAAAAAAAAAAAAAAAAAA | |
| | AAAAAAAAAAAAAAAAAAAAAAAAAAAAAA | |
| | AA | |

HeLa_GFP cells are seeded in a 24-well plate at 5×10⁵ cells/well, 17 to 24 hours before transfection.

OFF conditions: control L7Ae mRNA 15 ng+kt-Cas9 mRNA (having a kt motif in the 5′ UTR) 5 ng+sgRNA 150 ng

ON conditions: miR-21-responsive L7Ae mRNA 15 ng+kt-Cas9 mRNA 5 ng+sgRNA 150 ng

Transfection was performed using a Stemfect transfection reagent according to the protocol. Three days after transfection, the measurement was performed with Accuri.

Evaluation of miRNA Expression Level

Three types of cells, HeLa-EGFP cells, iPS-EGFP cells, and mDA-EGFP cells were used. The measurement was performed using a TaqMan® MicroRNA Cells-to-C_{T}™ kit (Ambion, Inc.). Cell lysates were subjected to reverse transcription using has-miR-21-5p (Assay ID: 000397), 302a-5p (Assay ID: 002381) and RNU 6B (Assay ID: 001093) TaqMan probes (Applied Biosystems), respectively. qPCR was performed with StepOne Plus Real-Time PCR System (Applied Biosystems) by using a TaqMan probe. The target miRNA was normalized by RNU6B. In addition, mDA-EGFP cells were normalized to 1.

Evaluation of Cas9 Protein Expression Level

A Cas9 mRNA in the amount of 100 ng and an sgRNA in the amount of 300 ng were used. Twenty-four hours after transfection, PBS wash was performed, and the cells were lysed with 50 μL of M-PER cocktail (a mixture of M-PER Mammalian Protein Extraction Reagent [Thermo Fisher Scientific, Inc.], a protease inhibitor and PMSF) and collected. After shaking for 5 minutes, the solution was collected in a 1.5 mL tube. After centrifugation (12400 rpm, 4° C., 5 min), the supernatant was collected in a new 1.5 mL tube, and the protein concentration was measured by the BCA method. The protein solution was diluted to 0.5 mg/mL, and the protein was detected according to the protocol of Wes (ProteinSimple). GAPDH was used as a loading control. Primary antibody: a Cas9 antibody (50-fold diluted, Active Motif, Inc.), a GAPDH antibody (100-fold diluted, Santa Cruz Biotechnology, Inc.). Secondary antibody: an anti-mouse, anti-rabbit antibody (ProteinSimple).

Sequencing

The second PCR product (iPS-EGFP) obtained by the T7E1 assay was inserted into a pUC19 vector, and the sequence was then read by a sequencer. First, the primers used in the second PCR were phosphorylated with T4PNK, and the second PCR product was again subjected to PCR. Then, it was inserted into the pUC19 vector, and the sequence was determined using an M13 Fwd New primer, a T7E1 Fwd primer and a T7E1 Rev primer. Applies Biosystems 3500×L Genetic analyzer was used as a sequencer.

Evaluation of Gene Expression Level

HeLa-EGFP cells or iPS-EGFP cells were used. In addition, 100 ng of a Cas9 mRNA and 300 ng of an sgRNA were used. Total RNAs were extracted from each of the cells with Trizol (Thermo Fisher Scientific, Inc.) according to the protocol. Genomic DNAs were also removed by using a TURBO DNase inactivation kit (Ambion, Inc.). Samples (250 or 300 ng) subjected to the above-described treatment were reverse transcribed by using ReverTra Acr® qPCR RT Master Mix (Toyobo Co., Ltd.). qPCR was performed by using THUNDERBIRDR® SYBR® qPCR Mix (Toyobo Co., Ltd.). For the reaction, StepOne Plus Real-Time PCR System (Applied Biosystems) was used. The target mRNA was normalized with GAPDH. Further normalization was performed so that the gene expression in the control Cas9 mRNA was 1.

Evaluation of Transfection Efficiency

HeLa-EGFP cells and iPS-EGFP cells were used. A BFP mRNA in the amount of 100 ng and a Cy5-labeled sgRNA in the amount of 300 ng were used. Twenty-four hours after transfection, cells were washed three times with PBS. Thereafter, the cells were collected in a 1.5 mL tube (See the section of “Cas9 activity assay” for cell release) and measured by LSR.

Cas9 Activity Assay (sgRNA Modified with iPS-EGFP: 1, 2)

This was performed in the same way as in the section of “Cas9 activity assay” described above.

The results are shown in FIGS. 8 to 15. ON-system is a system in which the Cas9 activity is increased by an endogenous miRNA. FIG. 8 (a) is a conceptual illustration showing the outline of the ON-system. mRNA 1 (a sequence to which L7Ae, an RNA binding protein derived from an archaebacterium binds: encoding K-turn in the 5′ UTR) expressing a Cas9 is translationally regulated by mRNA 2 expressing L7Ae (the expression of which is regulated by an miRNA). FIG. 8(b) shows that the Cas9 activity was increased by an endogenous miR-21 of HeLa cells. That is, it indicates that the Cas9 activity was increased by the target miRNA (herein miR-21).

miRNA Expression Level

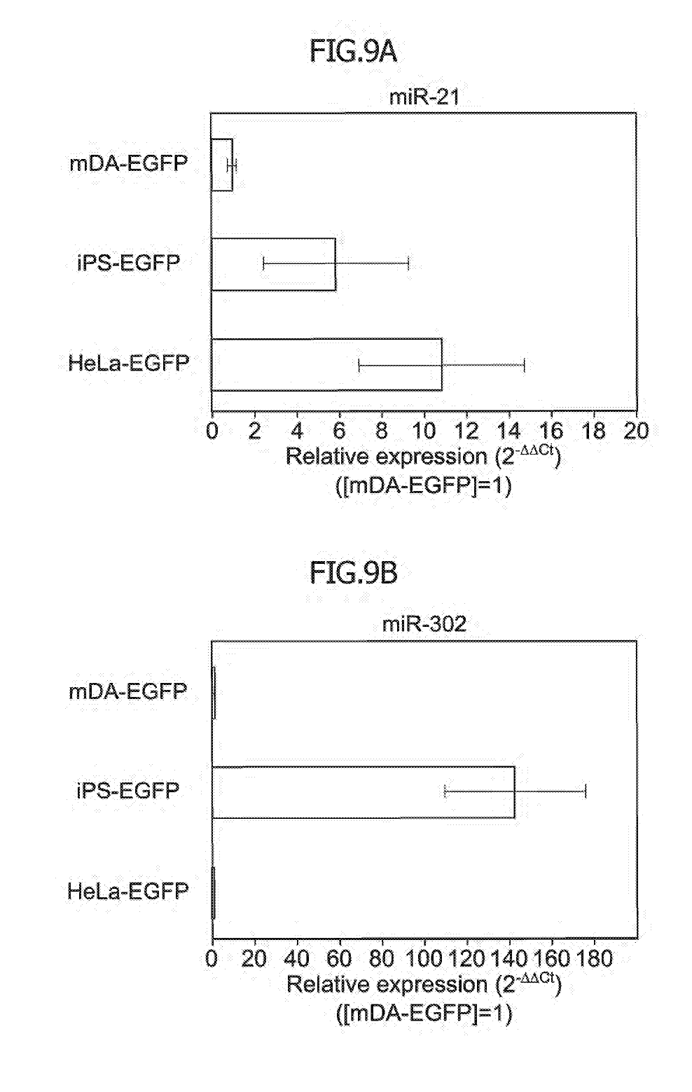

FIG. 9(a) shows the results of the expression levels of miR-21 and FIG. 9(b) shows the results of the expression levels of miR-302a, each of which levels was measured in HeLa-EGFP cells, iPS-EGFP cells and mDA-EGFP cells. The figures show that the expression of the miR-21 was high in the HeLa-EGFP cells and the expression of the miR-302a was high in the iPS-EGFP cells.

Evaluation of Cas9 Protein Expression Level

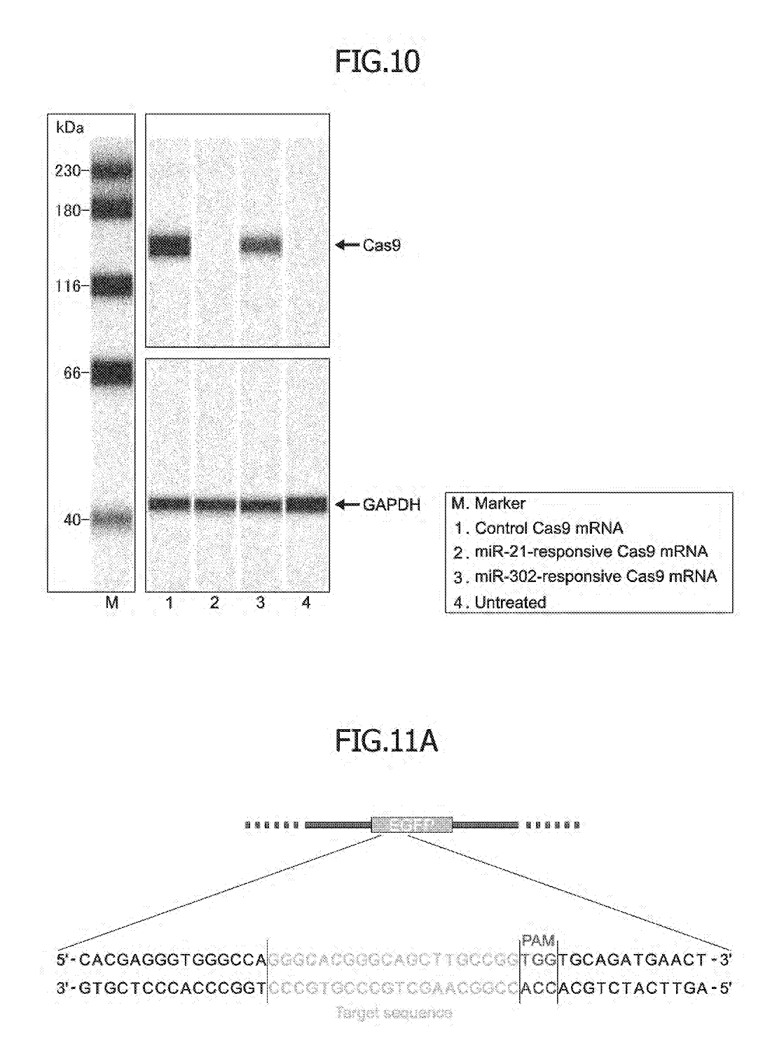

Each of a control Cas9 mRNA, miR-21-responsive Cas9 mRNA and miR-302a-responsive Cas9 mRNA was introduced into HeLa cells (also introducing an sgRNA together therewith), and it was examining by the Simple Western (Wes) assay whether each of them has an effect on the expression of a Cas9 protein. The results are shown in FIG. 10. The Cas9 protein was detected in the control and miR-302a-responsive Cas9 mRNA but not in the miR-21-responsive Cas9 mRNA. This indicates that little activity of the Cas9 protein was found in HeLa cells due to translational repression by miR-21 in the miRNA-21-responsive Cas9 mRNA. GAPDH was also used as a loading control.

Sequencing

The sequencing of the PCR products obtained by T7E1 shown in FIG. 4b) and FIG. 4c) were performed. FIG. 11(a) is a scheme showing a sequence around the target sequence. FIG. 11(b) shows that a mutation was introduced in the EGFP gene target region. That is, the decrease in the fluorescence intensity of EGFP indicates knockout of the EGFP gene by the CRISPR/Cas9 system.

In the figure, “Δ” represents the deletion of one or more bases and “+” indicates the insertion of one or more bases. Also, the number shown on the rightmost side represents the number of colonies of the obtained sequence/the total number of colonies.

Green letters represent a “target sequence”; blue letters (TGG and ACC in FIG. 11(a)) represent “PAM (protospacer adjuvant motif)”; purple letters (CCA in FIG. 11(b)” represent a “sequence complementary to PAM”; and red letters (for the control Cas9 mRNA, line 2: from the 5′ end the ninth and tenth bases and the twelfth to eighteenth bases (the latter expressed by “−”); line 3: from the 5′ end the nineteenth to the forty-first bases; and line 4: from the 5′ end the nineteenth to thirty-fifth bases; for the miR-302a-responsive Cas9 mRNA+miR-302 inhibitor, line 2: from the 5′ end the twentieth base; and line 3: from the 5′ end the seventeenth base (expressed by “−”)) represent a “mutation”, respectively. In the figure, the vertical lines between bases or between a base and “−” represent the boundary in which the letter color such as blue or red changes.

Evaluation of Gene Expression Level

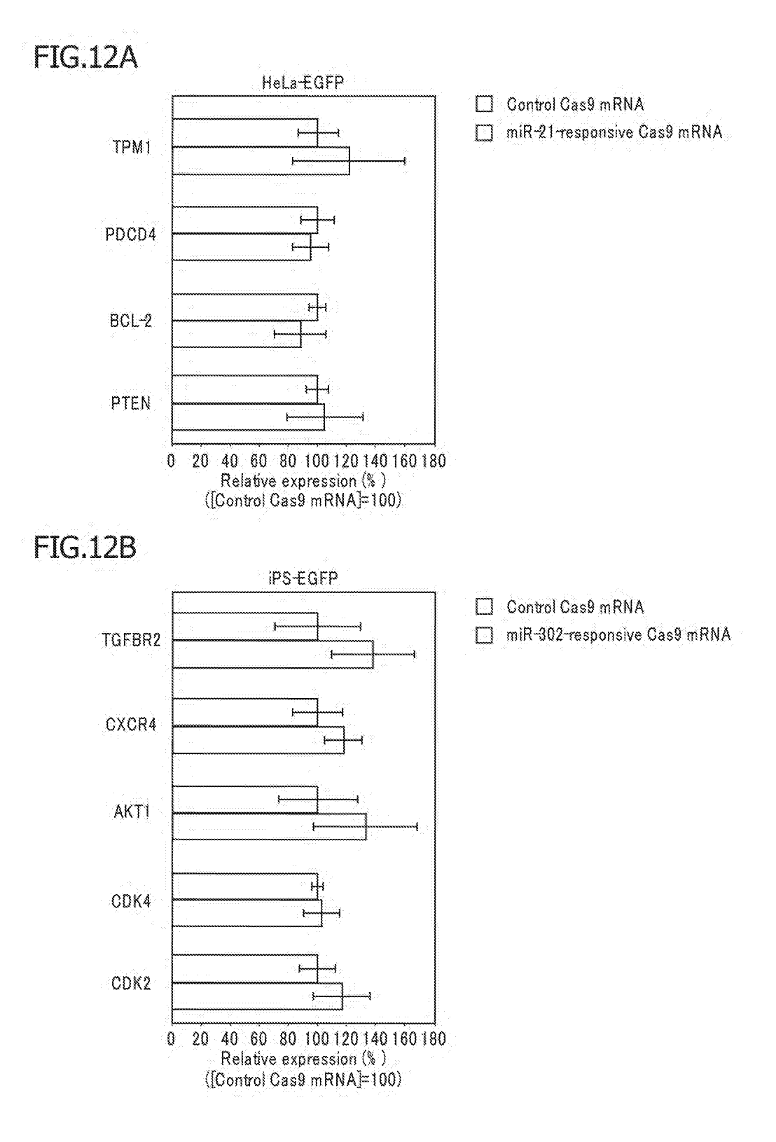

FIG. 12(a) and FIG. 12(b) show the results of the expression variation of genes the functions of which are known to be regulated by each of miR-21 and miR-302a using qPCR. Comparing the gene expression levels when a control Cas9 mRNA and an miRNA-responsive Cas9 mRNA were introduced into cells, there was little difference between the expression levels thereof. Thus, it indicates that the miRNA-responsive CRISPR/Cas9 system has little effect on the functions of the endogenous miR-21 and miR-302a.

Evaluation of Transfection Efficiency

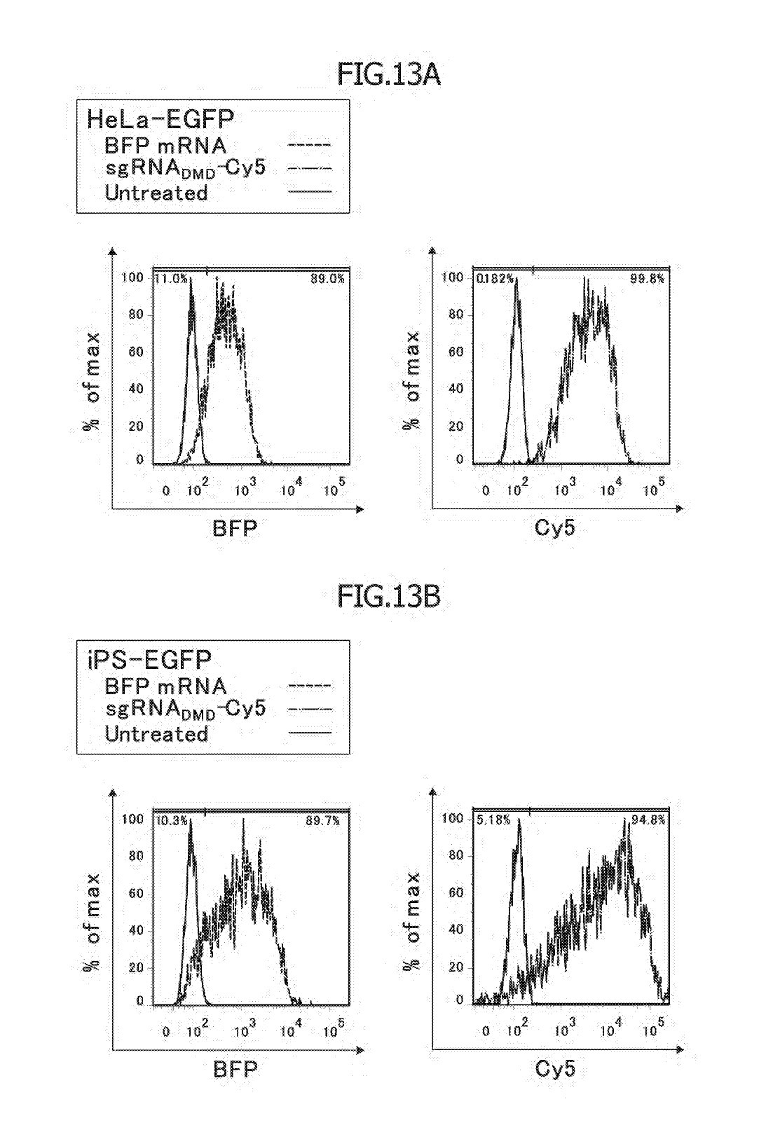

FIG. 13(a) shows the transfection efficiency of HeLa-EGFP cells, and FIG. 13(b) shows the transfection efficiency of iPS-EGFP cells. The transfection efficiency measured for each of a BFP mRNA and an sgRNA (labeled with Cy5) is shown. The transfection efficiency was approximately 90% or more.

EGFP Activity Assay (sgRNA Modified with iPS-EGFP: 1)

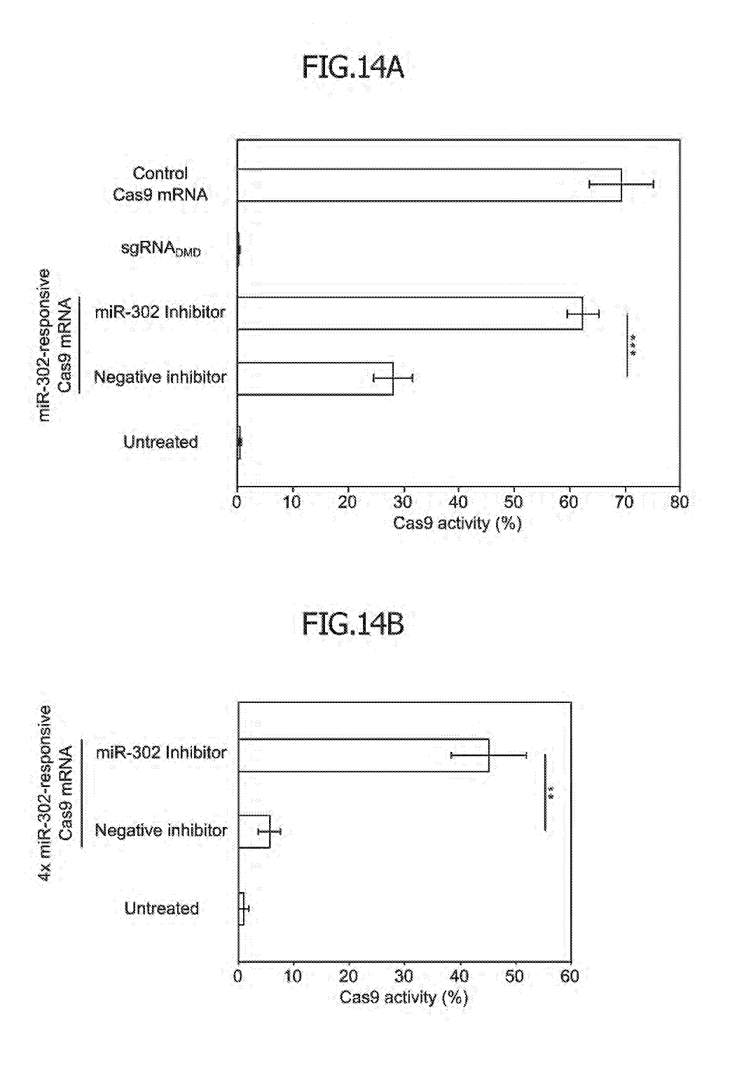

FIG. 14(a) shows the Cas9 activity when “GG” is deleted from the 5′ end of an EGFP-targeting sgRNA. The Cas9 activity of approximately 30% shown in FIG. 4 (a) increased to nearly 70%, indicating that the Cas9 activity was improved by modifying the guide strand. However, the Cas9 activity was near 30% even in the negative control for the inhibitor. In FIG. 14 (b), the miR-302-responsive Cas9 mRNA had been modified into a 4×miR-302-responsive Cas9 mRNA to reduce leakage of the Cas9 activity shown in FIG. 14 (a). It was found that using 50 ng of a Cas9 mRNA and 300 ng of an sgRNA maintained the high activity while further reducing the leakage.

Cas9 Activity Assay (sgRNA Modified with iPS-EGFP: 2)

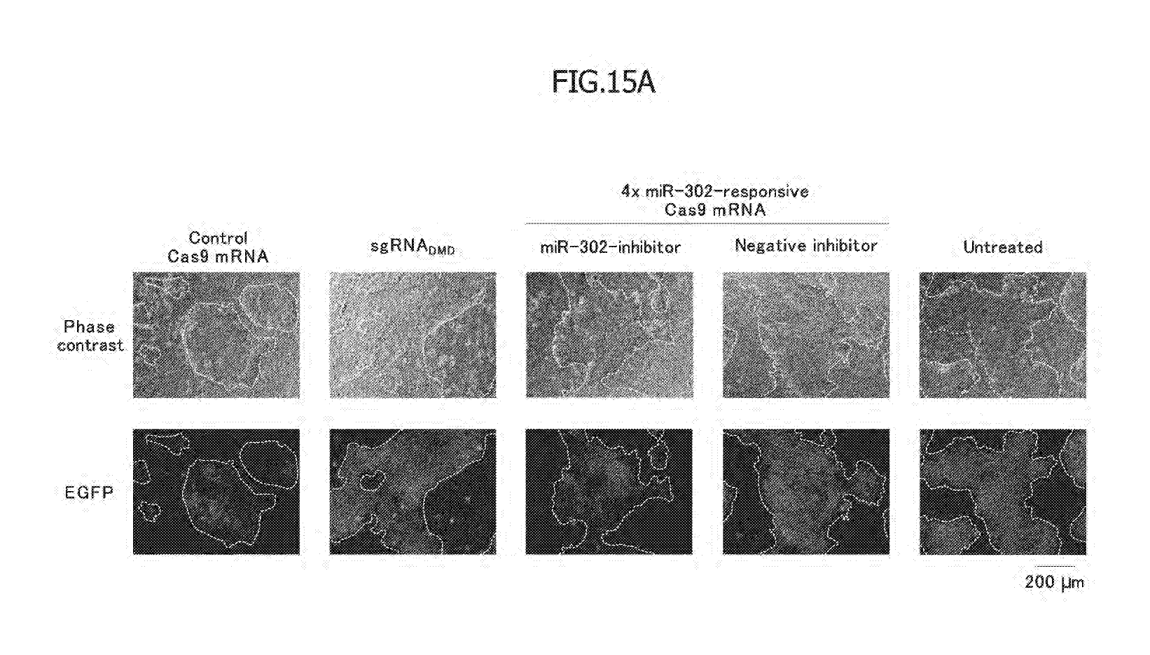

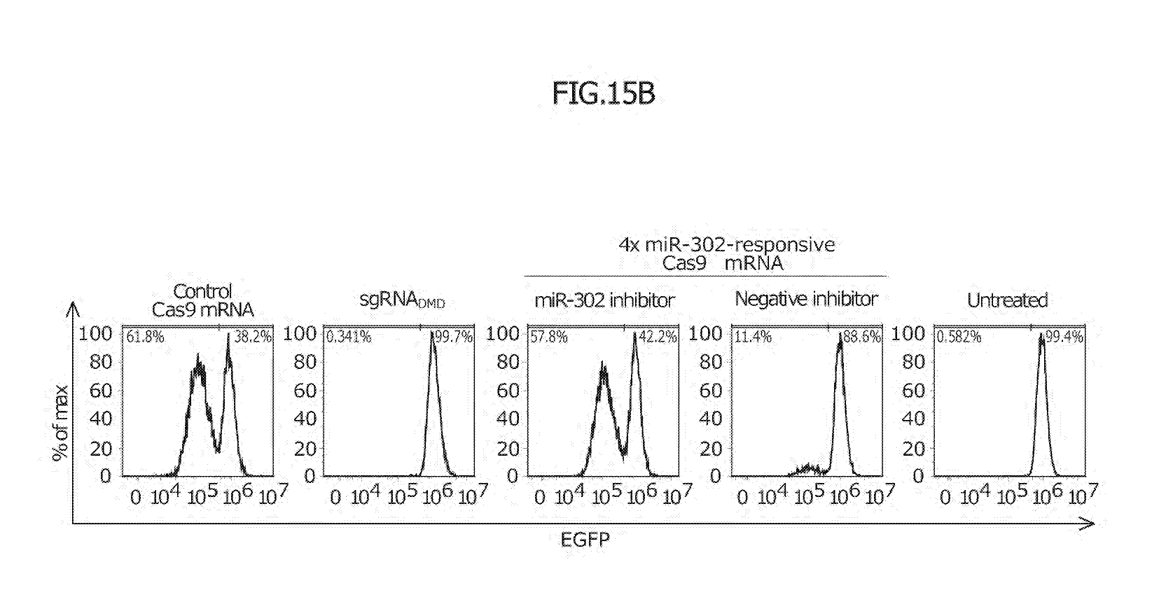

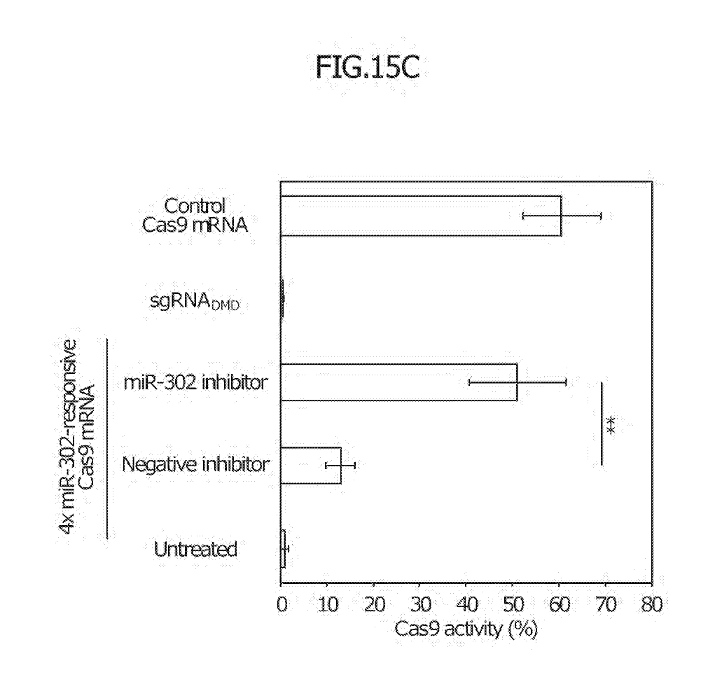

A Cas9 mRNA in the amount of 100 ng and an sgRNA in the amount of 300 ng were used. FIG. 15 shows the results obtained by modifying the miR-302-responsive Cas9 mRNA into a 4×miR-302-responsive Cas9 mRNA (having four sequences responding to miR-302 inserted in tandem in the 5′ UTR) to eliminate the leakage of the Cas9 activity seen in FIG. 14 (a) for the EGFP activity assay (sgRNA modified with iPS-EGFP: 1). FIG. 15(a) shows photographs of cells, FIG. 15(b) shows histograms for the photographs, and FIG. 15(c) shows the quantitative results. It was found that the leakage of the Cas9 activity was suppressed by increasing the number of sequences responding to miRNA.

1. A method for cell-specifically regulating a nuclease, comprising a step of introducing an miRNA-responsive mRNA encoding the nuclease into cells, wherein the miRNA-responsive mRNA comprises:
(i) a nucleic acid sequence specifically recognized by the miRNA; and
(ii) a nucleic acid sequence corresponding to a coding region for the nuclease.

2. The method according to claim 1, wherein the nucleic acid sequence specifically recognized by the miRNA is either an miR-302a-target sequence or an miR-21-target sequence.

3. The method according to claim 1, wherein the nuclease is a Cas9 protein or a variant thereof, and the method further comprises a step of introducing into the cells an sgRNA comprising a guide sequence specifically recognized by a target gene for the nuclease.

4. The method according to claim 3, for regulating the nuclease in a manner specific to undifferentiated cells.

5. The method according to claim 4, wherein the undifferentiated cells are cells expressing miR-302a.

6. The method according to claim 1, wherein the miRNA-responsive mRNA comprises a nucleic acid sequence having (i) and (ii) linked in a 5′ to 3′ direction.

7. An miRNA-responsive mRNA, comprising:
(i) a nucleic acid sequence specifically recognized by the miRNA; and
(ii) a nucleic acid sequence corresponding to a coding region for a nuclease.

8. The miRNA-responsive mRNA according to claim 7, wherein the nucleic acid sequence specifically recognized by the miRNA is either an miR-302a-target sequence or an miR-21-target sequence.

9. The miRNA-responsive mRNA according to claim 7, wherein the nuclease is a Cas9 protein or a variant thereof.

10. A kit for cell-specifically regulating a nuclease, comprising:
the miRNA-responsive mRNA according to claim 9; and
an sgRNA comprising a guide sequence specifically recognized by a target gene for the nuclease.

11. A method for cell-specifically regulating a nuclease, comprising a step of introducing into cells
a) a trigger protein-responsive mRNA encoding the nuclease; and
b) an miRNA-responsive mRNA encoding the trigger protein,
wherein the protein-responsive mRNA encoding the nuclease a) comprises:
(ia) a nucleic acid sequence that specifically hinds to the protein; and
(iia) a nucleic acid sequence corresponding to a coding region for the nuclease; and
wherein the miRNA-responsive mRNA encoding the trigger protein b) comprises:
(ib) a nucleic acid sequence specifically recognized by the miRNA; and
(iib) a nucleic acid sequence corresponding to a coding region for the trigger protein.

12. The method according to claim 11, wherein the trigger protein comprises an L7Ae protein or a derivative thereof, and the nucleic acid sequence that specifically hinds to the protein comprises a K-turn sequence or a derivative thereof.

13. The method according to claim 11, wherein the nucleic acid sequence specifically recognized by the miRNA is either an miR-302a-target sequence or an miR-21-target sequence.

14. The method according to claim 11, wherein the nuclease is a Cas9 protein or a variant thereof, and the method further comprises a step of introducing into the cell an sgRNA comprising a guide sequence specifically recognized by a target gene for the nuclease.

15. The method according to claim 14, for regulating the nuclease in a manner specific to undifferentiated cells.

16. The method according to claim 15, wherein the undifferentiated cells are cells expressing miR-302a.

17. The method according to claim 11, wherein:
the a) protein-responsive mRNA encoding the trigger protein comprises a nucleic acid sequence having (ia) and (iia) linked in a 5′ to 3′ direction; and
the h) miRNA-responsive mRNA encoding the nuclease comprises a nucleic acid sequence having (ib) and (iib) linked in a 5′ to 3′ direction.

18. A nuclease regulator comprising:
a) a trigger protein-responsive mRNA encoding a nuclease, comprising:
(ia) a nucleic acid sequence that specifically binds to the protein; and
(iia) a nucleic acid sequence corresponding to a coding region for the nuclease; and
b) an miRNA-responsive mRNA encoding the trigger protein comprising:
(ib) a nucleic acid sequence specifically recognized by the miRNA; and
(iib) a nucleic acid sequence corresponding to, a coding region for the trigger protein.

19. The nuclease regulator according to claim 18, wherein the trigger protein comprises an L7Ae protein or a derivative thereof and the nucleic acid sequence that specifically binds to the protein comprises a K-turn sequence or a derivative thereof.

20. The nuclease regulator according, to claim 18, wherein in the miRNA-responsive mRNA encoding the trigger protein, the nucleic acid sequence specifically recognized by the miRNA is either an miR-302a-target sequence or an miR-21-target sequence.

21. The nuclease regulator according to claim 18, wherein in the trigger protein-responsive mRNA, the nuclease is a Cas9 protein or a variant thereof.

22. A kit for cell-specifically regulating a nuclease, comprising:
the nuclease regulator according to claim 18; and
an sgRNA comprising a guide sequence specifically recognized by a target gene for the nuclease.


SEQUENCE LISTING (44 sequences)

SEQ ID NO: 1 - RNA, 23 nt, Artificial Sequence
  miR-302a
acuuaaacguggauguacuugcu

SEQ ID NO: 2 - RNA, 22 nt, Artificial Sequence
  miR-21
uagcuuaucagacugauguuga

SEQ ID NO: 3 - RNA, 23 nt, Artificial Sequence
  Tg miR-302a
agcaaguacauccacguuuaagu

SEQ ID NO: 4 - RNA, 22 nt, Artificial Sequence
  Tg miR-21
ucaacaucagucugauaagcua

SEQ ID NO: 5 - RNA, 4417 nt, Artificial Sequence
  miR-302a responsive Cas 9 mRNA
gguuccuuaaucgcggauccagcaaguacauccacguuuaaguagauccaccggucgccaccauggauaagaaauacagcauuggacuggacauugggacaaacuccgugggaugggccgugauuacagacgaauacaaagugccuucaaagaaguucaaggugcugggcaacaccgauagacacagcaucaagaaaaaucugauuggagcccugcuguucgacuccggcgagacagcugaagcaacucggcugaaaagaacugcucggagaagguauacccgccgaaagaauaggaucugcuaccugcaggagauuuucagcaacgaaauggccaagguggacgauaguuucuuucaccgccuggaggaaucauuccuggucgaggaagauaagaaacacgagcggcaucccaucuuuggcaacauuguggacgaggucgcuuaucacgaaaaguacccuaccaucuaucaucugaggaagaaacugguggacuccacagauaaagcagaccugcgccugaucuaucuggcccuggcucacaugauuaaguuccggggccauuuucugaucgagggggaucugaacccagacaauucugauguggacaagcuguucauccagcugguccagacauacaaucagcuguuugaggaaaaccccauuaaugcaucuggcguggacgcaaaagccauccugagugccagacugucuaagagucggagacuggagaaccugaucgcucagcugccaggggaaaagaaaaacggccuguuugggaaucugauugcacugucacugggacugacucccaacuucaagagcaauuuugaucuggccgaggacgcuaaacugcagcuguccaaggacaccuaugacgaugaccuggauaaccugcuggcucagaucggggaucaguacgcagaccuguuccuggccgcuaagaaucugucugacgccauccugcugagugauauucugcgcgugaacaccgagauuacaaaagccccccugucagcuagcaugaucaagagauaugacgagcaccaucaggaucugacccugcugaaggcucuggugaggcagcagcugccugagaaguacaaggaaaucuucuuugaucagucuaagaacggauacgccggcuauauugacggcggggcuagucaggaggaguucuacaaguuuaucaaacccauucuggagaagauggauggcacagaggaacugcuggugaaacugaaucgggaagaccugcugaggaagcagcgcacuuuugauaacggaagcaucccucaccagauucaucugggagagcugcacgcaauccugaggcgccaggaagacuucuacccauuucugaaggauaacagggagaagaucgaaaaaauucugacauuccgcauccccuacuaugugggcccucuggcaagaggcaacagccgguuugccuggaugacucgcaaaucugaggaaacaaucacucccuggaacuucgaggaaguggucgauaagggcgcuuccgcacagucuuucauugagcggaugacaaacuucgacaagaaccugccaaacgaaaaagugcugcccaagcacucucugcuguacgaguauuucacagucuauaacgaacugacuaaggugaaauacgucaccgaggggaugagaaagccugccuuccugaguggagaacagaagaaagcuaucguggaccugcuguuuaaaaccaauaggaaggugacagucaagcagcugaaagaggacuauuucaagaaaauugaauguuucgauucuguggagaucaguggcgucgaagacagguuuaacgccucccuggggaccuaccacgaucugcugaagaucauuaaggauaaagacuuccuggacaacgaggaaaaugaggauauccuggaagacauugugcugacccugacacuguuugaggauagggaaaugaucgaggaacgccugaagaccuaugcccaucuguucgaugacaaagugaugaaacagcugaagcgacggagauacacaggauggggccgacugucucggaagcugaucaaugggauucgcgacaaacagaguggaaagaccauccuggacuuucugaaaucagauggcuucgccaaccggaacuucaugcagcugauucacgaugacagccugacauucaaagaggauauccagaaggcacagguguccgggcagggagacucucugcacgagcauaucgcaaaccuggccggcagcccugccaucaagaaagggauucugcagaccgugaaggugguggacgagcuggugaaagucaugggaagacauaagccagaaaacaucgugauugagauggccagggaaaaucagaccacacagaaaggccagaagaacucaagggagcgcaugaaaagaaucgaggaaggaauuaaggaacugggcagccagauccugaaagagcaccccguggaaaacacacagcugcagaaugagaagcuguaucuguacuaucugcagaauggacgcgauauguacguggaccaggagcuggauauuaaccgacuguccgauuacgacguggaucauaucgucccacagucauuccugaaagaugacagcauugacaauaaggugcugacccgcucugacaaaaaccgaggcaagagugauaauguccccucagaggaaguggucaagaaaaugaagaacuacuggaggcagcugcugaaugccaaacugaucacacagcgaaaguuugauaaccugacuaaagcugagcggggaggccugagugaacuggacaaagcaggcuucauuaagcgacagcugguggagacacggcagaucacaaagcacgucgcccagauucuggauucaagaaugaacacuaaguacgaugagaaugacaaacugaucagagaagugaaggucauuacccugaagucaaaacuggugagcgacuuucggaaagauuuccaguuuuauaaggucagagagaucaacaacuaccaccaugcucaugacgcauaccugaacgcaguggucggcacagcccugauuaagaaauacccuaaacuggaguccgaguucguguacggggacuauaagguguacgaugucagaaaaaugaucgccaagucugagcaggaaauuggcaaagccacugcuaaguauuucuuuuacaguaacaucaugaauuucuuuaagacugagaucacccuggcaaauggggaaauccgaaagcggccacugauugagacuaacggcgagacaggagaaaucgugugggacaaaggaagagauuuugcuaccgugaggaagguccugagcaugccccaagugaauauugucaagaaaacagaggugcagacugggggauucaguaaggaaucaauucugccuaaacgcaacuccgauaagcugaucgcccgaaagaaagacugggaccccaagaaguauggcggguucgacuccccaacuguggcuuacucuguccugguggucgcaaagguggagaagggaaaaagcaagaaacugaaauccgucaaggaacugcugggcaucaccauuauggagcgcagcuccuucgaaaagaauccuaucgauuuucuggaggccaaaggcuauaaggaagugaagaaagaccugaucaucaagcugccaaaguacucacuguuugagcuggaaaacgggagaaagaggaugcuggcaagcgccggggagcugcagaaaggaaaugaacuggcccugcccuccaaguacgugaacuuccuguaucuggcuagccacuacgagaagcugaaaggguccccugaggauaacgaacagaaacagcuguuuguggagcagcacaagcauuaucuggacgagaucauugaacagauuagcgaguucuccaaaagagugauccuggcugacgcaaaucuggauaagguccugagcgcauacaacaaacaccgggauaagccaaucagagagcaggccgaaaauaucauucaucuguucacucugaccaaccugggagcccccgcagccuucaaguauuuugacacuaccaucgaucgcaaacgauacacaagcacuaaggaggugcuggacgcuacccugauucaucagagcauuacuggccuguaugaaacaaggauugaccugucucagcugggcggcgacuccggagcugaccccaagaagaagaggaaggugugauagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 4417 nt, Artificial Sequence
  miR-21 responsive Cas 9 mRNA
gguuccuuaaucgcggauccucaacaucagucugauaagcuaagaucacaccggucgccaccauggauaagaaauacagcauuggacuggacauugggacaaacuccgugggaugggccgugauuacagacgaauacaaagugccuucaaagaaguucaaggugcugggcaacaccgauagacacagcaucaagaaaaaucugauuggagcccugcuguucgacuccggcgagacagcugaagcaacucggcugaaaagaacugcucggagaagguauacccgccgaaagaauaggaucugcuaccugcaggagauuuucagcaacgaaauggccaagguggacgauaguuucuuucaccgccuggaggaaucauuccuggucgaggaagauaagaaacacgagcggcaucccaucuuuggcaacauuguggacgaggucgcuuaucacgaaaaguacccuaccaucuaucaucugaggaagaaacugguggacuccacagauaaagcagaccugcgccugaucuaucuggcccuggcucacaugauuaaguuccggggccauuuucugaucgagggggaucugaacccagacaauucugauguggacaagcuguucauccagcugguccagacauacaaucagcuguuugaggaaaaccccauuaaugcaucuggcguggacgcaaaagccauccugagugccagacugucuaagagucggagacuggagaaccugaucgcucagcugccaggggaaaagaaaaacggccuguuugggaaucugauugcacugucacugggacugacucccaacuucaagagcaauuuugaucuggccgaggacgcuaaacugcagcuguccaaggacaccuaugacgaugaccuggauaaccugcuggcucagaucggggaucaguacgcagaccuguuccuggccgcuaagaaucugucugacgccauccugcugagugauauucugcgcgugaacaccgagauuacaaaagccccccugucagcuagcaugaucaagagauaugacgagcaccaucaggaucugacccugcugaaggcucuggugaggcagcagcugccugagaaguacaaggaaaucuucuuugaucagucuaagaacggauacgccggcuauauugacggcggggcuagucaggaggaguucuacaaguuuaucaaacccauucuggagaagauggauggcacagaggaacugcuggugaaacugaaucgggaagaccugcugaggaagcagcgcacuuuugauaacggaagcaucccucaccagauucaucugggagagcugcacgcaauccugaggcgccaggaagacuucuacccauuucugaaggauaacagggagaagaucgaaaaaauucugacauuccgcauccccuacuaugugggcccucuggcaagaggcaacagccgguuugccuggaugacucgcaaaucugaggaaacaaucacucccuggaacuucgaggaaguggucgauaagggcgcuuccgcacagucuuucauugagcggaugacaaacuucgacaagaaccugccaaacgaaaaagugcugcccaagcacucucugcuguacgaguauuucacagucuauaacgaacugacuaaggugaaauacgucaccgaggggaugagaaagccugccuuccugaguggagaacagaagaaagcuaucguggaccugcuguuuaaaaccaauaggaaggugacagucaagcagcugaaagaggacuauuucaagaaaauugaauguuucgauucuguggagaucaguggcgucgaagacagguuuaacgccucccuggggaccuaccacgaucugcugaagaucauuaaggauaaagacuuccuggacaacgaggaaaaugaggauauccuggaagacauugugcugacccugacacuguuugaggauagggaaaugaucgaggaacgccugaagaccuaugcccaucuguucgaugacaaagugaugaaacagcugaagcgacggagauacacaggauggggccgacugucucggaagcugaucaaugggauucgcgacaaacagaguggaaagaccauccuggacuuucugaaaucagauggcuucgccaaccggaacuucaugcagcugauucacgaugacagccugacauucaaagaggauauccagaaggcacagguguccgggcagggagacucucugcacgagcauaucgcaaaccuggccggcagcccugccaucaagaaagggauucugcagaccgugaaggugguggacgagcuggugaaagucaugggaagacauaagccagaaaacaucgugauugagauggccagggaaaaucagaccacacagaaaggccagaagaacucaagggagcgcaugaaaagaaucgaggaaggaauuaaggaacugggcagccagauccugaaagagcaccccguggaaaacacacagcugcagaaugagaagcuguaucuguacuaucugcagaauggacgcgauauguacguggaccaggagcuggauauuaaccgacuguccgauuacgacguggaucauaucgucccacagucauuccugaaagaugacagcauugacaauaaggugcugacccgcucugacaaaaaccgaggcaagagugauaauguccccucagaggaaguggucaagaaaaugaagaacuacuggaggcagcugcugaaugccaaacugaucacacagcgaaaguuugauaaccugacuaaagcugagcggggaggccugagugaacuggacaaagcaggcuucauuaagcgacagcugguggagacacggcagaucacaaagcacgucgcccagauucuggauucaagaaugaacacuaaguacgaugagaaugacaaacugaucagagaagugaaggucauuacccugaagucaaaacuggugagcgacuuucggaaagauuuccaguuuuauaaggucagagagaucaacaacuaccaccaugcucaugacgcauaccugaacgcaguggucggcacagcccugauuaagaaauacccuaaacuggaguccgaguucguguacggggacuauaagguguacgaugucagaaaaaugaucgccaagucugagcaggaaauuggcaaagccacugcuaaguauuucuuuuacaguaacaucaugaauuucuuuaagacugagaucacccuggcaaauggggaaauccgaaagcggccacugauugagacuaacggcgagacaggagaaaucgugugggacaaaggaagagauuuugcuaccgugaggaagguccugagcaugccccaagugaauauugucaagaaaacagaggugcagacugggggauucaguaaggaaucaauucugccuaaacgcaacuccgauaagcugaucgcccgaaagaaagacugggaccccaagaaguauggcggguucgacuccccaacuguggcuuacucuguccugguggucgcaaagguggagaagggaaaaagcaagaaacugaaauccgucaaggaacugcugggcaucaccauuauggagcgcagcuccuucgaaaagaauccuaucgauuuucuggaggccaaaggcuauaaggaagugaagaaagaccugaucaucaagcugccaaaguacucacuguuugagcuggaaaacgggagaaagaggaugcuggcaagcgccggggagcugcagaaaggaaaugaacuggcccugcccuccaaguacgugaacuuccuguaucuggcuagccacuacgagaagcugaaaggguccccugaggauaacgaacagaaacagcuguuuguggagcagcacaagcauuaucuggacgagaucauugaacagauuagcgaguucuccaaaagagugauccuggcugacgcaaaucuggauaagguccugagcgcauacaacaaacaccgggauaagccaaucagagagcaggccgaaaauaucauucaucuguucacucugaccaaccugggagcccccgcagccuucaaguauuuugacacuaccaucgaucgcaaacgauacacaagcacuaaggaggugcuggacgcuacccugauucaucagagcauuacuggccuguaugaaacaaggauugaccugucucagcugggcggcgacuccggagcugaccccaagaagaagaggaaggugugauagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - RNA, 4487 nt, Artificial Sequence
  4 x miR-302a responsive Cas 9 mRNA
gguuccuuaaucgcggauccagcaaguacauccacguuuaaguagcaaguacauccacguuuaaguagcaaguacauccacguuuaaguagcaaguacauccacguuuaaguagaucacaccggucgccaccauggauaagaaauacagcauuggacuggacauugggacaaacuccgugggaugggccgugauuacagacgaauacaaagugccuucaaagaaguucaaggugcugggcaacaccgauagacacagcaucaagaaaaaucugauuggagcccugcuguucgacuccggcgagacagcugaagcaacucggcugaaaagaacugcucggagaagguauacccgccgaaagaauaggaucugcuaccugcaggagauuuucagcaacgaaauggccaagguggacgauaguuucuuucaccgccuggaggaaucauuccuggucgaggaagauaagaaacacgagcggcaucccaucuuuggcaacauuguggacgaggucgcuuaucacgaaaaguacccuaccaucuaucaucugaggaagaaacugguggacuccacagauaaagcagaccugcgccugaucuaucuggcccuggcucacaugauuaaguuccggggccauuuucugaucgagggggaucugaacccagacaauucugauguggacaagcuguucauccagcugguccagacauacaaucagcuguuugaggaaaaccccauuaaugcaucuggcguggacgcaaaagccauccugagugccagacugucuaagagucggagacuggagaaccugaucgcucagcugccaggggaaaagaaaaacggccuguuugggaaucugauugcacugucacugggacugacucccaacuucaagagcaauuuugaucuggccgaggacgcuaaacugcagcuguccaaggacaccuaugacgaugaccuggauaaccugcuggcucagaucggggaucaguacgcagaccuguuccuggccgcuaagaaucugucugacgccauccugcugagugauauucugcgcgugaacaccgagauuacaaaagccccccugucagcuagcaugaucaagagauaugacgagcaccaucaggaucugacccugcugaaggcucuggugaggcagcagcugccugagaaguacaaggaaaucuucuuugaucagucuaagaacggauacgccggcuauauugacggcggggcuagucaggaggaguucuacaaguuuaucaaacccauucuggagaagauggauggcacagaggaacugcuggugaaacugaaucgggaagaccugcugaggaagcagcgcacuuuugauaacggaagcaucccucaccagauucaucugggagagcugcacgcaauccugaggcgccaggaagacuucuacccauuucugaaggauaacagggagaagaucgaaaaaauucugacauuccgcauccccuacuaugugggcccucuggcaagaggcaacagccgguuugccuggaugacucgcaaaucugaggaaacaaucacucccuggaacuucgaggaaguggucgauaagggcgcuuccgcacagucuuucauugagcggaugacaaacuucgacaagaaccugccaaacgaaaaagugcugcccaagcacucucugcuguacgaguauuucacagucuauaacgaacugacuaaggugaaauacgucaccgaggggaugagaaagccugccuuccugaguggagaacagaagaaagcuaucguggaccugcuguuuaaaaccaauaggaaggugacagucaagcagcugaaagaggacuauuucaagaaaauugaauguuucgauucuguggagaucaguggcgucgaagacagguuuaacgccucccuggggaccuaccacgaucugcugaagaucauuaaggauaaagacuuccuggacaacgaggaaaaugaggauauccuggaagacauugugcugacccugacacuguuugaggauagggaaaugaucgaggaacgccugaagaccuaugcccaucuguucgaugacaaagugaugaaacagcugaagcgacggagauacacaggauggggccgacugucucggaagcugaucaaugggauucgcgacaaacagaguggaaagaccauccuggacuuucugaaaucagauggcuucgccaaccggaacuucaugcagcugauucacgaugacagccugacauucaaagaggauauccagaaggcacagguguccgggcagggagacucucugcacgagcauaucgcaaaccuggccggcagcccugccaucaagaaagggauucugcagaccgugaaggugguggacgagcuggugaaagucaugggaagacauaagccagaaaacaucgugauugagauggccagggaaaaucagaccacacagaaaggccagaagaacucaagggagcgcaugaaaagaaucgaggaaggaauuaaggaacugggcagccagauccugaaagagcaccccguggaaaacacacagcugcagaaugagaagcuguaucuguacuaucugcagaauggacgcgauauguacguggaccaggagcuggauauuaaccgacuguccgauuacgacguggaucauaucgucccacagucauuccugaaagaugacagcauugacaauaaggugcugacccgcucugacaaaaaccgaggcaagagugauaauguccccucagaggaaguggucaagaaaaugaagaacuacuggaggcagcugcugaaugccaaacugaucacacagcgaaaguuugauaaccugacuaaagcugagcggggaggccugagugaacuggacaaagcaggcuucauuaagcgacagcugguggagacacggcagaucacaaagcacgucgcccagauucuggauucaagaaugaacacuaaguacgaugagaaugacaaacugaucagagaagugaaggucauuacccugaagucaaaacuggugagcgacuuucggaaagauuuccaguuuuauaaggucagagagaucaacaacuaccaccaugcucaugacgcauaccugaacgcaguggucggcacagcccugauuaagaaauacccuaaacuggaguccgaguucguguacggggacuauaagguguacgaugucagaaaaaugaucgccaagucugagcaggaaauuggcaaagccacugcuaaguauuucuuuuacaguaacaucaugaauuucuuuaagacugagaucacccuggcaaauggggaaauccgaaagcggccacugauugagacuaacggcgagacaggagaaaucgugugggacaaaggaagagauuuugcuaccgugaggaagguccugagcaugccccaagugaauauugucaagaaaacagaggugcagacugggggauucaguaaggaaucaauucugccuaaacgcaacuccgauaagcugaucgcccgaaagaaagacugggaccccaagaaguauggcggguucgacuccccaacuguggcuuacucuguccugguggucgcaaagguggagaagggaaaaagcaagaaacugaaauccgucaaggaacugcugggcaucaccauuauggagcgcagcuccuucgaaaagaauccuaucgauuuucuggaggccaaaggcuauaaggaagugaagaaagaccugaucaucaagcugccaaaguacucacuguuugagcuggaaaacgggagaaagaggaugcuggcaagcgccggggagcugcagaaaggaaaugaacuggcccugcccuccaaguacgugaacuuccuguaucuggcuagccacuacgagaagcugaaaggguccccugaggauaacgaacagaaacagcuguuuguggagcagcacaagcauuaucuggacgagaucauugaacagauuagcgaguucuccaaaagagugauccuggcugacgcaaaucuggauaagguccugagcgcauacaacaaacaccgggauaagccaaucagagagcaggccgaaaauaucauucaucuguucacucugaccaaccugggagcccccgcagccuucaaguauuuugacacuaccaucgaucgcaaacgauacacaagcacuaaggaggugcuggacgcuacccugauucaucagagcauuacuggccuguaugaaacaaggauugaccugucucagcugggcggcgacuccggagcugaccccaagaagaagaggaaggugugauagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - RNA, 98 nt, Artificial Sequence
  sgRNA_DMD
gguaucuuacaggaacuccguuuuagagcuagaaauagcaaguuaaaauaaggcuaguccguuaucaacuugaaaaaguggcaccgagucggugcuuu

SEQ ID NO: 9 - RNA, 101 nt, Artificial Sequence
  sgRNA_ Alu1
gggccuguaaucccagcacuuuguuuuagagcuagaaauagcaaguuaaaauaaggcuaguccguuaucaacuugaaaaaguggcaccgagucggugcuuu

SEQ ID NO: 10 - RNA, 101 nt, Artificial Sequence
  sgRNA_ EGFP
gggggcacgggcagcuugccggguuuuagagcuagaaauagcaaguuaaaauaaggcuaguccguuaucaacuugaaaaaguggcaccgagucggugcuuu

SEQ ID NO: 11 - RNA, 99 nt, Artificial Sequence
  sgRNA_ EGFP(2)
gggcacgggcagcuugccggguuuuagagcuagaaauagcaaguuaaaauaaggcuaguccguuaucaacuugaaaaaguggcaccgagucggugcuuu

SEQ ID NO: 12 - DNA, 31 bp, Artificial Sequence
  TAP_T7_G3C fwd primer
cagtgaattgtaatacgactcactatagggc

SEQ ID NO: 13 - DNA, 87 bp, Artificial Sequence
  IVT_5prime_UTR primer
cagtgaattgtaatacgactcactatagggcgaattaagagagaaaagaagagtaagaagaaatataagacaccggtcgccaccatg

SEQ ID NO: 14 - DNA, 37 bp, Artificial Sequence
  Rev5UTR primer
catggtggcgaccggtgtcttatatttcttcttactc

SEQ ID NO: 15 - DNA, 39 bp, Artificial Sequence
  SphcCas9 ORF fwd primer
caccggtcgccaccatggataagaaatacagcattggac

SEQ ID NO: 16 - DNA, 45 bp, Artificial Sequence
  SphcCas9 ORF rev primer
gccccgcagaaggtctagactatcacaccttcctcttcttcttgg

SEQ ID NO: 17 - DNA, 92 bp, Artificial Sequence
  IVT_3prime_UTR primer
tctagaccttctgcggggcttgccttctggccatgcccttcttctctcccttgcacctgtacctcttggtctttgaataaagcctgagtagg

SEQ ID NO: 18 - DNA, 19 bp, Artificial Sequence
  Fwd3UTR primer
tctagaccttctgcggggc

SEQ ID NO: 19 - DNA, 48 bp, Artificial Sequence
  Rev3UTR2T20
ttttttttttttttttttttcctactcaggctttattcaaagaccaag

SEQ ID NO: 20 - DNA, 39 bp, Artificial Sequence
  GCT7pro_5UTR2
gctaatacgactcactataggttccttaatcgcggatcc

SEQ ID NO: 21 - DNA, 77 bp, Artificial Sequence
  5UTRtemp_T302a-5p
cgactcactataggttccgcgatcgcggatccagcaagtacatccacgtttaagtagatccaccggtcgccaccatg

SEQ ID NO: 22 - DNA, 77 bp, Artificial Sequence
  5UTRtemp_T21-5p
cgactcactataggttccgcgatcgcggatcctcaacatcagtctgataagctaagatcacaccggtcgccaccatg

SEQ ID NO: 23 - DNA, 147 bp, Artificial Sequence
  5UTRtemp_4x302a-5p
cgactcactataggttccgcgatcgcggatccagcaagtacatccacgtttaagtagcaagtacatccacgtttaagtagcaagtacatccacgtttaagtagcaagtacatccacgtttaagtagatcacaccggtcgccaccatg

SEQ ID NO: 24 - DNA, 139 bp, Artificial Sequence
  3UTR120A
ttttttttttttttttttttttttttttttttttttttttttttttttttttttttttttttttttttttttttttttttttttttttttttttttttttttttttttttttttttttttcctactcaggctttattca

SEQ ID NO: 25 - DNA, 64 bp, Artificial Sequence
  T7-sgRNA fwd primer (DMD)
gaaattaatacgactcactataggtatcttacaggaactccgttttagagctagaaatagcaag

SEQ ID NO: 26 - DNA, 67 bp, Artificial Sequence
  T7-sgRNA fwd primer (EGFP)
gaaattaatacgactcactatagggggcacgggcagcttgccgggttttagagctagaaatagcaag

SEQ ID NO: 27 - DNA, 65 bp, Artificial Sequence
  T8-sgRNA fwd primer (EGFP deltaGG)
gaaattaatacgactcactatagggcacgggcagcttgccgggttttagagctagaaatagcaag

SEQ ID NO: 28 - DNA, 67 bp, Artificial Sequence
  T7-sgRNA fwd primer (Alu1)
gaaattaatacgactcactatagggcctgtaatcccagcactttgttttagagctagaaatagcaag

SEQ ID NO: 29 - DNA, 79 bp, Artificial Sequence
  sgRNA+85 rev primer
aaagcaccgactcggtgccactttttcaagttgataacggactagccttattttaacttgctatttctagctctaaaac

SEQ ID NO: 30 - DNA, 19 bp, Artificial Sequence
  T7E1_EGFP Fwd_L
cagccattgccttttatgg

SEQ ID NO: 31 - DNA, 26 bp, Artificial Sequence
  T7E1_EGFP Rev_Univ.2
gtgttctgctggtagtggtcggcgag

SEQ ID NO: 32 - DNA, 25 bp, Artificial Sequence
  T7E1_EGFP Fwd_ORF
tgagcaagggcgaggagctgttcac

SEQ ID NO: 33 - RNA, 4140 nt, Artificial Sequence
  Cas9 coding region
auggauaagaaauacagcauuggacuggacauugggacaaacuccgugggaugggccgugauuacagacgaauacaaagugccuucaaagaaguucaaggugcugggcaacaccgauagacacagcaucaagaaaaaucugauuggagcccugcuguucgacuccggcgagacagcugaagcaacucggcugaaaagaacugcucggagaagguauacccgccgaaagaauaggaucugcuaccugcaggagauuuucagcaacgaaauggccaagguggacgauaguuucuuucaccgccuggaggaaucauuccuggucgaggaagauaagaaacacgagcggcaucccaucuuuggcaacauuguggacgaggucgcuuaucacgaaaaguacccuaccaucuaucaucugaggaagaaacugguggacuccacagauaaagcagaccugcgccugaucuaucuggcccuggcucacaugauuaaguuccggggccauuuucugaucgagggggaucugaacccagacaauucugauguggacaagcuguucauccagcugguccagacauacaaucagcuguuugaggaaaaccccauuaaugcaucuggcguggacgcaaaagccauccugagugccagacugucuaagagucggagacuggagaaccugaucgcucagcugccaggggaaaagaaaaacggccuguuugggaaucugauugcacugucacugggacugacucccaacuucaagagcaauuuugaucuggccgaggacgcuaaacugcagcuguccaaggacaccuaugacgaugaccuggauaaccugcuggcucagaucggggaucaguacgcagaccuguuccuggccgcuaagaaucugucugacgccauccugcugagugauauucugcgcgugaacaccgagauuacaaaagccccccugucagcuagcaugaucaagagauaugacgagcaccaucaggaucugacccugcugaaggcucuggugaggcagcagcugccugagaaguacaaggaaaucuucuuugaucagucuaagaacggauacgccggcuauauugacggcggggcuagucaggaggaguucuacaaguuuaucaaacccauucuggagaagauggauggcacagaggaacugcuggugaaacugaaucgggaagaccugcugaggaagcagcgcacuuuugauaacggaagcaucccucaccagauucaucugggagagcugcacgcaauccugaggcgccaggaagacuucuacccauuucugaaggauaacagggagaagaucgaaaaaauucugacauuccgcauccccuacuaugugggcccucuggcaagaggcaacagccgguuugccuggaugacucgcaaaucugaggaaacaaucacucccuggaacuucgaggaaguggucgauaagggcgcuuccgcacagucuuucauugagcggaugacaaacuucgacaagaaccugccaaacgaaaaagugcugcccaagcacucucugcuguacgaguauuucacagucuauaacgaacugacuaaggugaaauacgucaccgaggggaugagaaagccugccuuccugaguggagaacagaagaaagcuaucguggaccugcuguuuaaaaccaauaggaaggugacagucaagcagcugaaagaggacuauuucaagaaaauugaauguuucgauucuguggagaucaguggcgucgaagacagguuuaacgccucccuggggaccuaccacgaucugcugaagaucauuaaggauaaagacuuccuggacaacgaggaaaaugaggauauccuggaagacauugugcugacccugacacuguuugaggauagggaaaugaucgaggaacgccugaagaccuaugcccaucuguucgaugacaaagugaugaaacagcugaagcgacggagauacacaggauggggccgacugucucggaagcugaucaaugggauucgcgacaaacagaguggaaagaccauccuggacuuucugaaaucagauggcuucgccaaccggaacuucaugcagcugauucacgaugacagccugacauucaaagaggauauccagaaggcacagguguccgggcagggagacucucugcacgagcauaucgcaaaccuggccggcagcccugccaucaagaaagggauucugcagaccgugaaggugguggacgagcuggugaaagucaugggaagacauaagccagaaaacaucgugauugagauggccagggaaaaucagaccacacagaaaggccagaagaacucaagggagcgcaugaaaagaaucgaggaaggaauuaaggaacugggcagccagauccugaaagagcaccccguggaaaacacacagcugcagaaugagaagcuguaucuguacuaucugcagaauggacgcgauauguacguggaccaggagcuggauauuaaccgacuguccgauuacgacguggaucauaucgucccacagucauuccugaaagaugacagcauugacaauaaggugcugacccgcucugacaaaaaccgaggcaagagugauaauguccccucagaggaaguggucaagaaaaugaagaacuacuggaggcagcugcugaaugccaaacugaucacacagcgaaaguuugauaaccugacuaaagcugagcggggaggccugagugaacuggacaaagcaggcuucauuaagcgacagcugguggagacacggcagaucacaaagcacgucgcccagauucuggauucaagaaugaacacuaaguacgaugagaaugacaaacugaucagagaagugaaggucauuacccugaagucaaaacuggugagcgacuuucggaaagauuuccaguuuuauaaggucagagagaucaacaacuaccaccaugcucaugacgcauaccugaacgcaguggucggcacagcccugauuaagaaauacccuaaacuggaguccgaguucguguacggggacuauaagguguacgaugucagaaaaaugaucgccaagucugagcaggaaauuggcaaagccacugcuaaguauuucuuuuacaguaacaucaugaauuucuuuaagacugagaucacccuggcaaauggggaaauccgaaagcggccacugauugagacuaacggcgagacaggagaaaucgugugggacaaaggaagagauuuugcuaccgugaggaagguccugagcaugccccaagugaauauugucaagaaaacagaggugcagacugggggauucaguaaggaaucaauucugccuaaacgcaacuccgauaagcugaucgcccgaaagaaagacugggaccccaagaaguauggcggguucgacuccccaacuguggcuuacucuguccugguggucgcaaagguggagaagggaaaaagcaagaaacugaaauccgucaaggaacugcugggcaucaccauuauggagcgcagcuccuucgaaaagaauccuaucgauuuucuggaggccaaaggcuauaaggaagugaagaaagaccugaucaucaagcugccaaaguacucacuguuugagcuggaaaacgggagaaagaggaugcuggcaagcgccggggagcugcagaaaggaaaugaacuggcccugcccuccaaguacgugaacuuccuguaucuggcuagccacuacgagaagcugaaaggguccccugaggauaacgaacagaaacagcuguuuguggagcagcacaagcauuaucuggacgagaucauugaacagauuagcgaguucuccaaaagagugauccuggcugacgcaaaucuggauaagguccugagcgcauacaacaaacaccgggauaagccaaucagagagcaggccgaaaauaucauucaucuguucacucugaccaaccugggagcccccgcagccuucaaguauuuugacacuaccaucgaucgcaaacgauacacaagcacuaaggaggugcuggacgcuacccugauucaucagagcauuacuggccuguaugaaacaaggauugaccugucucagcugggcggcgacuccggagcugaccccaagaagaagaggaagguguga

SEQ ID NO: 34 - RNA, 215 nt, Artificial Sequence
  3′UTR
uagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 57 nt, Artificial Sequence
  5′UTR_Control Cas9 mRNA
gggcgaauuaagagagaaaagaagaguaagaagaaauauaagacaccggucgccacc

SEQ ID NO: 36 - RNA, 4476 nt, Artificial Sequence
  Kt-Cas9 mRNA
ggauccgugaucggaaacgugagauccaccucagauccgcuaggacacccgcagaucgagaagaaggcgaauuaagagagaaaagaagaguaagaagaaauauaagacaccggucgccaccauggauaagaaauacagcauuggacuggacauugggacaaacuccgugggaugggccgugauuacagacgaauacaaagugccuucaaagaaguucaaggugcugggcaacaccgauagacacagcaucaagaaaaaucugauuggagcccugcuguucgacuccggcgagacagcugaagcaacucggcugaaaagaacugcucggagaagguauacccgccgaaagaauaggaucugcuaccugcaggagauuuucagcaacgaaauggccaagguggacgauaguuucuuucaccgccuggaggaaucauuccuggucgaggaagauaagaaacacgagcggcaucccaucuuuggcaacauuguggacgaggucgcuuaucacgaaaaguacccuaccaucuaucaucugaggaagaaacugguggacuccacagauaaagcagaccugcgccugaucuaucuggcccuggcucacaugauuaaguuccggggccauuuucugaucgagggggaucugaacccagacaauucugauguggacaagcuguucauccagcugguccagacauacaaucagcuguuugaggaaaaccccauuaaugcaucuggcguggacgcaaaagccauccugagugccagacugucuaagagucggagacuggagaaccugaucgcucagcugccaggggaaaagaaaaacggccuguuugggaaucugauugcacugucacugggacugacucccaacuucaagagcaauuuugaucuggccgaggacgcuaaacugcagcuguccaaggacaccuaugacgaugaccuggauaaccugcuggcucagaucggggaucaguacgcagaccuguuccuggccgcuaagaaucugucugacgccauccugcugagugauauucugcgcgugaacaccgagauuacaaaagccccccugucagcuagcaugaucaagagauaugacgagcaccaucaggaucugacccugcugaaggcucuggugaggcagcagcugccugagaaguacaaggaaaucuucuuugaucagucuaagaacggauacgccggcuauauugacggcggggcuagucaggaggaguucuacaaguuuaucaaacccauucuggagaagauggauggcacagaggaacugcuggugaaacugaaucgggaagaccugcugaggaagcagcgcacuuuugauaacggaagcaucccucaccagauucaucugggagagcugcacgcaauccugaggcgccaggaagacuucuacccauuucugaaggauaacagggagaagaucgaaaaaauucugacauuccgcauccccuacuaugugggcccucuggcaagaggcaacagccgguuugccuggaugacucgcaaaucugaggaaacaaucacucccuggaacuucgaggaaguggucgauaagggcgcuuccgcacagucuuucauugagcggaugacaaacuucgacaagaaccugccaaacgaaaaagugcugcccaagcacucucugcuguacgaguauuucacagucuauaacgaacugacuaaggugaaauacgucaccgaggggaugagaaagccugccuuccugaguggagaacagaagaaagcuaucguggaccugcuguuuaaaaccaauaggaaggugacagucaagcagcugaaagaggacuauuucaagaaaauugaauguuucgauucuguggagaucaguggcgucgaagacagguuuaacgccucccuggggaccuaccacgaucugcugaagaucauuaaggauaaagacuuccuggacaacgaggaaaaugaggauauccuggaagacauugugcugacccugacacuguuugaggauagggaaaugaucgaggaacgccugaagaccuaugcccaucuguucgaugacaaagugaugaaacagcugaagcgacggagauacacaggauggggccgacugucucggaagcugaucaaugggauucgcgacaaacagaguggaaagaccauccuggacuuucugaaaucagauggcuucgccaaccggaacuucaugcagcugauucacgaugacagccugacauucaaagaggauauccagaaggcacagguguccgggcagggagacucucugcacgagcauaucgcaaaccuggccggcagcccugccaucaagaaagggauucugcagaccgugaaggugguggacgagcuggugaaagucaugggaagacauaagccagaaaacaucgugauugagauggccagggaaaaucagaccacacagaaaggccagaagaacucaagggagcgcaugaaaagaaucgaggaaggaauuaaggaacugggcagccagauccugaaagagcaccccguggaaaacacacagcugcagaaugagaagcuguaucuguacuaucugcagaauggacgcgauauguacguggaccaggagcuggauauuaaccgacuguccgauuacgacguggaucauaucgucccacagucauuccugaaagaugacagcauugacaauaaggugcugacccgcucugacaaaaaccgaggcaagagugauaauguccccucagaggaaguggucaagaaaaugaagaacuacuggaggcagcugcugaaugccaaacugaucacacagcgaaaguuugauaaccugacuaaagcugagcggggaggccugagugaacuggacaaagcaggcuucauuaagcgacagcugguggagacacggcagaucacaaagcacgucgcccagauucuggauucaagaaugaacacuaaguacgaugagaaugacaaacugaucagagaagugaaggucauuacccugaagucaaaacuggugagcgacuuucggaaagauuuccaguuuuauaaggucagagagaucaacaacuaccaccaugcucaugacgcauaccugaacgcaguggucggcacagcccugauuaagaaauacccuaaacuggaguccgaguucguguacggggacuauaagguguacgaugucagaaaaaugaucgccaagucugagcaggaaauuggcaaagccacugcuaaguauuucuuuuacaguaacaucaugaauuucuuuaagacugagaucacccuggcaaauggggaaauccgaaagcggccacugauugagacuaacggcgagacaggagaaaucgugugggacaaaggaagagauuuugcuaccgugaggaagguccugagcaugccccaagugaauauugucaagaaaacagaggugcagacugggggauucaguaaggaaucaauucugccuaaacgcaacuccgauaagcugaucgcccgaaagaaagacugggaccccaagaaguauggcggguucgacuccccaacuguggcuuacucuguccugguggucgcaaagguggagaagggaaaaagcaagaaacugaaauccgucaaggaacugcugggcaucaccauuauggagcgcagcuccuucgaaaagaauccuaucgauuuucuggaggccaaaggcuauaaggaagugaagaaagaccugaucaucaagcugccaaaguacucacuguuugagcuggaaaacgggagaaagaggaugcuggcaagcgccggggagcugcagaaaggaaaugaacuggcccugcccuccaaguacgugaacuuccuguaucuggcuagccacuacgagaagcugaaaggguccccugaggauaacgaacagaaacagcuguuuguggagcagcacaagcauuaucuggacgagaucauugaacagauuagcgaguucuccaaaagagugauccuggcugacgcaaaucuggauaagguccugagcgcauacaacaaacaccgggauaagccaaucagagagcaggccgaaaauaucauucaucuguucacucugaccaaccugggagcccccgcagccuucaaguauuuugacacuaccaucgaucgcaaacgauacacaagcacuaaggaggugcuggacgcuacccugauucaucagagcauuacuggccuguaugaaacaaggauugaccugucucagcugggcggcgacuccggagcugaccccaagaagaagaggaaggugugauagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 37 - RNA, 653 nt, Artificial Sequence
  L7Ae mRNA
gggcgaauuaagagagaaaagaagaguaagaagaaauauaagacaccggucgccaccauguacgugagauuugagguuccugaggacaugcagaacgaagcucugagucugcuggagaagguuagggagagcgguaagguaaagaaagguaccaacgagacgacaaaggcuguggagaggggacuggcaaagcucguuuacaucgcagaggauguugacccgccugagaucguugcucaucugccccuccucugcgaggagaagaaugugccguacauuuacguuaaaagcaagaacgaccuuggaagggcugugggcauugaggugccaugcgcuucggcagcgauaaucaacgagggagagcugagaaaggagcuuggaagccuuguggagaagauuaaaggccuucagaagagaucucauaugcaucucgagugauagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - RNA, 658 nt, Artificial Sequence
  miR-21-responsive L7Ae
gguuccuuaaucgcggauccucaacaucagucugauaagcuaagaucacaccggucgccaccauguacgugagauuugagguuccugaggacaugcagaacgaagcucugagucugcuggagaagguuagggagagcgguaagguaaagaaagguaccaacgagacgacaaaggcuguggagaggggacuggcaaagcucguuuacaucgcagaggauguugacccgccugagaucguugcucaucugccccuccucugcgaggagaagaaugugccguacauuuacguuaaaagcaagaacgaccuuggaagggcugugggcauugaggugccaugcgcuucggcagcgauaaucaacgagggagagcugagaaaggagcuuggaagccuuguggagaagauuaaaggccuucagaagagaucucauaugcaucucgagugauagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 39 - RNA, 992 nt, Artificial Sequence
  Tag BFP mRNA
gggcgaauuaagagagaaaagaagaguaagaagaaauauaagacaccggucgccaccaugggauccagcgagcugauuaaggagaacaugcacaugaagcuguacauggagggcaccguggacaaccaucacuucaagugcacauccgagggcgaaggcaagcccuacgagggcacccagaccaugagaaucaagguggucgagggcggcccucuccccuucgccuucgacauccuggcuacuagcuuccucuacggcagcaagaccuucaucaaccacacccagggcauccccgacuucuucaagcaguccuucccugagggcuucacaugggagagagucaccacauacgaagacgggggcgugcugaccgcuacccaggacaccagccuccaggacggcugccucaucuacaacgucaagaucagaggggugaacuucacauccaacggcccugugaugcagaagaaaacacucggcugggaggccuucaccgagacgcuguaccccgcugacggcggccuggaaggcagaaacgacauggcccugaagcucgugggcgggagccaucugaucgcaaacaucaagaccacauauagauccaagaaacccgcuaagaaccucaagaugccuggcgucuacuauguggacuacagacuggaaagaaucaaggaggccaacaacgagaccuacgucgagcagcacgagguggcaguggccagauacugcgaccucccuagcaaacuggggcacagaucucauaugcaucucgagugauagucuagaccuucugcggggcuugccuucuggccaugcccuucuucucucccuugcaccuguaccucuuggucuuugaauaaagccugaguaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - RNA, 12 nt, Artificial Sequence
  boxC motif
ggcgugaugagc

SEQ ID NO: 41 - RNA, 34 nt, Artificial Sequence
  kink-loop
agauccgggugugaacggugaucacccgagaucc

SEQ ID NO: 42 - RNA, 45 nt, Artificial Sequence
  kink-loop2
agauccggacguacgugugaacggugaucacguacgccgagaucc

SEQ ID NO: 43 - RNA, 37 nt, Artificial Sequence
  Threonyl-tRNA synthetase binding sequence
ggcguaugugaucuuucgugugggucaccacugcgcc

SEQ ID NO: 44 - RNA, 50 nt, Artificial Sequence
  R9-2
gggugcuucgagcguaggaagaaagccgggggcugcagauaauguauagc